(19) United States
(12) Patent Application Publication
(10) Pub. No.: US 2020/0165342 A1
(43) Pub. Date: May 28, 2020

(54) HLA-DR CAR-T COMPOSITIONS AND METHODS OF MAKING AND USING THE SAME

(71) Applicant: Eutilex Co., Ltd., Seoul (KR)

(72) Inventors: Byoung S. Kwon, Seoul (KR); Chungyong Han, Seoul (KR)

(21) Appl. No.: 16/485,598

(22) PCT Filed: Feb. 21, 2018
(86) PCT No.: PCT/IB2018/000227

§ 371 Date: Aug. 13, 2019

Related U.S. Application Data
(60) Provisional application No. 62/461,632, filed on Feb. 21, 2017.

(51) Int. Cl. C07K 16/28; C12N 5/0783; A61K 35/17; A61P 35/00

(52) U.S. Cl. CPC C07K 16/2833; C12N 5/0638; A61K 35/17; A61P 35/00; C07K 2317/24; C07K 2317/92; C07K 2319/33; C07K 2317/622; C07K 2319/03; C07K 2319/02; C07K 2319/30

(57) ABSTRACT

Provided are CAR-T compositions that are directed to HLA-DR. Certain provided HLA-DR CAR compositions exhibit low affinity for a polymorphic region of HLA-DR of a subject. Various in vitro and in vivo methods and reagents related to HLA-DR CAR-T are also provided. Methods described herein can include, for example, characterization of HLA-DR binding, proliferation of T-cells, as well as prevention and/or therapeutic treatment of cancer using a HLA-DR CAR-T composition provided herein.

### CROSS-REFERENCE TO RELATED APPLICATIONS

This application is a National Stage application under 35 U.S.C. § 371 of International Patent Application No. PCT/M2018/000227, filed on Feb. 21, 2018, which claims priority to and the benefit of U.S. Patent Application No. 62/461,632, filed on Feb. 21, 2017, the disclosure of which are incorporated herein by reference in their entirety.

### STATEMENT REGARDING SEQUENCE LISTING

The Sequence Listing associated with this application is provided in text form in lieu of a paper copy, and is hereby incorporated by reference into the specification. The name of the text file containing the Sequence Listing is sequencelisting.txt. The text file is 14.1 KB, and was created and submitted electronically via EFS-Web on Nov. 4, 2019.

### BACKGROUND

Cancer remains one of the leading causes of death in the world. Recent statistics report that 13% of the world population dies from cancer. According to estimates from the International Agency for Research on Cancer (IARC), in 2012 there were 14.1 million new cancer cases and 8.2 million cancer deaths worldwide. By 2030, the global burden is expected to grow to 21.7 million new cancer cases and 13 million cancer deaths due to population growth and aging and exposure to risk factors such as smoking, unhealthy diet and physical inactivity. Further, pain and medical expenses for cancer treatment cause reduced quality of life for both cancer patients and their families.

T cells engineered with chimeric antigen receptors (CAR-T) have great therapeutic potential for treating diseases such as cancers. CAR-T therapeutics confer powerful target affinity and signaling function on T cell. However, the impressive efficacy of CAR-T therapies are frequently accompanied by severe side effects, such as cytokine release syndrome (CRS). Thus there remains an unmet need to develop CAR-T therapeutics and strategies that have reduced side effects.

### SUMMARY

The present disclosure provides, among other things, engineered T cells that express a chimeric antigen receptor (CAR) that includes a HLA-DR antigen binding domain. The present disclosure provides the insight that a CAR that includes an HLA-DR antigen binding domain (HLA-DR CAR) can be selected, engineered and/or optimized based on the binding characteristics of the HLA-DR binding domain to a T cell from a subject. In some embodiments, a HLA-DR binding domain is specific for a polymorphic epitope of HLA-DR. The present disclosure encompasses a recognition that a HLA-DR CAR that binds to a cell (e.g., a T cell) from a subject with low affinity can provide effective therapy for treating certain diseases and/or disorders.

In some embodiments, the present disclosure provides a T cell comprising a chimeric antigen receptor (CAR), wherein the CAR comprises a HLA-DR antigen binding domain, wherein the T cell is autologous to a subject, and wherein the HLA-DR antigen binding domain binds to a T cell from the subject with low affinity. In some embodiments, a HLA-DR antigen binding domain is a MVR-scFv or a variant thereof.

In some embodiments, the present disclosure provides methods of treating cancer that include administering to a subject a composition that comprises or delivers a HLA-DR CAR cell of the present disclosure.

In some embodiments, the present disclosure provides methods of producing an autologous engineered T cell, comprising: (a) obtaining a HLA-DR antigen binding domain, wherein the HLA-DR antigen binding domain binds to HLA-DR from a subject with low affinity, and (b) expressing a chimeric antigen receptor (CAR) comprising the HLA-DR antigen binding domain in a T cell obtained from the subject, thereby producing the autologous engineered T cell.

In some embodiments, the present disclosure provides methods of preparing an autologous engineered T cell, comprising: providing or obtaining an analysis of binding of a HLA-DR antigen binding domain to a T cell from a subject; and if the binding is less than a threshold value, engineering a T cell from the subject to express a CAR comprising the HLA-DR antigen binding domain. In some embodiments, an autologous engineered T cell, expands during 12 days of culture with appropriate stimulation at least 15-fold, at least 20-fold, at least 25-fold, or more. In some embodiments, an autologous engineered T cell that comprises a CAR comprising a HLA-DR antigen binding domain that binds to a T cell from a subject with a binding that is less than a threshold value expands during 12 days of culture with appropriate stimulation at least 15-fold, at least 20-fold, at least 25-fold, or more. In some embodiments, an autologous engineered T cell that comprises a CAR comprising a HLA-DR antigen binding domain that binds to a T cell from a subject with a binding that is less than a threshold value expands during 12 days of culture with appropriate stimulation at least 20-fold. In some embodiments, an appropriate stimulation includes exposing the T cell to a CD3-specific antibody and/or a HLA-DR-expressing cell.

In some embodiments, an analysis of binding of a HLA-DR antigen binding domain to a T cell from a subject is a direct measurement of binding affinity (e.g., K_{D}). In some embodiments, an analysis of binding of a HLA-DR antigen binding domain to a T cell from a subject is a measure of functional avidity of a HLA-DR antigen binding domain to a T cell. In some embodiments, the functional avidity inversely correlates with the antigen dose that is needed to trigger a T-cell response. In some embodiments, a measure of functional avidity of a HLA-DR antigen binding domain to a T cell includes ex vivo quantification of T cell functions such as, for example, IFN-γ production, cytotoxic activity (ability to lyse target cells), or proliferation. In some embodiments, a measure of functional avidity of a HLA-DR antigen binding domain to a T cell includes determining a concentration of a HLA-DR antigen binding domain needed to induce a half-maximum response (EC₅₀) of T cells.

In some embodiments, provided methods include preparation and/or production of an autologous engineered T cell that expresses an HLA-DR CAR that includes a HLA-DR antigen binding domain.

In some embodiments, a HLA-DR antigen binding domain comprises a heavy chain variable region having one, two or three heavy chain CDRs comprising a heavy chain CDR sequence as set forth in any one of SEQ ID NOs: 2-4; and a light chain variable region having one, two or three light chain CDRs comprising a light chain CDR sequence as set forth in any one of SEQ ID NOs: 6-8.

In some embodiments, a HLA-DR antigen binding domain comprises a heavy chain variable region having a heavy chain CDR1 as set forth in SEQ ID NO:2; a heavy chain CDR2 as set forth in SEQ ID NO:3; and a heavy chain CDR3 as set forth in SEQ ID NO:4; and a light chain variable region having a light chain CDR1 as set forth in SEQ ID NO: 6; a light chain CDR2 as set forth in SEQ ID NO:7; and a light chain CDR3 as set forth in SEQ ID NO:8.

In some embodiments, a HLA-DR antigen binding domain comprises a heavy chain variable region with an amino acid sequence that is 80%, 85%, 90%, 91%, 92%, 93%, 94%, 95%, 96%, 97%, 98%, 99% or 100% identical to a sequence as set forth in SEQ ID NO: 1 and a light chain variable region with an amino acid sequence that is 80%, 85%, 90%, 91%, 92%, 93%, 94%, 95%, 96%, 97%, 98%, 99%, or 100% identical to a sequence as set forth in SEQ ID NO: 5

In some embodiments, a HLA-DR CAR comprises i) a HLA-DR antigen binding domain comprises a heavy chain variable region having a heavy chain CDR1 as set forth in SEQ ID NO:2; a heavy chain CDR2 as set forth in SEQ ID NO:3; and a heavy chain CDR3 as set forth in SEQ ID NO:4; and a light chain variable region having a light chain CDR1 as set forth in SEQ ID NO: 6; a light chain CDR2 as set forth in SEQ ID NO:7; and a light chain CDR3 as set forth in SEQ ID NO:8; ii) a transmembrane domain; and iii) an intracellular signaling domain, which leads to T cell activation when an antigen binds to the HLA-DR antigen binding domain.

In some embodiments, a HLA-DR CAR comprises i) a HLA-DR antigen binding domain comprises a heavy chain variable region with an amino acid sequence that is 80%, 85%, 90%, 91%, 92%, 93%, 94%, 95%, 96%, 97%, 98%, 99% or 100% identical to a sequence as set forth in SEQ ID NO: 1 and a light chain variable region with an amino acid sequence that is 80%, 85%, 90%, 91%, 92%, 93%, 94%, 95%, 96%, 97%, 98%, 99%, or 100% identical to a sequence as set forth in SEQ ID NO: 5; ii) a transmembrane domain; and iii) an intracellular signaling domain, which leads to T cell activation when an antigen binds to the HLA-DR antigen binding domain.

In some embodiments, a HLA-DR CAR further comprises an intracellular domain of a T cell receptor-ζ (TCR-ζ). In some embodiments, the T cell receptor-ζ (TCR-ζ) is or comprises a CD3 domain (e.g., CD3zeta domain). In some embodiments, a HLA-DR CAR further comprises a CD8α transmembrane domain and/or a 4-1BB signaling domain.

In some embodiments, a HLA-DR CAR comprises a sequence that is at least 70%, 75%, 80%, 85%, 90%, 95%, or 99% identical to a sequence as set forth in SEQ ID NO: 9. In some embodiments, a HLA-DR CAR comprises or consists of a sequence as set forth in SEQ ID NO: 9.

In some embodiments, a T cell comprising HLA-DR CAR of the present disclosure has a killing efficiency for a B cell that is two times or three times lower than a killing efficiency of the T cell for an EBV LCL.

In some embodiments, the present disclosure provides a pharmaceutical composition comprising a HLA-DR CAR T cell of the present disclosure and a pharmaceutically acceptable carrier.

In some embodiments, the present disclosure provides methods of producing an autologous engineered T cell, comprising: (a) obtaining a HLA-DR antigen binding domain, wherein HLA-DR antigen binding domain binds to HLA-DR from a subject with low affinity, and (b) expressing a chimeric antigen receptor (CAR) comprising the HLA-DR antigen binding domain in a T cell obtained from the subject, thereby producing the autologous engineered T cell and further comprising culturing the autologous engineered T cell in vitro for at least 8 days, 9 days, 10 days, 11 days, or 12 days.

In some embodiments, the present disclosure provides methods of preparing an autologous engineered T cell, comprising: providing or obtaining an analysis of binding of a HLA-DR antigen binding domain to a T cell from a subject; and if the binding is less than a threshold value, engineering a T cell from the subject to express a CAR comprising the HLA-DR antigen binding domain and further comprising culturing the autologous engineered T cell in vitro for at least 8 days, 9 days, 10 days, 11 days, or 12 days. In some embodiments, an autologous engineered T cell, expands during 12 days of culture with appropriate stimulation at least 15-fold, at least 20-fold, at least 25-fold, or more. In some embodiments, an autologous engineered T cell that comprises a CAR comprising a HLA-DR antigen binding domain that binds to a T cell from a subject with a binding that is less than a threshold value expands during 12 days of culture with appropriate stimulation at least 15-fold, at least 20-fold, at least 25-fold, or more. In some embodiments, an autologous engineered T cell that comprises a CAR comprising a HLA-DR antigen binding domain that binds to a T cell from a subject with a binding that is less than a threshold value expands during 12 days of culture with appropriate stimulation at least 20-fold. In some embodiments, an appropriate stimulation is includes exposing the T cell to a CD3-specific antibody and/or a HLA-DR-expressing cell.

In some embodiments, the step of culturing in the provided methods produces a population of autologous engineered T cells with reduced surface expression of the CAR relative to a population of the autologous engineered T cells that has been cultured in vitro for 2 days.

In some embodiments, the step of culturing in the provided methods produces a population of autologous engineered T cells with reduced toxicity towards normal B cells relative to a population of the autologous engineered T cells that has been cultured in vitro for 2 days.

In some embodiments, the step of culturing in the provided methods produces a population of autologous engineered T cells that has enhanced selectivity for malignant cells over to non-malignant cells relative to a population of the autologous engineered T cells that has been cultured in vitro for 2 days.

In some embodiments, an autologous engineered T cell in the context of the present disclosure exhibits granual transfer to EBV LCLs that is at least two times more than the granual transfer of the engineered T cell to normal B cells from the subject.

In some embodiments, the present disclosure provides methods of treating and/or preventing cancer comprising administering to a subject in need thereof a composition that comprises or delivers the autologous engineered T cell prepared by any of the methods provided herein. In some embodiments, a cancer cell expresses HLA-DR antigen. In some embodiments, a cancer cell has increased expression of HLA-DR antigen relative to a non-cancer cell from a subject. In some embodiments, a cancer cell has at least 1.5-fold, 2-fold, 3-fold, 4-fold, 5-fold, or 6-fold higher expression of HLA-DR antigen relative to a non-cancer cell from a subject. In some certain embodiments, a cancer suitable for treatment with compositions and methods of the present disclosure has an at least 2-fold higher expression of HLA-DR antigen relative to a normal cell of the same type from a subject.

In some embodiments, the provided methods can be used to treat or prevent a cancer selected from selected from a bladder cancer, breast cancer, cervical cancer, colon cancer, endometrial cancer, esophageal cancer, fallopian tube cancer, gall bladder cancer, gastrointestinal cancer, head and neck cancer, hematological cancer, laryngeal cancer, liver cancer, lung cancer, lymphoma, melanoma, mesothelioma, ovarian cancer, primary peritoneal cancer, salivary gland cancer, sarcoma, stomach cancer, thyroid cancer, pancreatic cancer, and prostate cancer.

In some embodiments, the present disclosure provides methods of treating and/or preventing a hematologic cancer. In some embodiments, a hematologic cancer is selected from B-cell acute lymphoid leukemia (“BALL”), T-cell acute lymphoid leukemia (“TALL”), acute lymphoid leukemia (ALL), chronic myelogenous leukemia (CML), chronic lymphocytic leukemia (CLL), B cell prolymphocytic leukemia, blastic plasmacytoid dendritic cell neoplasm, Burkitt's lymphoma, diffuse large B cell lymphoma, follicular lymphoma, hairy cell leukemia, small cell-follicular lymphoma, large cell-follicular lymphoma, a malignant lymphoproliferative condition, MALT lymphoma, mantle cell lymphoma, Marginal zone lymphoma, multiple myeloma, myelodysplasia, non-Hodgkin's lymphoma, plasmablastic lymphoma, plasmacytoid dendritic cell neoplasm, and Waldenstrom macroglobulinemia.

In some embodiments, a provided method of treatment of the present disclosure will include those where a subject has been administered, or will be administered, one or more additional anticancer therapies selected from ionizing radiation, a chemotherapeutic agent, an antibody agent, and a cell-based therapy, such that the subject receives treatment with both.

In some embodiments, the present disclosure provides T cells comprising nucleic acid molecules encoding a HLA-DR CAR. In some embodiments, the present disclosure provides T cells comprising vectors that include a nucleic acid molecule encoding a HLA-DR CAR.

In some embodiments, the present disclosure provides pharmaceutical compositions that include a T cell comprising a HLA-DR CAR and a pharmaceutically acceptable carrier. In some embodiments a T cell comprising a HLA-DR CAR is an autologous cell. In some embodiments, a HLA-DR binding domain of an HLA-DR CAR has low affinity for a T cell from a subject that is to be administered a pharmaceutical composition. In some embodiments, the present disclosure provides pharmaceutical compositions that include a T cell comprising a nucleic acid and/or vector encoding a HLA-DR CAR and a pharmaceutically acceptable carrier. In some embodiments a T cell comprising a nucleic acid and/or vector encoding a HLA-DR CAR is an autologous T cell. In some embodiments, a HLA-DR binding domain of an HLA-DR CAR has low affinity for a T cell from a subject to be administered a pharmaceutical composition.

In some embodiments, the present disclosure provides methods of producing a therapeutic preparation, comprising: providing or obtaining an analysis of avidity of an engineered T cell comprising a CAR comprising a HLA-DR antigen binding domain for an HLA-DR antigen of a subject, and if the avidity is less than a threshold value, producing a therapeutic preparation comprising the engineered T cell. In some embodiments, an analysis of avidity of an engineered T cell comprising a CAR comprising a HLA-DR antigen binding domain for an HLA-DR antigen of a subject is an analysis of functional avidity. In some embodiments, a measure of functional avidity of a HLA-DR antigen binding domain to a T cell includes ex vivo quantification of T cell functions such as, for example, IFN-γ production, cytotoxic activity (ability to lyse target cells), or proliferation.

In some embodiments, a method for producing a therapeutic preparation comprises: providing or obtaining an analysis of functional avidity of an engineered T cell comprising a CAR comprising a HLA-DR antigen binding domain for an HLA-DR antigen of a subject, and if the functional avidity is less than a threshold value, producing a therapeutic preparation comprising the engineered T cell. In some embodiments, a measure of functional avidity is proliferation of an engineered T cell when cultured for at least 8 days, 10 days, 12 days or 14 days with an appropriate stimulation. In some embodiments, an appropriate stimulation includes exposing the T cell to a CD3-specific antibody and/or a HLA-DR-expressing cell. In some embodiments, a threshold value of functional avidity is at least 15-fold, 20-fold, 25-fold proliferation.

In some embodiments, a method for producing a therapeutic preparation comprises: providing or obtaining an analysis of functional avidity of an engineered T cell comprising a CAR comprising a HLA-DR antigen binding domain for an HLA-DR antigen of a subject, and if the functional avidity is less than a threshold value, producing a therapeutic preparation comprising the engineered T cell, wherein the threshold value is at least 15-fold, 20-fold, 25-fold proliferation of an engineered T cell when cultured for at least 12 days with a CD3-specific antibody and/or a HLA-DR-expressing cell.

In some embodiments, the present disclosure provides methods of treating a subject in need thereof, the method comprising administering to the subject a composition that comprises or delivers a T cell comprising a HLA-DR CAR. In some embodiments a T cell comprising a HLA-DR CAR is an autologous T cell. In some embodiments, a HLA-DR binding domain of an HLA-DR CAR has low affinity for a T cell from a subject that is to be administered a pharmaceutical composition. In some embodiments, the present disclosure provides methods of treating a subject in need thereof, the method comprising administering to the subject a composition that comprises or delivers a T cell comprising a nucleic acid and/or vector encoding a HLA-DR CAR. In some embodiments a T cell comprising a nucleic acid and/or vector encoding a HLA-DR CAR is an autologous T cell. In some embodiments, a HLA-DR binding domain of an HLA-DR CAR has low affinity for a T cell from a subject to be administered a pharmaceutical composition. In some embodiments, a subject has or is at risk for developing cancer.

In some embodiments, the present disclosure provides methods of inducing an immune response in a subject in need thereof, the method comprising administering to the subject a composition that comprises or delivers a T cell comprising a HLA-DR CAR. In some embodiments a T cell comprising a HLA-DR CAR is an autologous T cell. In some embodiments, a HLA-DR binding domain of an HLA-DR CAR has low affinity for a T cell from a subject that is to be administered a pharmaceutical composition. In some embodiments, the present disclosure provides methods of inducing an immune response in a subject in need thereof, the method comprising administering to the subject a composition that comprises or delivers a T cell comprising a nucleic acid and/or vector encoding a HLA-DR CAR. In some embodiments a T cell comprising a nucleic acid and/or vector encoding a HLA-DR CAR is an autologous T cell. In some embodiments, a HLA-DR binding domain of an HLA-DR CAR has low affinity for a T cell from a subject to be administered a pharmaceutical composition. In some embodiments, a subject has or is at risk for developing cancer.

In some embodiments, the present disclosure provides methods of enhancing an immune response in a subject in need thereof, the method comprising administering to the subject a composition that comprises or delivers a T cell comprising a HLA-DR CAR. In some embodiments a T cell comprising a HLA-DR CAR is an autologous T cell. In some embodiments, a HLA-DR binding domain of an HLA-DR CAR has low affinity for a T cell from a subject that is to be administered a pharmaceutical composition. In some embodiments, the present disclosure provides methods of enhancing an immune response in a subject in need thereof, the method comprising administering to the subject a composition that comprises or delivers a T cell comprising a nucleic acid and/or vector encoding a HLA-DR CAR. In some embodiments a T cell comprising a nucleic acid and/or vector encoding a HLA-DR CAR is an autologous T cell. In some embodiments, a HLA-DR binding domain of an HLA-DR CAR has low affinity for a T cell from a subject to be administered a pharmaceutical composition. In some embodiments, a subject has or is at risk for developing cancer.

In some embodiments, cancers suitable for treatment in the present disclosure can include, for example, hematologic cancers. In some embodiments, a hematologic cancer is leukemia. In some embodiments, a cancer is selected from the group consisting of one or more of B-cell acute lymphoid leukemia (“BALL”), T-cell acute lymphoid leukemia (“TALL”), acute lymphoid leukemia (ALL), chronic myelogenous leukemia (CML), chronic lymphocytic leukemia (CLL), B cell prolymphocytic leukemia, blastic plasmacytoid dendritic cell neoplasm, Burkitt's lymphoma, diffuse large B cell lymphoma, follicular lymphoma, hairy cell leukemia, small cell-follicular lymphoma, large cell-follicular lymphoma, a malignant lymphoproliferative condition, MALT lymphoma, mantle cell lymphoma, Marginal zone lymphoma, multiple myeloma, myelodysplasia, non-Hodgkin's lymphoma, plasmablastic lymphoma, plasmacytoid dendritic cell neoplasm, and Waldenstrom macroglobulinemia.

In some embodiments, the present disclosure provides methods that include administering to the subject a composition that comprises or delivers a T cell comprising a HLA-DR CAR to a subject that has been administered, or will be administered, one or more additional anticancer therapies. In some embodiments, the present disclosure provides methods that include administering to the subject a composition that comprises or delivers a T cell comprising a HLA-DR CAR to a subject that has been administered or will be administered one or more of ionizing radiation, a chemotherapeutic agent, an antibody agent, and a cell-based therapy, such that the subject receives treatment with both.

In some embodiments, the present disclosure provides methods that include administering to the subject a composition that comprises or delivers a T cell comprising a nucleic acid encoding a HLA-DR CAR to a subject that has been administered, or will be administered, one or more additional anticancer therapies. In some embodiments, the present disclosure provides methods that include administering to the subject a composition that comprises or delivers a T cell comprising a nucleic acid encoding a HLA-DR CAR to a subject that has been administered or will be administered one or more of ionizing radiation, a chemotherapeutic agent, an antibody agent, and a cell-based therapy, such that the subject receives treatment with both.

In some embodiments, the present disclosure provides methods for treating or preventing cancer in a subject in need thereof that includes administering to the subject a composition that includes a therapeutically effective amount of T cells comprising a HLA-DR CAR produced by any of the method described herein. In some embodiments, a composition includes at least 10⁶, at least 10⁷, at least 10⁸, at least 10⁹, at least 10¹⁰ cells, or more than 10¹⁰ T cells comprising a HLA-DR CAR. In some embodiments, T cells comprising a HLA-DR CAR are CD4⁺ T cells and/or CD8⁺ T cells.

In some embodiments, the present disclosure provides methods for treating or preventing cancer in a subject in need thereof that includes administering to the subject a composition that includes a therapeutically effective amount of T cells comprising a nucleic acid encoding a HLA-DR CAR produced by any of the method described herein. In some embodiments, a composition includes at least 10⁶, at least 10⁷, at least 10⁸, at least 10⁹, at least 10¹⁰ cells, or more than 10¹⁰ T cells comprising a nucleic acid encoding HLA-DR CAR. In some embodiments, T cells comprising a nucleic acid encoding HLA-DR CAR are CD4⁺ T cells and/or CD8⁺ T cells.

Also provided, among other things, are technologies for characterizing a HLA-DR CAR as described herein and/or compositions comprising the same. In some embodiments, provided are methods for characterizing binding of a HLA-DR CAR to a T cell of a subject. In some embodiments, provided are methods for characterizing binding of a HLA-DR CAR to a T cell of a subject and/or compositions comprising the same include, for example, ELISA, flow cytometry (e.g., FACs), immunohistochemistry, and/or Biacore binding assays.

The present disclosure provides various technologies related to making or manufacturing HLA-DR CARs and/or T cells comprising HLA-DR CARs as described herein and/or compositions containing the same. The present disclosure provides various technologies related to making or manufacturing nucleic acids encoding HLA-DR CARs and/or T cells comprising nucleic acids encoding HLA-DR CARs as described herein and/or compositions containing the same.

As used in this application, the terms “about” and “approximately” are used as equivalents. Any citations to publications, patents, or patent applications herein are incorporated by reference in their entirety. Any numerals used in this application with or without about/approximately are meant to cover any normal fluctuations appreciated by one of ordinary skill in the relevant art.

Other features, objects, and advantages of the present invention are apparent in the detailed description that follows. It should be understood, however, that the detailed description, while indicating embodiments of the present invention, is given by way of illustration only, not limitation. Various changes and modifications within the scope of the invention will become apparent to those skilled in the art from the detailed description.

### BRIEF DESCRIPTION OF THE DRAWING

The Drawing included herein, which is comprised of the following Figures, is for illustration purposes only and not for limitation. The foregoing and other objects, aspects, features, and advantages of the present disclosure will become more apparent and better understood by referring to the following description taken in conjunction with the accompanying figures in which:

FIG. 1 depicts schematics of (A) an exemplary generic CAR construct with an scFv antigen binding domain and (B) a generalized method for with the overarching steps involved in an autologous CAR T cell therapy.

FIG. 2A depicts a sequence alignment of a polymorphic region of HLA-DR and denotes the epitope for an exemplary HLA-DR antibody agent, MVR.

FIG. 2B depicts binding pattern for an exemplary MVR antibody agent to PBMC cells from different subjects, which were classified as having strong binding affinity (DR^{str}), intermediate binding affinity (DR^{int}) and weak binding affinity (DR^{weak}) as determined by co-staining with CD19:PE or HLA-DR:PE-Cy5 antibody and MVR-scFv with FLAG:APC antibody and flow cytometry.

FIG. 3A depicts second-generation CAR constructions designed using anti-CD19 or an exemplary HLA-DR antibody agent, MVR.

FIG. 3B depicts protein expression in three sets of cells: non-transduced (NT)T, CD19 CAR T, and DR^{weak}MVR CAR T cells, as assessed by western blot analysis to measure CAR protein. Upper bands are CAR protein and lower bands are β-actin. Panel a (top) is a cropped version of the western blot, with the full blot shown directly below (panel b) for reference.

FIG. 4A depicts growth (left panels) and viability (right panels) of NT T, CD19 CAR T, and HLA-DR CAR T cells after activation of DR^{str}, DR^{int}, or DR^{weak} PBMCs. Fold-increases in cell counts (relative to the number on day 0) and viabilities of non-transduced (NT) T, CD19 CAR T, and MVR CAR T cells were measured at the indicated time points. Both CD19 CAR T and MVR CAR T cells were transduced on day 2.

FIG. 4B depicts expression of CAR on NT T, CD19 CAR T, and MVR CAR T cells generated from DR^{str}, DR^{int}, and DR^{weak} PBMCs. Cells were analyzed for CD8 and CAR expression at 13 days post-transduction.

FIG. 5A depicts flow cytometry analysis of CAR and exhaustion marker expression level on day 15. FIG. 5B depicts exemplary pie chart data of the frequency of T cells with multiple exhaustion marker (i.e., LAG-3, Tim-3, CTLA-4, and PD-1) expression measured in FIG. 5A. Each CAR T cell was analyzed by gating on CAR-positive cells. Numbers right side of each color indicates multiplicity of exhaustion markers.

FIG. 6 depicts (a) Proliferation capacity of each CAR T cells measured after activation by DR^{weak}-EBV-LCLs or DR^{str}-EBV-LCLs. CF SE-labeled T cells were co-incubated with each EBV-LCLs at an E:T ratio of 3:1 for 5 days and analyzed by flow cytometry. (b) Pie chart data of the frequency of T cells with multiple marker (i.e., IFN-γ, TNF, IL-2, MIP-1β, and CD107a). Numbers right side of each color indicates multiplicity of markers. (c) Killing efficacy of each CAR T cells against EBV-LCLs. Either DR^{weak}-EBV-LCLs or DR^{str}-EBV-LCLs were co-incubated with each T cells at an indicated E:T ratio for 4 hours. After incubation, induced cytotoxicity was measured to calculate killing efficacy. Each point and error bar indicate mean and SD. Performed in technical duplicate. Representative of two independent experiments. (d) Evaluation of target-specific killing of each CAR T cells by in vitro on-target assay. EBV-LCLs and PBMCs from either DR^{weak} or DR^{str} donor were co-incubated with each CAR T cells at a T cell:EBV-LCL:PBMC ratio of 6:1:1 for 4 hours. After incubation, number of residual viable cells was analyzed by flow cytometry. Each bar and error bar indicate mean and SD. Performed in technical triplicate. Representative of two independent experiments.

FIG. 7A depicts differences in surface CAR expression between CD19 CAR T and MVR CAR T cells. The mean fluorescence intensity (MFI) of the CAR expressed by DR^{weak} MVR CAR T cells was divided by that of CD19 CAR T cells. CD4⁺ or CD8⁺ T cells were analyzed separately. Flow cytometric data from separately generated CAR T cell preparations was used (n=8). Horizontal lines indicate mean. Summary of eight independent experiments.

FIG. 7B depicts lentivirus titer-dependent changes in expression of surface CAR. 293T cells and DR^{weak} T cells were transduced with each CAR vector at various multiplicities of infection, and analyzed for MFI of CAR by flow cytometry. 293T cell lines and DR^{weak} T cells were analyzed at 5 and 13 days post-transduction, respectively.

FIG. 7C depicts DR^{weak} T cells transduced with the CD19 CAR or MVR CAR vector were analyzed for CAR expression at the indicated times post-transduction. Cells were analyzed for CD8 and CAR expression.

FIG. 7D depicts CAR expression analyzed at the mRNA (left) and protein (right) levels by qPCR and western blotting, respectively. Non-transduced (NT) T, CD19 CAR T, and DR^{weak} MVR CAR T cells were subjected to CD4-negative sorting to enrich for CD8⁺ T cells using CD4 microbeads (130-045-101, Miltenyi Biotec, Inc.) and used for analysis. n=3 biological replicates. Mean±s.e.m. Unpaired two-tailed t-test: ns, not significant; ***, p<0.001.

FIG. 8 depicts immunofluorescence staining of NT T, CD19 CAR T, and DR^{weak} MVR CAR T cells.

FIG. 9A depicts target-specific killing of DR^{weak} MVR CAR T cells on day 2 or 12 post-transduction (D2 or D12, respectively). DR^{weak} EBV LCLs were co-incubated with D2 or D12 MVR CAR T cells. After incubation, the number of viable cells was determined and killing efficacy was calculated.

FIG. 9B depicts target-specific killing by each CAR T cell type evaluated with an in vitro on-target killing assay. EBV LCLs and peripheral blood mononuclear cells carrying either DR^{weak} or DR^{str} HLA-DRB1 alleles were co-incubated with NT T, CD19 CAR T, or DR^{weak} MVR CAR T cells. After incubation, the number of viable cells was determined and the killing efficacy was calculated.

FIG. 9C depicts proliferation capacity of T cells measured after activation by DR^{weak} EBV LCLs or DR^{str} EBV LCLs.

FIG. 9D depicts HLA-DR expression in LPS-treated B cells.

FIG. 9E depicts target-specific killing of DR^{weak} MVR CAR T cells on day 2 or 12 post-transduction (referred to as untuned MVR CAR T or MVR CAR T, respectively). DR^{weak} B cells, DR^{str} B cells, and DR^{weak} B cells treated with lipopolysaccharide for 3 days were co-incubated with untuned MVR CAR T or MVR CAR T cells. After incubation, the number of viable cells was determined and the killing efficacy was calculated.

FIG. 9F depicts proportions of B cells and EBV LCLs containing transferred granules after contact with T cells. NT, non-transduced.

FIG. 9G depicts time-lapse analysis of apoptotic EBV LCLs after contact with T cells. EBV LCLs (blue) undergoing apoptosis (red) identified by detecting magenta color (scale bar indicates 250 μm).

FIG. 9H depicts proportions of apoptotic EBV LCLs at indicated time points. Three different areas of each sample were analyzed.

FIG. 9I depicts quantitative molecule analysis of CD19 and HLA-DR on B cell and EBV-LCL surface. Changes in CD19 and HLA-DR counts before and after EBV-transformation were measured by Quantum Simply Cellular microspheres (Bangs Laboratories, Inc.). Paired dots by connected line indicate the same donor (n=6). Red and blue dots respectively indicate DR^{low} and DR^{high} donors used in this study.

FIG. 9J, FIG. 9K and FIG. 9L depict details of an exemplified granule transfer assay.

FIG. 10 depicts (a) the gating strategy for evaluating polyfunctionality of CAR Tcells exemplified herein. CD4⁺ and CD8⁺ T cells were analyzed by gating on carboxy-fluoresceinsuccinimidylester(CF SE)-negative/CD4-positive cells and CF SE-negative/CD4-negative cells, respectively. The expression of each cytokine was determined relative to the expression of T cells stained with isotype control antibodies. (b) Detailed polyfunctionality analysis. Combinations of cytokine-expressing cells were analyzed by Boolean gating.

FIG. 11 depicts an illustrative schematic summary of certain HLA-DR CAR T cells and target cells exemplified in the present disclosure.

FIG. 12A depicts a schematic of a procedure for evaluating EBV LCL suppression in vivo.

FIG. 12B depicts images of mice from an exemplary luciferase activity assay to assess efficacies of DR^{weak} EBV LCL suppression after infusion with non-transduced (NT) T, CD19 CAR T, or DR^{weak} MVR CAR T cells. Luciferase activity in mice grafted with luciferase-labeled DR^{weak} EBV LCLs was measured on 0, 7, 14, 21, and 28 days post-T cell infusion.

FIG. 12C depicts a schematic of a procedure for an in vivo on-target killing assay. Xenografting of D^{weak} B cell/DR^{weak} EBV LCL was followed by infusion with NT T, CD19 CAR T, or DR^{weak} MVR CAR T cells, and subsequent efficacy analysis.

FIG. 12D depicts efficacy of EBV LCL suppression after infusion with each T cell observed for 14 days. Luciferase activity in mice grafted with DR^{weak} B cells and luciferase-labeled DR^{weak} EBV LCLs was measured on −1, 7, and 14 days post-T cell infusion.

FIG. 12E depicts B cell persistence (top panels) in T cell-infused mice on −1, 2, and 7 days post-T cell infusion. Peripheral blood of each mouse was stained with a panel of antibodies and analyzed and plasma IFN-γ levels (bottom panels) measured in mice infused with NT T, CD19 CAR T, or DR^{weak} MVR CAR T cell on −1, 2, and 7 days post-T cell infusion.

FIG. 12F depicts (a, b) gating strategy for analysis of B cells in an in vivo on-target assay. The results of the analysis on the day before T cell infusion are shown in panel a and 2 days post infusion in panel b. Whole blood cells were analyzed for CD3, CD20, CD45, and HLA-DR expression The B-cell population was determined by gating on CD45-positive/CD3-negative/HLA-DR-positive/CD20-positive cells. Mice grafted with only DR^{weak} B cells (b cell only (or DR weak EBV LCLs (tumor only) were also assessed as controls. (c) depicts the expression level of HLA-DR on DR^{weak} B cells in mice infused with non-transduced (NT) T, CD19 CAR T, and DR^{weak} MVR CAR T cells. The mean fluorescence intensity of HLA-DR on B cells is used for comparison.

FIG. 13 depicts expression of HLA-DR on the surface of well-known malignant B cell lines. Cells were analyzed for HLA-DR expression and antibody binding capacity (ABC) is an index of target molecule abundance. The upper and lower dotted lines indicate the average HLA-DR levels of EBV LCLs and B cells, respectively.

FIG. 14 depicts a schematic of a mechanism of EBV LCL-specific killing of MVR CAR T cells. T cells transduced with MVR CAR express CAR on their surface, and MVR CAR is downregulated by the interaction of HLA-DR with HLA-DR CAR (e.g., MVR CAR). Autotuned HLA-DR CAR (e.g., MVR CAR) T cells are desensitized to HLA-DR and exhibit reduced cytotoxicity against normal B cells. EBV-transformed B cells upregulate HLA-DR on their surface and are susceptible to killing by MVR CAR T cells.

FIG. 15 depicts a Venn diagram of certain properties of HLA-DR CART cells of the present disclosure.

### CERTAIN DEFINITIONS

In the description that follows, a number of terms used in biochemistry, molecular biology and immunology are extensively utilized. In order to provide a clearer and consistent understanding of the specification and claims, including the scope to be given such terms, the following definitions are provided.

About: The term “about”, when used herein in reference to a value, refers to a value that is similar, in context to the referenced value. In general, those skilled in the art, familiar with the context, will appreciate the relevant degree of variance encompassed by “about” in that context. For example, in some embodiments, the term “about” may encompass a range of values that within 25%, 20%, 19%, 18%, 17%, 16%, 15%, 14%, 13%, 12%, 11%, 10%, 9%, 8%, 7%, 6%, 5%, 4%, 3%, 2%, 1%, or less of the referred value.

Administration: As used herein, the term “administration” typically refers to the administration of a composition to a subject or system to achieve delivery of an agent that is, or is included in, the composition. Those of ordinary skill in the art will be aware of a variety of routes that may, in appropriate circumstances, be utilized for administration to a subject, for example a human. For example, in some embodiments, administration may be ocular, oral, parenteral, topical, etc.. In some particular embodiments, administration may be bronchial (e.g., by bronchial instillation), buccal, dermal (which may be or comprise, for example, one or more of topical to the dermis, intradermal, interdermal, transdermal, etc), enteral, intra-arterial, intradermal, intragastric, intramedullary, intramuscular, intranasal, intraperitoneal, intrathecal, intravenous, intraventricular, within a specific organ (e. g. intrahepatic), mucosal, nasal, oral, rectal, subcutaneous, sublingual, topical, tracheal (e.g., by intratracheal instillation), vaginal, vitreal, etc. In some embodiments, administration may involve only a single dose. In some embodiments, administration may involve application of a fixed number of doses. In some embodiments, administration may involve dosing that is intermittent (e.g., a plurality of doses separated in time) and/or periodic (e.g., individual doses separated by a common period of time) dosing. In some embodiments, administration may involve continuous dosing (e.g., perfusion) for at least a selected period of time.

Affinity: As is known in the art, “affinity” is a measure of the tightness with a particular ligand binds to its partner. Affinities can be measured in different ways. In some embodiments, affinity is measured by a quantitative assay. In some such embodiments, binding partner concentration may be fixed to be in excess of ligand concentration so as to mimic physiological conditions. Alternatively or additionally, in some embodiments, binding partner concentration and/or ligand concentration may be varied. In some such embodiments, affinity may be compared to a reference under comparable conditions (e.g., concentrations).

Animal: as used herein refers to any member of the animal kingdom. In some embodiments, “animal” refers to humans, of either sex and at any stage of development. In some embodiments, “animal” refers to non-human animals, at any stage of development. In certain embodiments, the non-human animal is a mammal (e.g., a rodent, a mouse, a rat, a rabbit, a monkey, a dog, a cat, a sheep, cattle, a primate, and/or a pig). In some embodiments, animals include, but are not limited to, mammals, birds, reptiles, amphibians, fish, insects, and/or worms. In some embodiments, an animal may be a transgenic animal, genetically engineered animal, and/or a clone.

Antibody agent: As used herein, the term “antibody agent” refers to an agent that specifically binds to a particular antigen. In some embodiments, the term encompasses any polypeptide or polypeptide complex that includes immunoglobulin structural elements sufficient to confer specific binding. Exemplary antibody agents include, but are not limited to monoclonal antibodies, polyclonal antibodies, and fragments thereof. In some embodiments, an antibody agent may include one or more sequence elements are humanized, primatized, chimeric, etc, as is known in the art. In many embodiments, the term “antibody agent” is used to refer to one or more of the art-known or developed constructs or formats for utilizing antibody structural and functional features in alternative presentation. For example, embodiments, an antibody agent utilized in accordance with the present invention is in a format selected from, but not limited to, intact IgA, IgG, IgE or IgM antibodies; bi- or multi-specific antibodies (e.g., Zybodies®, etc); antibody fragments such as Fab fragments, Fab′ fragments, F(ab′)2 fragments, Fd′ fragments, Fd fragments, and isolated CDRs or sets thereof single chain Fvs; polypeptide-Fc fusions; single domain antibodies (e.g., shark single domain antibodies such as IgNAR or fragments thereof); cameloid antibodies; masked antibodies (e.g., Probodies®); Small Modular ImmunoPharmaceuticals (“SMIPs™”); single chain or Tandem diabodies (TandAb®); VHHs; Anticalins®; Nanobodies® minibodies; BiTE®s; ankyrin repeat proteins or DARPINs®; Avimers®; DARTs; TCR-like antibodies; Adnectins®; Affilins®; Trans-Bodies®; Affibodies®; TrimerX®; MicroProteins; Fynomers®, Centyrins®; and KALBITOR®s. In some embodiments, an antibody agent may lack a covalent modification (e.g., attachment of a glycan) that it would have if produced naturally. In some embodiments, an antibody agent may contain a covalent modification (e.g., attachment of a glycan, a payload [e.g., a detectable moiety, a therapeutic moiety, a catalytic moiety, etc], or other pendant group [e.g., poly-ethylene glycol, etc.]. In many embodiments, an antibody agent is or comprises a polypeptide whose amino acid sequence includes one or more structural elements recognized by those skilled in the art as a complementarity determining region (CDR); in some embodiments an antibody agent is or comprises a polypeptide whose amino acid sequence includes at least one CDR (e.g., at least one heavy chain CDR and/or at least one light chain CDR) that is substantially identical to one found in a reference antibody. In some embodiments an included CDR is substantially identical to a reference CDR in that it is either identical in sequence or contains between 1-5 amino acid substitutions as compared with the reference CDR. In some embodiments an included CDR is substantially identical to a reference CDR in that it shows at least 85%, 86%, 87%, 88%, 89%, 90%, 91%, 92%, 93%, 94%, 95%, 96%, 97%, 98%, 99%, or 100% sequence identity with the reference CDR. In some embodiments an included CDR is substantially identical to a reference CDR in that it shows at least 96%, 96%, 97%, 98%, 99%, or 100% sequence identity with the reference CDR. In some embodiments an included CDR is substantially identical to a reference CDR in that at least one amino acid within the included CDR is deleted, added, or substituted as compared with the reference CDR but the included CDR has an amino acid sequence that is otherwise identical with that of the reference CDR. In some embodiments an included CDR is substantially identical to a reference CDR in that 1-5 amino acids within the included CDR are deleted, added, or substituted as compared with the reference CDR but the included CDR has an amino acid sequence that is otherwise identical to the reference CDR. In some embodiments an included CDR is substantially identical to a reference CDR in that at least one amino acid within the included CDR is substituted as compared with the reference CDR but the included CDR has an amino acid sequence that is otherwise identical with that of the reference CDR. In some embodiments an included CDR is substantially identical to a reference CDR in that 1-5 amino acids within the included CDR are deleted, added, or substituted as compared with the reference CDR but the included CDR has an amino acid sequence that is otherwise identical to the reference CDR. In some embodiments, an antibody agent is or comprises a polypeptide whose amino acid sequence includes structural elements recognized by those skilled in the art as an immunoglobulin variable domain. In some embodiments, an antibody agent is a polypeptide protein having a binding domain which is homologous or largely homologous to an immunoglobulin-binding domain. In some embodiments, an antibody agent is or comprises at least a portion of a chimeric antigen receptor (CAR).

Antigen: The term “antigen”, as used herein, refers to an agent that binds to an antibody agent. In some embodiments, an antigen binds to an antibody agent and may or may not induce a particular physiological response in an organism. In general, an antigen may be or include any chemical entity such as, for example, a small molecule, a nucleic acid, a polypeptide, a carbohydrate, a lipid, a polymer (including biologic polymers [e.g., nucleic acid and/or amino acid polymers] and polymers other than biologic polymers [e.g., other than a nucleic acid or amino acid polymer]) etc. In some embodiments, an antigen is or comprises a polypeptide. In some embodiments, an antigen is or comprises a glycan. Those of ordinary skill in the art will appreciate that, in general, an antigen may be provided in isolated or pure form, or alternatively may be provided in crude form (e.g., together with other materials, for example in an extract such as a cellular extract or other relatively crude preparation of an antigen-containing source). In some certain embodiments, an antigen is present in a cellular context (e.g., an antigen is expressed on the surface of a cell or expressed in a cell). In some embodiments, an antigen is a recombinant antigen.

Antigen binding domain: As used herein, refers to an antibody agent or portion thereof that specifically binds to a target moiety or entity. Typically, the interaction between an antigen binding domain and its target is non-covalent. In some embodiments, a target moiety or entity can be of any chemical class including, for example, a carbohydrate, a lipid, a nucleic acid, a metal, a polypeptide, or a small molecule. In some embodiments, an antigen binding domain may be or comprise a polypeptide (or complex thereof). In some embodiments, an antigen binding domain is part of a fusion polypeptide. In some embodiments, an antigen binding domain is part of a chimeric antigen recept (CAR).

Associated with: Two events or entities are “associated” with one another, as that term is used herein, if the presence, level and/or form of one is correlated with that of the other. For example, a particular entity (e.g., polypeptide, genetic signature, metabolite, microbe, etc) is considered to be associated with a particular disease, disorder, or condition, if its presence, level and/or form correlates with incidence of and/or susceptibility to the disease, disorder, or condition (e.g., across a relevant population). In some embodiments, two or more entities are physically “associated” with one another if they interact, directly or indirectly, so that they are and/or remain in physical proximity with one another. In some embodiments, two or more entities that are physically associated with one another are covalently linked to one another; in some embodiments, two or more entities that are physically associated with one another are not covalently linked to one another but are non-covalently associated, for example by means of hydrogen bonds, van der Waals interaction, hydrophobic interactions, magnetism, and combinations thereof.

Binding: It will be understood that the term “binding”, as used herein, typically refers to a non-covalent association between or among two or more entities. “Direct” binding involves physical contact between entities or moieties; indirect binding involves physical interaction by way of physical contact with one or more intermediate entities. Binding between two or more entities can typically be assessed in any of a variety of contexts—including where interacting entities or moieties are studied in isolation or in the context of more complex systems (e.g., while covalently or otherwise associated with a carrier entity and/or in a biological system or cell).

Cancer: The terms “cancer”, “malignancy”, “neoplasm”, “tumor”, and “carcinoma”, are used herein to refer to cells that exhibit relatively abnormal, uncontrolled, and/or autonomous growth, so that they exhibit an aberrant growth phenotype characterized by a significant loss of control of cell proliferation. In some embodiments, a tumor may be or comprise cells that are precancerous (e.g., benign), malignant, pre-metastatic, metastatic, and/or non-metastatic. The present disclosure specifically identifies certain cancers to which its teachings may be particularly relevant. In some embodiments, a relevant cancer may be characterized by a solid tumor. In some embodiments, a relevant cancer may be characterized by a hematologic tumor. In general, examples of different types of cancers known in the art include, for example, hematopoietic cancers including leukemias, lymphomas (Hodgkin's and non-Hodgkin's), myelomas and myeloproliferative disorders; sarcomas, melanomas, adenomas, carcinomas of solid tissue, squamous cell carcinomas of the mouth, throat, larynx, and lung, liver cancer, genitourinary cancers such as prostate, cervical, bladder, uterine, and endometrial cancer and renal cell carcinomas, bone cancer, pancreatic cancer, skin cancer, cutaneous or intraocular melanoma, cancer of the endocrine system, cancer of the thyroid gland, cancer of the parathyroid gland, head and neck cancers, breast cancer, gastro-intestinal cancers and nervous system cancers, benign lesions such as papillomas, and the like. In some embodiments, a cancer is a hematologic cancer. Hematological cancers can include, for example, acute leukemias including but not limited to B-cell acute lymphoid leukemia (“BALL”), T-cell acute lymphoid leukemia (“TALL”), acute lymphoid leukemia (ALL); one or more chronic leukemias including but not limited to chronic myelogenous leukemia (CIVIL), chronic lymphocytic leukemia (CLL); additional hematologic cancers or hematologic conditions including, but not limited to B cell prolymphocytic leukemia, blastic plasmacytoid dendritic cell neoplasm, Burkitt's lymphoma, diffuse large B cell lymphoma, follicular lymphoma, hairy cell leukemia, small cell- or a large cell-follicular lymphoma, malignant lymphoproliferative conditions, MALT lymphoma, mantle cell lymphoma, Marginal zone lymphoma, multiple myeloma, myelodysplasia and myelodysplastic syndrome, non-Hodgkin's lymphoma, plasmablastic lymphoma, plasmacytoid dendritic cell neoplasm, Waldenstrom macroglobulinemia, and “preleukemia” which are a diverse collection of hematological conditions united by ineffective production (or dysplasia) of myeloid blood cells, and to atypical and/or non-classical cancers, malignancies, and precancerous conditions or proliferative diseases.

CDR: as used herein, refers to a complementarity determining region within a variable region of an antibody agent. There are three CDRs in each of the variable regions of the heavy chain and the light chain, which are designated CDR1, CDR2 and CDR3, for each of the variable regions. A “set of CDRs” or “CDR set” refers to a group of three or six CDRs that occur in either a single variable region capable of binding the antigen or the CDRs of cognate heavy and light chain variable regions capable of binding the antigen. Certain systems have been established in the art for defining CDR boundaries (e.g., Kabat, Chothia, etc.); those skilled in the art appreciate the differences between and among these systems and are capable of understanding CDR boundaries to the extent required to understand and to practice the claimed invention.

Chemotherapeutic Agent: The term “chemotherapeutic agent”, has used herein has its art-understood meaning referring to one or more pro-apoptotic, cytostatic and/or cytotoxic agents, for example specifically including agents utilized and/or recommended for use in treating one or more diseases, disorders or conditions associated with undesirable cell proliferation. In many embodiments, chemotherapeutic agents are useful in the treatment of cancer. In some embodiments, a chemotherapeutic agent may be or comprise one or more alkylating agents, one or more anthracyclines, one or more cytoskeletal disruptors (e.g. microtubule targeting agents such as taxanes, maytansine and analogs thereof, of), one or more epothilones, one or more histone deacetylase inhibitors HDACs), one or more topoisomerase inhibitors (e.g., inhibitors of topoisomerase I and/or topoisomerase II), one or more kinase inhihitors, one or more nucleotide analogs or nucleotide precursor analogs, one or more peptide antibiotics, one or more platinum-based agents, one or more retinoids, one or more vinca alkaloids, and/or one or more analogs of one or more of the following (i.e., that share a relevant anti-proliferative activity). In some particular embodiments, a chemotherapeutic agent may be or comprise one or more of Actinomycin, All-trans retinoic acid, an Auiristatin, Azacitidine, Azathioprine, Bleomycin, Bortezomib, Carboplatin, Capecitabine, Cisplatin, Chlorambucil, Cyclophosphamide, Curcumin, Cytarabine, Daunorubicin, Docetaxel, Doxifluridine, Doxorubicin, Epirubicin, Epothilone, Etoposide, Fluorouracil, Gemcitabine, Hydroxyurea, Idarubicin, Imatinib, Irinotecan, Maytansine and/or analogs thereof (e.g. DM1) Mechlorethamine, Mercaptopurine, Methotrexate, Mitoxantrone, a Maytansinoid, Oxaliplatin, Paclitaxel, Pemetrexed, Teniposide, Tioguanine, Topotecan, Valrubicin, Vinblastine, Vincristine, Vindesine, Vinorelbine, and combinations thereof. In some embodiments, a chemotherapeutic agent may be utilized in the context of an antibody-drug conjugate. In some embodiments, a chemotherapeutic agent is one found in an antibody-drug conjugate selected from the group consisting of: hLL1-doxorubicin, hRS7-SN-38, hMN-14-SN-38, hLL2-SN-38, hA20-SN-38, hPAM4-SN-38, hLL1-SN-38, hRS7-Pro-2-P-Dox, hMN-14-Pro-2-P-Dox, hLL2-Pro-2-P-Dox, hA20-Pro-2-P-Dox, hPAM4-Pro-2-P-Dox, hLL1-Pro-2-P-Dox, P4/D10-doxorubicin, gemtuzumab ozogamicin, brentuximab vedotin, trastuzumab emtansine, inotuzumab ozogamicin, glembatumomab vedotin, SAR3419, SAR566658, BIIB015, BT062, SGN-75, SGN-CD19A, AMG-172, AMG-595, BAY-94-9343, ASG-5ME, ASG-22ME, ASG-16M8F, MDX-1203, MLN-0264, anti-PSMA ADC, RG-7450, RG-7458, RG-7593, RG-7596, RG-7598, RG-7599, RG-7600, RG-7636, ABT-414, IMGN-853, IMGN-529, vorsetuzumab mafodotin, and lorvotuzumab mertansine.

Combination therapy: As used herein, the term “combination therapy” refers to those situations in which a subject is simultaneously exposed to two or more therapeutic regimens (e.g., two or more therapeutic agents). In some embodiments, the two or more therapeutic regimens may be administered simultaneously. In some embodiments, the two or more therapeutic regimens may be administered sequentially (e.g., a first regimen administered prior to administration of any doses of a second regimen). In some embodiments, the two or more therapeutic regimens are administered in overlapping dosing regimens. In some embodiments, administration of combination therapy may involve administration of one or more therapeutic agents or modalities to a subject receiving the other agent(s) or modality.

Engineered: In general, the term “engineered” refers to the aspect of having been manipulated by the hand of man. For example, a polypeptide is considered to be “engineered” when the polypeptide sequence manipulated by the hand of man. For example, in some embodiments of the present invention, an engineered polypeptide comprises a sequence that includes one or more amino acid mutations, deletions and/or insertions that have been introduced by the hand of man into a reference polypeptide sequence. In some embodiments, an engineered polypeptide includes a polypeptide that has been fused (i.e., covalently linked) to one or more additional polypeptides by the hand of man, to form a fusion polypeptide that would not naturally occur in vivo. Comparably, a cell or organism is considered to be “engineered” if it has been manipulated so that its genetic information is altered (e.g., new genetic material not previously present has been introduced, for example by transformation, mating, somatic hybridization, transfection, transduction, or other mechanism, or previously present genetic material is altered or removed, for example by substitution or deletion mutation, or by mating protocols). As is common practice and is understood by those in the art, derivatives and/or progeny of an engineered polypeptide or cell are typically still referred to as “engineered” even though the actual manipulation was performed on a prior entity.

Epitope: as used herein, includes any moiety that is specifically recognized by an immunoglobulin (e.g., antibody agent or receptor) binding component. In some embodiments, an epitope is comprised of a plurality of chemical atoms or groups on an antigen. In some embodiments, such chemical atoms or groups are surface-exposed when the antigen adopts a relevant three-dimensional conformation. In some embodiments, such chemical atoms or groups are physically near to each other in space when the antigen adopts such a conformation. In some embodiments, at least some such chemical atoms are groups are physically separated from one another when the antigen adopts an alternative conformation (e.g., is linearized).

Ex vivo: as used herein refers to biologic events that occur outside of the context of a multicellular organism. For example, in the context of cell-based systems, the term may be used to refer to events that occur among a population of cells (e.g., cell proliferation, cytokine secretion, etc.) in an artificial environment.

Framework or framework region: as used herein, refers to the sequences of a variable region minus the CDRs. Because a CDR sequence can be determined by different systems, likewise a framework sequence is subject to correspondingly different interpretations. The six CDRs divide the framework regions on the heavy and light chains into four sub-regions (FR1, FR2, FR3 and FR4) on each chain, in which CDR1 is positioned between FR1 and FR2, CDR2 between FR2 and FR3, and CDR3 between FR3 and FR4. Without specifying the particular sub-regions as FR1, FR2, FR3 or FR4, a framework region, as referred by others, represents the combined FRs within the variable region of a single, naturally occurring immunoglobulin chain. As used herein, a FR represents one of the four sub-regions, FR1, for example, represents the first framework region closest to the amino terminal end of the variable region and 5′ with respect to CDR1, and FRs represents two or more of the sub-regions constituting a framework region.

In vitro: The term “in vitro” as used herein refers to events that occur in an artificial environment, e.g., in a test tube or reaction vessel, in cell culture, etc., rather than within a multi-cellular organism.

In vivo: as used herein refers to events that occur within a multi-cellular organism, such as a human and a non-human animal. In the context of cell-based systems, the term may be used to refer to events that occur within a living cell (as opposed to, for example, in vitro systems).

Isolated: as used herein, refers to a substance and/or entity that has been (1) separated from at least some of the components with which it was associated when initially produced (whether in nature and/or in an experimental setting), and/or (2) designed, produced, prepared, and/or manufactured by the hand of man. Isolated substances and/or entities may be separated from about 10%, about 20%, about 30%, about 40%, about 50%, about 60%, about 70%, about 80%, about 90%, about 91%, about 92%, about 93%, about 94%, about 95%, about 96%, about 97%, about 98%, about 99%, or more than about 99% of the other components with which they were initially associated. In some embodiments, isolated agents are about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components. In some embodiments, as will be understood by those skilled in the art, a substance may still be considered “isolated” or even “pure”, after having been combined with certain other components such as, for example, one or more carriers or excipients (e.g., buffer, solvent, water, etc.); in such embodiments, percent isolation or purity of the substance is calculated without including such carriers or excipients. To give but one example, in some embodiments, a biological polymer such as a polypeptide or polynucleotide that occurs in nature is considered to be “isolated” when, a) by virtue of its origin or source of derivation is not associated with some or all of the components that accompany it in its native state in nature; b) it is substantially free of other polypeptides or nucleic acids of the same species from the species that produces it in nature; c) is expressed by or is otherwise in association with components from a cell or other expression system that is not of the species that produces it in nature. Thus, for instance, in some embodiments, a polypeptide that is chemically synthesized or is synthesized in a cellular system different from that which produces it in nature is considered to be an “isolated” polypeptide. Alternatively or additionally, in some embodiments, a polypeptide that has been subjected to one or more purification techniques may be considered to be an “isolated” polypeptide to the extent that it has been separated from other components a) with which it is associated in nature; and/or b) with which it was associated when initially produced.

K_{D}: as used herein, refers to the dissociation constant of a binding agent (e.g., an antibody agent or binding component thereof) from a complex with its partner (e.g., the epitope to which the antibody agent or binding component thereof binds).

Operably linked: as used herein, refers to a juxtaposition wherein the components described are in a relationship permitting them to function in their intended manner. A control element “operably linked” to a functional element is associated in such a way that expression and/or activity of the functional element is achieved under conditions compatible with the control element. In some embodiments, “operably linked” control elements are contiguous (e.g., covalently linked) with the coding elements of interest; in some embodiments, control elements act in trans to or otherwise at a from the functional element of interest.

Pharmaceutical composition: As used herein, the term “pharmaceutical composition” refers to a composition in which an active agent is formulated together with one or more pharmaceutically acceptable carriers. In some embodiments, the composition is suitable for administration to a human or animal subject. In some embodiments, the active agent is present in unit dose amount appropriate for administration in a therapeutic regimen that shows a statistically significant probability of achieving a predetermined therapeutic effect when administered to a relevant population.

Polypeptide: The term “polypeptide”, as used herein, generally has its art-recognized meaning of a polymer of at least three amino acids. Those of ordinary skill in the art will appreciate that the term “polypeptide” is intended to be sufficiently general as to encompass not only polypeptides having a complete sequence recited herein, but also to encompass polypeptides that represent functional fragments (i.e., fragments retaining at least one activity) of such complete polypeptides. Moreover, those of ordinary skill in the art understand that protein sequences generally tolerate some substitution without destroying activity. Thus, any polypeptide that retains activity and shares at least about 30-40% overall sequence identity, often greater than about 50%, 60%, 70%, or 80%, and further usually including at least one region of much higher identity, often greater than 90% or even 95%, 96%, 97%, 98%, or 99% in one or more highly conserved regions, usually encompassing at least 3-4 and often up to 20 or more amino acids, with another polypeptide of the same class, is encompassed within the relevant term “polypeptide” as used herein. Polypeptides may contain L-amino acids, D-amino acids, or both and may contain any of a variety of amino acid modifications or analogs known in the art. Useful modifications include, e.g., terminal acetylation, amidation, methylation, etc. In some embodiments, proteins may comprise natural amino acids, non-natural amino acids, synthetic amino acids, and combinations thereof. The term “peptide” is generally used to refer to a polypeptide having a length of less than about 100 amino acids, less than about 50 amino acids, less than 20 amino acids, or less than 10 amino acids. In some embodiments, proteins are antibody agents, antibody fragments, biologically active portions thereof, and/or characteristic portions thereof.

Prevent or prevention: as used herein when used in connection with the occurrence of a disease, disorder, and/or condition, refers to reducing the risk of developing the disease, disorder and/or condition and/or to delaying onset and/or severity of one or more characteristics or symptoms of the disease, disorder or condition. In some embodiments, prevention is assessed on a population basis such that an agent is considered to “prevent” a particular disease, disorder or condition if a statistically significant decrease in the development, frequency, and/or intensity of one or more symptoms of the disease, disorder or condition is observed in a population susceptible to the disease, disorder, or condition.

Recombinant: as used herein, is intended to refer to polypeptides that are designed, engineered, prepared, expressed, created, manufactured, and/or or isolated by recombinant means, such as polypeptides expressed using a recombinant expression vector transfected into a host cell; polypeptides isolated from a recombinant, combinatorial human polypeptide library; polypeptides isolated from an animal (e.g., a mouse, rabbit, sheep, fish, etc) that is transgenic for or otherwise has been manipulated to express a gene or genes, or gene components that encode and/or direct expression of the polypeptide or one or more component(s), portion(s), element(s), or domain(s) thereof; and/or polypeptides prepared, expressed, created or isolated by any other means that involves splicing or ligating selected nucleic acid sequence elements to one another, chemically synthesizing selected sequence elements, and/or otherwise generating a nucleic acid that encodes and/or directs expression of the polypeptide or one or more component(s), portion(s), element(s), or domain(s) thereof. In some embodiments, one or more of such selected sequence elements is found in nature. In some embodiments, one or more of such selected sequence elements is designed in silico. In some embodiments, one or more such selected sequence elements results from mutagenesis (e.g., in vivo or in vitro) of a known sequence element, e.g., from a natural or synthetic source such as, for example, in the germline of a source organism of interest (e.g., of a human, a mouse, etc).

Specific binding: As used herein, the term “specific binding” refers to an ability to discriminate between possible binding partners in the environment in which binding is to occur. A binding agent that interacts with one particular target when other potential targets are present is said to “bind specifically” to the target with which it interacts. In some embodiments, specific binding is assessed by detecting or determining degree of association between the binding agent and its partner; in some embodiments, specific binding is assessed by detecting or determining degree of dissociation of a binding agent-partner complex; in some embodiments, specific binding is assessed by detecting or determining ability of the binding agent to compete an alternative interaction between its partner and another entity. In some embodiments, specific binding is assessed by performing such detections or determinations across a range of concentrations.

Subject: As used herein, the term “subject” refers an organism, typically a mammal (e.g., a human, in some embodiments including prenatal human forms). In some embodiments, a subject is suffering from a relevant disease, disorder or condition. In some embodiments, a subject is susceptible to a disease, disorder, or condition. In some embodiments, a subject displays one or more symptoms or characteristics of a disease, disorder or condition. In some embodiments, a subject does not display any symptom or characteristic of a disease, disorder, or condition. In some embodiments, a subject is someone with one or more features characteristic of susceptibility to or risk of a disease, disorder, or condition. In some embodiments, a subject is a patient. In some embodiments, a subject is an individual to whom diagnosis and/or therapy is and/or has been administered.

Therapeutic agent: As used herein, the phrase “therapeutic agent” in general refers to any agent that elicits a desired pharmacological effect when administered to an organism. In some embodiments, an agent is considered to be a therapeutic agent if it demonstrates a statistically significant effect across an appropriate population. In some embodiments, the appropriate population may be a population of model organisms. In some embodiments, an appropriate population may be defined by various criteria, such as a certain age group, gender, genetic background, preexisting clinical conditions, etc. In some embodiments, a therapeutic agent is a substance that can be used to alleviate, ameliorate, relieve, inhibit, prevent, delay onset of, reduce severity of, and/or reduce incidence of one or more symptoms or features of a disease, disorder, and/or condition. In some embodiments, a “therapeutic agent” is an agent that has been or is required to be approved by a government agency before it can be marketed for administration to humans. In some embodiments, a “therapeutic agent” is an agent for which a medical prescription is required for administration to humans.

Therapeutically Effective Amount: As used herein, the term “therapeutically effective amount” means an amount that is sufficient, when administered to a population suffering from or susceptible to a disease, disorder, and/or condition in accordance with a therapeutic dosing regimen, to treat the disease, disorder, and/or condition. In some embodiments, a therapeutically effective amount is one that reduces the incidence and/or severity of, stabilizes one or more characteristics of, and/or delays onset of, one or more symptoms of the disease, disorder, and/or condition. Those of ordinary skill in the art will appreciate that the term “therapeutically effective amount” does not in fact require successful treatment be achieved in a particular individual. Rather, a therapeutically effective amount may be that amount that provides a particular desired pharmacological response in a significant number of subjects when administered to patients in need of such treatment. For example, in some embodiments, term “therapeutically effective amount”, refers to an amount which, when administered to an individual in need thereof in the context of inventive therapy, will block, stabilize, attenuate, or reverse a cancer-supportive process occurring in said individual, or will enhance or increase a cancer-suppressive process in said individual. In the context of cancer treatment, a “therapeutically effective amount” is an amount which, when administered to an individual diagnosed with a cancer, will prevent, stabilize, inhibit, or reduce the further development of cancer in the individual. A particularly preferred “therapeutically effective amount” of a composition described herein reverses (in a therapeutic treatment) the development of a malignancy such as a pancreatic carcinoma or helps achieve or prolong remission of a malignancy. A therapeutically effective amount administered to an individual to treat a cancer in that individual may be the same or different from a therapeutically effective amount administered to promote remission or inhibit metastasis. As with most cancer therapies, the therapeutic methods described herein are not to be interpreted as, restricted to, or otherwise limited to a “cure” for cancer; rather the methods of treatment are directed to the use of the described compositions to “treat” a cancer, i.e., to effect a desirable or beneficial change in the health of an individual who has cancer. Such benefits are recognized by skilled healthcare providers in the field of oncology and include, but are not limited to, a stabilization of patient condition, a decrease in tumor size (tumor regression), an improvement in vital functions (e.g., improved function of cancerous tissues or organs), a decrease or inhibition of further metastasis, a decrease in opportunistic infections, an increased survivability, a decrease in pain, improved motor function, improved cognitive function, improved feeling of energy (vitality, decreased malaise), improved feeling of well-being, restoration of normal appetite, restoration of healthy weight gain, and combinations thereof. In addition, regression of a particular tumor in an individual (e.g., as the result of treatments described herein) may also be assessed by taking samples of cancer cells from the site of a tumor such as a pancreatic adenocarcinoma (e.g., over the course of treatment) and testing the cancer cells for the level of metabolic and signaling markers to monitor the status of the cancer cells to verify at the molecular level the regression of the cancer cells to a less malignant phenotype. For example, tumor regression induced by employing the methods of this invention would be indicated by finding a decrease in any of the pro-angiogenic markers discussed above, an increase in anti-angiogenic markers described herein, the normalization (i.e., alteration toward a state found in normal individuals not suffering from cancer) of metabolic pathways, intercellular signaling pathways, or intracellular signaling pathways that exhibit abnormal activity in individuals diagnosed with cancer. Those of ordinary skill in the art will appreciate that, in some embodiments, a therapeutically effective amount may be formulated and/or administered in a single dose. In some embodiments, a therapeutically effective amount may be formulated and/or administered in a plurality of doses, for example, as part of a dosing regimen.

Variant: As used herein in the context of molecules, e.g., nucleic acids, proteins, or small molecules, the term “variant” refers to a molecule that shows significant structural identity with a reference molecule but differs structurally from the reference molecule, e.g., in the presence or absence or in the level of one or more chemical moieties as compared to the reference entity. In some embodiments, a variant also differs functionally from its reference molecule. In general, whether a particular molecule is properly considered to be a “variant” of a reference molecule is based on its degree of structural identity with the reference molecule. As will be appreciated by those skilled in the art, any biological or chemical reference molecule has certain characteristic structural elements. A variant, by definition, is a distinct molecule that shares one or more such characteristic structural elements but differs in at least one aspect from the reference molecule. To give but a few examples, a polypeptide may have a characteristic sequence element comprised of a plurality of amino acids having designated positions relative to one another in linear or three-dimensional space and/or contributing to a particular structural motif and/or biological function; a nucleic acid may have a characteristic sequence element comprised of a plurality of nucleotide residues having designated positions relative to on another in linear or three-dimensional space. In some embodiments, a variant polypeptide or nucleic acid may differ from a reference polypeptide or nucleic acid as a result of one or more differences in amino acid or nucleotide sequence. In some embodiments, a variant polypeptide or nucleic acid shows an overall sequence identity with a reference polypeptide or nucleic acid that is at least 85%, 86%, 87%, 88%, 89%, 90%, 91%, 92%, 93%, 94%, 95%, 96%, 97%, or 99%. In some embodiments, a variant polypeptide or nucleic acid does not share at least one characteristic sequence element with a reference polypeptide or nucleic acid. In some embodiments, a reference polypeptide or nucleic acid has one or more biological activities. In some embodiments, a variant polypeptide or nucleic acid shares one or more of the biological activities of the reference polypeptide or nucleic acid.

Vector: as used herein, refers to a nucleic acid molecule capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “expression vectors.” Standard techniques may be used for recombinant DNA, oligonucleotide synthesis, and tissue culture and transformation (e.g., electroporation, lipofection). Enzymatic reactions and purification techniques may be performed according to manufacturer's specifications or as commonly accomplished in the art or as described herein. The foregoing techniques and procedures may be generally performed according to conventional methods well known in the art and as described in various general and more specific references that are cited and discussed throughout the present specification. See e.g., Sambrook et al., Molecular Cloning: A Laboratory Manual 2^{nd} ed., Cold Spring Harbor Laboratory Press, Cold Spring Harbor, N.Y. (1989)), which is incorporated herein by reference for any purpose.

### DETAILED DESCRIPTION OF EXEMPLARY EMBODIMENTS

The present disclosure relates, inter alia, to engineered T cells that express a chimeric antigen receptor (CAR) that includes a HLA-DR antigen binding domain, as well as methods of making and using the same.

T cells engineered with chimeric antigen receptors (CAR T cells) have great therapeutic potential for treating cancers. For example, recent clinical trials of a CD19-targeted CAR-transduced T cell (CD19-CAR T cell) against hematologic malignancies showed a strong effect of CART technology. (Kochenderfer, J. N. et al. (2010) Blood 116: 4099-4102; Porter, D. L., et al. (2011) N. Engl. J. Med. 365: 725-733; Grupp, S. A. et al. (2013) N. Engl. 1 Med. 368: 1509-1518; Kochenderfer, J. N. et al. (2015) J. Clin. Oncol. 33: 540-549; Brown, C. E. et al. (2016) N. Engl. J. Med. 375: 2561-2569). The clinical success of CART is attributed, at least in part, to the fusion structure of the CAR, which is made by artificially combining a high-affinity antigen-binding domain with multiple signaling domains (Maus, M. V. et al. (2014) Blood 123: 2625-2635; van der Stegen, S. J. et al. (2015) Nat. Rev. Drug Discov. 14: 499-509).

However, the impressive outcomes of CAR T therapies have been frequently accompanied by severe side effects, such as cytokine release syndrome (CRS) and B cell aplasia in CD19-CAR T cell-treated patients. (Kalos, M., et al. (2011) Sci. Transl. Med. 3: 95ra73; Davila, M. L., et al. (2014) Sci. Transl. Med. 6: 224ra225). Thus there is an unmet need to develop novel CAR T strategies that reduce and/or alleviate the associated side effects.

CARs frequently target antigens that are not exclusively expressed on malignant cells, but also expressed on normal cells, and in some cases target antigens on T cells themselves. These properties of CARs differ from T cell receptors (TCRs), which are natural antigen receptors for T cells, that typically exhibit low affinity and recognize antigens rarely expressed on normal cells. Despite these differences, some properties of CARs are shared with TCRs.

One shared property of both CARs and TCRs is that both types of receptors can be subject to receptor downregulation. For example, TCRs are rapidly downregulated after antigen recognition to limit excess signaling to maintain signal integrity (Viola, A. & Lanzavecchia, A. (1996) Science 273: 104-106; Baniyash, M. (2004) Nat. Rev. Immunol. 4: 675-687). Similarly, antigen recognition by CARs is often immediately followed by CAR downregulation, which affects subsequent antigen recognition and function (Caruso, H. G. et al. (2015) Cancer Res. 75: 3505-3518; Eyquem, J. et al. (2017) Nature 543: 113-117). These receptor downregulation events can occur within hours and recovery can be on the order of days. In contrast to short-term downregulation, long-term downregulation of TCRs was reported by Gallegos et al. (2016) Nat. Immunol. 17: 379-386. This study demonstrated that certain continuous TCR-target interactions could induce long-term TCR downregulation, which could be sustained for over 50 days. The extent of downregulation in this study correlated with TCR-target affinity and, importantly, resulted in an eventual increase in the overall immune-activation threshold. This phenomenon represents a mechanism by which T cells can tune antigen sensitivity and manage the extent of the immune response at a macro level.

For CAR T cells, Caruso et al. and Liu et al. have demonstrated that certain CARs of low affinity can sensitize T cells to distinguish certain target cells of high antigen density from low (Caruso, H. G., et al. (2015) Cancer Res. 75: 3505-3518; Liu, X., et al. (2015) Cancer Res. 75: 3596-3607). These studies suggested a CAR design strategy that targets tumor antigens which are specifically upregulated in malignant cells. However, long-term CAR downregulation and subsequent functional changes induced by continuous target recognition have not been widely investigated.

While receptor downregulation is observed in both CARs and TCRs, the specific binding characteristics of CARs may result in a distinctive functional consequence known as “fratricide”, which is T cell death induced by neighboring CAR T cells due to targeting of the antigen expressed on T cells. Interestingly, the extent of fratricide is not the same for all CAR constructs. For example, fratricide is transient in CD5-targeted CAR T cells, as they expand normally for several weeks. Mamonkin, M., et al. (2015) Blood 126: 983-992). In contrast, fratricide seriously damages CD7-targeted CAR T cells, resulting in unviability. (Gomes-Silva, D. et al. (2017) Blood 130: 285-296). However, the conditions that allow the extent of fratricide to be tolerable are not well-defined.

The present disclosure provides the insight that HLA-DR-targeted CAR T cells can continuously recognize HLA-DR on neighboring CAR T cells and induce fratricide and CAR downregulation. The present disclosure encompasses a recognition that HLA-DR-targeted CARs that recognizes a polymorphic region of HLA-DR can recognize T cells with different HLA-DRB1 alleles with varying affinities. Moreover, the present disclosure also encompasses a recognition that the degree of fratricide (e.g., T cells that exhibit severe or mild degrees of fratricide) and/or CAR downregulation depends on the strength of binding between HLA-DR antigen (e.g., in the context of a T cell) and a HLA-DR CAR (e.g., a MVR CAR). The present disclosure demonstrates that fratricide is reduced to a tolerable level when HLA-DR CAR antigen affinity is low. Furthermore, the present disclosure describes a sensitivity tuning mechanism characterized by sustained CAR downregulation that endows HLA-DR CAR T cells (e.g., MVR CAR T cells) with target-cell selectivity based on antigen level and/or affinity.

Thus, the present disclosure provides the insight that a CAR that includes an HLA-DR antigen binding domain (HLA-DR CAR) can be selected, engineered and/or optimized based on the binding characteristics of the HLA-DR binding domain to a T cell from a subject. The present disclosure encompasses a recognition that a HLA-DR CAR that binds to a cell (e.g., a T cell) from a subject with low affinity can provide effective therapy for treating certain diseases and/or disorders (e.g., cancer). Thus the present disclosure provides engineered T cells that include particular HLA-DR CAR polypeptides and/or nucleic acids encoding the same, and moreover demonstrate that these T cells have surprisingly beneficial activity in vitro and in vivo.

### HLA-DR

HLA-DR, (Human Leukocyte Antigen—antigen D Related) is a classic major histocompatibility complex II molecule. (Shackelford, D. A. et al., (1982) Immunol. Rev. 66: 133-187). HLA-DR and its ligand, a peptide of 9 amino acids in length or longer, constitutes a ligand for the TCR. HLA-DR molecules are upregulated in response to signaling. In the instance of an infection, the peptide (such as the staphylococcal enterotoxin I peptide) is bound into a DR molecule and presented to a few of a great many T-cell receptors found on T-helper cells. These cells then bind to antigens on the surface of B-cells stimulating B-cell proliferation.

The primary function of HLA-DR is to present peptide antigens, potentially foreign in origin, to the immune system for the purpose of eliciting or suppressing T-(helper)-cell responses that eventually lead to the production of antibodies against the same peptide antigen. HLA-DR is an αβ heterodimer, cell surface receptor, each subunit of which contains two extracellular domains, a membrane-spanning domain and a cytoplasmic tail. Both α and β chains are anchored in the membrane. The N-terminal domain of the mature protein forms an alpha-helix that constitutes the exposed part of the binding groove, the C-terminal cytoplasmic region interact with the other chain forming a beta-sheet under the binding groove spanning to the cell membrane. The majority of the peptide contact positions are in the first 80 residues of each chain.

HLA-DR has restricted expressed on antigen presenting cells, e.g., DCs, macrophasges, monocytes, and B cells. Increased abundance of DR ‘antigen’ on the cell surface is often in response to stimulation, and, therefore, DR is also a marker for immune stimulation. Due to the high expression level of HLA-DR in B cell malignancies and the limited expression spectrum on normal cells, antibodies against HLA-DR have been developed and tested for B cell malignancies in preclinical and clinical studies. (Nagy, Z. A., et al. (2002) Nat. Med. 8: 801-807; DeNardo, G. L., et al. (2005) Clin. Cancer Res. 11: 7075s-7079s; Ivanov, A., et al. (2009) J. Clin. Invest. 119: 2143-2159; Lin, T. S., et al. (2009) Leuk. Lymphoma 50: 1958-1963). In a phase I/II trial, although the toxicity was not serious, further study was discontinued due to limited efficacy (Lin, T. S., et al. (2009) Leuk. Lymphoma 50: 1958-1963). The present disclosure encompasses a recognition that, given the potential of CAR T cells to augment therapeutic efficacy of monoclonal antibodies by integrating the antigen specificity into the massive T cell response, HLA-DR-redirected CAR T cells can be a useful therapeutics for B cell malignancies.

### HLA-DR CAR

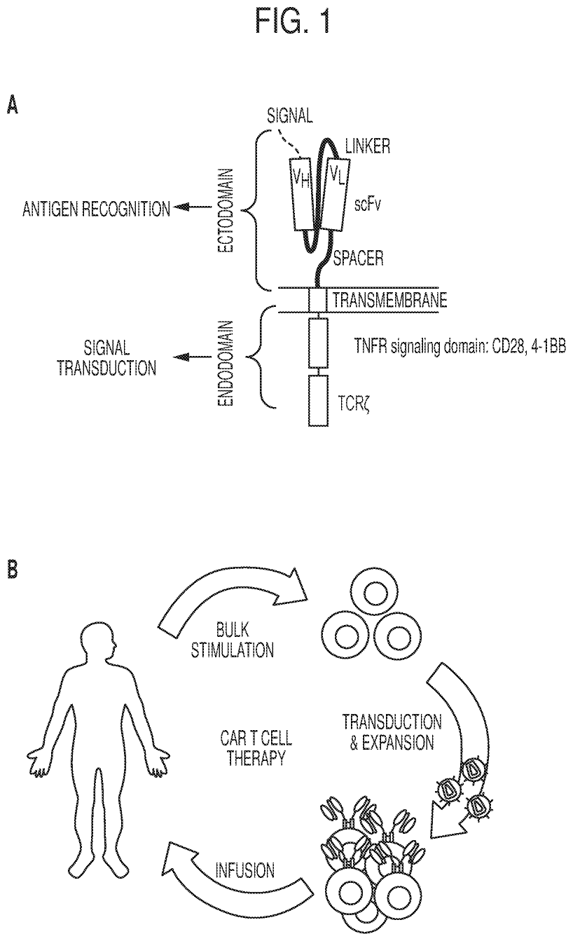

The present disclosure provides, at least in part, HLA-DR CAR polypeptides. The term “chimeric antigen receptor (CAR)” used herein refers to a receptor not present in nature and is capable of providing an immune effector cell with a specificity to a particular antigen. Normally, the CAR refers to a receptor used for delivering the specificity of a monoclonal antibody agent to a T cell. Generally, a CAR comprises an extracellular domain (Ectodomain), a transmembrane domain, and an intracellular domain (Ectodomain). A schematic of an exemplary CAR construct in accordance with the present disclosure is shown in FIG. 1A. In some embodiments, a extracellular domain of a CAR comprises an antigen binding domain. In some embodiments, an antigen binding domain is or comprises an antibody agent. In some embodiments, an antigen binding domain is or comprises an antibody agent that specifically binds to HLA-DR.

Recently, our group developed an antibody agent, MVR, which recognize polymorphic region of HLA-DR (described in U.S. Patent Application Publication No. US 2016-0257762, which is herein incorporated by reference in its entirety). In some embodiments, a HLA-DR CAR comprises a HLA-DR antibody agent. In some embodiments, a HLA-DR CAR comprises a MVR antibody agent.

In some embodiments, a HLA-DR antibody agent is a MVR antibody agent. In some embodiments, the present disclosure provides a chimeric antigen receptor (CAR) protein, including: i) an antibody agent including a heavy chain variable region having one, two or three heavy chain CDRs that are at least 80%, 85%, 90% or 95% identical to a heavy chain CDR sequence as set forth in any one of SEQ ID NOs:2-4; ii) a transmembrane domain; and iii) an intracellular signaling domain, which leads to T cell activation when an antigen binds to the antibody agent.

In some embodiments, a HLA-DR antibody agent is a MVR antibody agent. In some embodiments, the present disclosure provides a chimeric antigen receptor (CAR) protein, including: i) an antibody agent including a light chain variable region having one, two or three light chain CDRs that are at least 80%, 85%, 90% or 95% identical to a light chain CDR sequence as set forth in any one of SEQ ID NOs:6-8; ii) a transmembrane domain; and iii) an intracellular signaling domain, which leads to T cell activation when an antigen binds to the antibody agent.

In some embodiments, a HLA-DR antibody agent is a MVR antibody agent. In some embodiments, the present disclosure provides a chimeric antigen receptor (CAR) protein, including: i) an antibody agent including a heavy chain variable region having one, two or three heavy chain CDRs that are at least 80%, 85%, 90% or 95% identical to a heavy chain CDR sequence as set forth in any one of SEQ ID NOs:2-4; and a light chain variable region having one, two or three light chain CDRs that are at least 80%, 85%, 90% or 95% identical to a light chain CDR sequence as set forth in any one of SEQ ID NOs:6-8; ii) a transmembrane domain; and iii) an intracellular signaling domain, which leads to T cell activation when an antigen binds to the antibody agent.

In some embodiments, a HLA-DR antibody agent is a MVR antibody agent. In some embodiments, the present disclosure provides a chimeric antigen receptor (CAR) protein, including: i) an antibody agent including a heavy chain variable region having one, two or three heavy chain CDRs comprising or consisting of a heavy chain CDR sequence as set forth in any one of SEQ ID NOs:2-4; and a light chain variable region having one, two or three light chain CDRs comprising or consisting of a light chain CDR sequence as set forth in any one of SEQ ID NOs:6-8; ii) a transmembrane domain; and iii) an intracellular signaling domain, which leads to cell activation when an antigen binds to the antibody agent.

In some embodiments, a HLA-DR antibody agent is a MVR antibody agent. In some embodiments, the present disclosure provides a chimeric antigen receptor (CAR) protein, including: i) an antibody agent comprising a heavy chain variable region having a heavy chain CDR1 as set forth in SEQ ID NO:2; a heavy chain CDR2 as set forth in SEQ ID NO:3; and a heavy chain CDR3 as set forth in SEQ ID NO:4; and a light chain variable region having a light chain CDR1 as set forth in SEQ ID NO: 6; a light chain CDR2 as set forth in SEQ ID NO:7; and a light chain CDR3 as set forth in SEQ ID NO:8; ii) a transmembrane domain; and iii) an intracellular signaling domain, which leads to T cell activation when an antigen binds to the antibody agent.

| | SEQUENCE | SEQ ID NO: |
|---|---|---|
| heavy chain CDR 1 | RYSVH | 2 |
| heavy chain CDR 2 | MIWGGGSTDYNSALKS | 3 |
| heavy chain CDR 3 | CARNEGDTTAGTWFAYW | 4 |
| light chain CDR 1 | KASDHINN WLA | 6 |
| light chain CDR 2 | GATSLET | 7 |
| light chain CDR 3 | QQYWSTPFT | 8 |

In some embodiments, the present disclosure provides a chimeric antigen receptor (CAR) protein, including: i) an antibody agent including a heavy chain variable region with an amino acid sequence that is at least 80%, 85%, 90%, 91%, 92%, 93%, 94%, 95%, 96%, 97%, 98%, or 99% identical to a sequence as set forth in SEQ ID NO: 1 and a light chain variable region with an amino acid sequence that is at least 80%, 85%, 90%, 91%, 92%, 93%, 94%, 95%, 96%, 97%, 98%, or 99% identical to a sequence as set forth in SEQ ID NO: 5; ii) a transmembrane domain; and iii) an intracellular signaling domain, which leads to T cell activation when an antigen binds to the antibody agent.

In some embodiments, the present disclosure provides a chimeric antigen receptor (CAR) protein, including: i) an antibody agent including a heavy chain variable region with an amino acid sequence that comprises or consists of a sequence as set forth in SEQ ID NO: 1 and a light chain variable region with an amino acid sequence that comprises or consists of a sequence as set forth in SEQ ID NO: 5; ii) a transmembrane domain; and iii) an intracellular signaling domain, which leads to T cell activation when an antigen binds to the antibody agent.

SEQ ID NO: 1
MVR heavy chain variable region
QVQLKESGPGLVAPSQSLSITCTVSGFSLSRYSVHWVRQPPGKGLEWLGM
IWGGGSTDYNSALKSRLSISKDNSKSQVFLKMNSLQTDDTAMYYCARNEG
DTTAGTWFAYWGQGTLVTVSA
SEQ ID NO: 5
MVR light chain variable region
DIQMTQSSSYLSVSLGGRVTITCKASDHINNWLAWYQQKPGNAPRLLISG
ATSLETGVPSRFSGSGSGKDYTLSITSLQTEDVATYYCQQYWSTPFTFGS
GTKLEIK

In some embodiments, a CAR includes a transmembrane domain of a CAR is connected (e.g., fused, covalently linked) to an extracellular domain. A transmembrane domain of a CAR may be derived from a natural or synthetic transmembrane domain. When it is derived from the naturally present one, it may be one derived from a membrane-bound or transmembrane protein, and may be one derived from α,β, or ξ chain of a T cell receptor, transmembrane regions of various proteins such as CD28, CD3 epsilon, CD45, CD4, CDS, CDS, CD9, CD16, CD22, CD33, CD37, CD64, CD80, CD86, CD134, CD137, CD 154, and CD8. A sequence of a transmembrane domain may be obtained from published references in the art which disclose transmembrane domain of a transmembrane protein, but is not limited thereto.

Additionally, when a transmembrane domain is a synthetic one, it may mainly include hydrophobic amino acid residues such as leucine and valine, for example, it may be present in a transmembrane domain wherein a triplet of phenylalanine, tryptophane, and valine are synthesized, but is not limited thereto. Sequence information on a transmembrane domain may be obtained from published references in the art, but is not limited thereto. In an exemplary embodiment of the present disclosure, CD8-hinge region was used as a transmembrane domain.

In some embodiments, an intracellular domain in a CAR of the present disclosure is part of the CAR domain, and is in a form connected to a transmembrane domain. An intracellular domain of the present disclosure may include an intracellular signaling domain, which is characterized in that it leads to T cell activation when an antigen binds to an antigen-binding region of the CAR, and preferably, T cell proliferation.

An intracellular signaling domain is not particularly limited in its type insofar as it is a signaling part that can lead to T cell activation when an antigen binds to the antigen-binding region present extracellularly. In some embodiments, an intracellular signaling domain includes, for example, an immunoreceptor tyrosine-based activation motif (ITAM), wherein the ITAM includes ones derived from CD3 zeta (ξ), FcR gamma, FcR beta, CD3 gamma, CD3 delta, CD3 epsilon, CDS, CD22, CD79a, CD79b, CD66d or FcεRIγ, but is not limited thereto.

Additionally, an intracellular domain of the CAR of the present disclosure preferably includes a co-stimulatory domain along with the intracellular signaling domain, but is not limited thereto.

A co-stimulatory domain plays a role, at least in part, in delivering a signal to T cells, in addition to the signal by the intracellular signaling domain being included in the CAR of the present invention, and refers to an intracellular part of the CAR, including the intracellular domain of a co-stimulatory molecule.

A co-stimulatory molecule, being a cell surface molecule, refers to a molecule necessary for a sufficient response of a lymphocyte to an antigen. In some embodiments, a co-stimulatory molecule can be or comprise, for example, CD27, CD28, 4-1BB, OX40, CD30, CD40, PD-1, ICOS, lymphocyte function-associated antigen-1 (LFA-1), CD2, CD7, LIGHT, NKG2C, or B7-H3, but is not limited thereto. In some embodiments, a co-stimulatory domain may be an intracellular part of a molecule selected from the group consisting of CD27, CD28, 4-1BB, OX40, CD30, CD40, PD-1, ICOS, lymphocyte function-associated antigen-1 (LFA-1), CD2, CD7, LIGHT, NKG2C, or B7-H3 and a combination thereof.

Additionally, in some embodiments, a short oligopeptide or polypeptide linker may connect the intracellular domain of a CAR and the transmembrane domain, and the linker may not be particularly limited with respect to its length insofar as it is a linker that can induce T cell activation through the intracellular domain when an antigen binds to the antigen binding domain present in an extracellular position, for example, GGGGSGGGGSGGGGS (SEQ ID NO:10) called (GLY₄SER)₃.

In some embodiments, V_{H} and V_{L} parts of an anti-MVR antibody agent can be connect by a (GLY₄SER)₃ linker to construct a MVR scFv. In some embodiments, a CAR comprises a MVR scFv. In some embodiments, a MVR CAR includes a CD8-hinge as a transmembrane domain. In some embodiments, a MVR CAR includes a 4-1BB intracellular domain. In some embodiments, a MVR CAR includes an intracellular domain of the CD3ξ chain. In some embodiments, a MVR CAR includes a MVR scFv, a CD8-hinge, a 4-1BB intracellular domain and an intracellular domain of the CD3 chain.

In some embodiments, the present disclosure provides a chimeric antigen receptor (CAR) protein that comprises a sequence that is at least 60%, 65%, 70%, 75%, 80%, 85%, 90%, 91%, 92%, 93%, 94%, 95%, 96%, 97%, 98%, or 99% identical to a sequence as set forth in SEQ ID NO: 9. In some embodiments, the present disclosure provides a chimeric antigen receptor (CAR) protein that comprises a sequence as set forth in SEQ ID NO: 9.

SEQ ID NO: 9
Exemplary HLA-DR (MVR) CAR
MALPVTALLLPLALLLHAARPDIQMTQSSSYLSVSLGGRVTITCKASDHI
NNWLAWYQQKPGNAPRLLISGATSLETGVPSRFSGSGSGKDYTLSITSLQ
TEDVATYYCQQYWSTPFTFGSGTKLEIKGGGGSGGGGSGGGGSQVQLKES
GPGLVAPSQSLSITSTVSGFSLSRYSVHWVRQPPGKGLEWLGMIWGGGST
DYNSALKSRLSISKDNSKSQVFLKMNSLQTDDTAMYYCARNEGDTTAGTW
FAYWGQGTLVTVSSTTTPAPRPPTPAPTIASQPLSLRPEACRPAAGGAVH
TRGLDFACDIYIWAPLAGTCGVLLLSLVITLYCKRGRKKLLYIFKQPFMR
PVQTTQEEDGCSCRFPEEEEGGCELRVKFSRSADAPAYKQGQNQLYNELN
LGRREEYDVLDKRRGRDPEMGGKPRRKNPQEGLYNELQKDKMAEAYSEIG
MKGERRRGKGHDGLYQGLSTATKDTYDALHMQALPPR

As described in the Examples below, an exemplary HLA-DR antibody agent, MVR, recognizes a variable epitope of HLA-DR. Due, at least in part to epitope variability among subjects, B cells from different subjects (i.e., donors) exhibit different binding affinity. For example, some subjects (i.e., donors) with distinct HLA-DRB1 alleles exhibited extremely low binding by an exemplary MVR-scFv. Using the PBMCs isolated from a subject characterized as a MVR low binder (i.e., DR^{weak}), MVR-engineered CAR T cells (MVR-CAR T cells) with tolerable fratricide were successfully generated. In some embodiments, a HLA-DR CAR T cell is engineered from a subject characterized as a low binder (i.e., expressing an HLA-DR variant that binds with low affinity and/or avidity to an HLA-DR CAR). In some embodiments, a HLA-DR CAR is engineered to have low affinity and/or avidity to T cells from a subject. In some embodiments, a HLA-DR CAR is engineered to have low affinity and/or avidity for a HLA-DR from a subject. In some embodiments, a HLA-DR CAR is selected for expression in a T cell if the affinity and/or avidity of an HLA-DR antigen binding domain to a T cell from a subject is less than a threshold value.

In some embodiments, such a HLA-DR CAR T cell can specifically induce cytotoxicity against a malignant cell. As demonstrated below, such a HLA-DR CAR T cell can specifically induce cytotoxicity against a Epstein-Barr virus-induced lymphoblastoid cell line (EBV-LCL) while sparing normal B cells. The HLA-DR up-regulation in EBV-LCLs and a consequent increase of granule transfer rate was involved in this mechanism. The examples below demonstrate the proof-of-concept of malignancy-specific killing of HLA-DR-redirected MVR-CAR T cells in B cell lymphoma, and highlights the therapeutic benefits of HLA-DR CAR T cells produced via methods of the present disclosure.

### Nucleic Acids

The disclosure provides polynucleotides comprising a nucleotide sequence encoding HLA-DR CARs of the present disclosure. HLA-DR CARs as described herein may be produced from nucleic acid molecules using molecular biological methods known to the art. Nucleic acids of the present disclosure include, for example, DNA and/or RNA.

In some embodiments, nucleic acid constructs include regions that encode a HLA-DR CAR. A HLA-DR CAR may be identified and/or selected for a desired binding and/or functional properties, and variable regions of said antibody agent isolated, amplified, cloned and/or sequenced. Modifications may be made to the variable region nucleotide sequences, including additions of nucleotide sequences encoding amino acids and/or carrying restriction sites, and/or substitutions of nucleotide sequences encoding amino acids. In some embodiments, a nucleic acid sequence may or may not include an intron sequence.

Nucleic acid constructs of the present disclosure may be inserted into an expression vector or viral vector by methods known to the art, and nucleic acid molecules may be operably linked to an expression control sequence. A vector comprising any of the above-described nucleic acid molecules, or fragments thereof, is further provided by the present disclosure. Any of the above nucleic acid molecules, or fragments thereof, can be cloned into any suitable vector and can be used to transform or transfect any suitable host. The selection of vectors and methods to construct them are commonly known to persons of ordinary skill in the art and are described in general technical references (see, in general, “Recombinant DNA Part D,” Methods in Enzymology, Vol. 153, Wu and Grossman, eds., Academic Press (1987)).

In some embodiments, conventionally used techniques, such as, fore example, electrophoresis, calcium phosphate precipitation, DEAE-dextran transfection, lipofection, etc. may be used to introduce a foreign nucleic acid (DNA or RNA) into a prokaryotic or eukaryotic host cell. Desirably, a vector may include regulatory sequences, such as transcription and translation initiation and termination codons, which are specific to the type of host (e.g., bacterium, fungus, plant or animal) into which the vector is to be introduced, as appropriate and taking into consideration whether the vector is DNA or RNA. In some embodiments, a vector comprises regulatory sequences that are specific to the genus of the host. Preferably, a vector comprises regulatory sequences that are specific to the species of the host.

In addition to the replication system and the inserted nucleic acid, a nucleic acid construct can include one or more marker genes, which allow for selection of transformed or transfected hosts. Marker genes include biocide resistance, e.g., resistance to antibiotics, heavy metals, etc., complementation in an auxotrophic host to provide prototrophy, and the like.

Suitable vectors include those designed for propagation and expansion or for expression or both. For example, a cloning vector is selected from the group consisting of the pUC series, the pBluescript series (Stratagene, LaJolla, Calif.), the pET series (Novagen, Madison, Wis.), the pGEX series (Pharmacia Biotech, Uppsala, Sweden), and the pEX series (Clontech, Palo Alto, Calif.). Bacteriophage vectors, such as λGT10, λGT11, λZapII (Stratagene), λEMBL4, and λNM1149, also can be used. Examples of plant expression vectors include pBI110, pBI101.2, pBI101.3, pBI121 and pBIN19 (Clontech). Examples of animal expression vectors include pEUK-C1, pMAM and pMAMneo (Clontech). The TOPO cloning system (Invitrogen, Carlsbad, Calif.) also can be used in accordance with the manufacturer's recommendations.

An expression vector can comprise a native or nonnative promoter operably linked to an isolated or purified nucleic acid molecule as described above. Selection of promoters, e.g., strong, weak, inducible, tissue-specific and developmental-specific, is within the skill in the art. Similarly, combining of a nucleic acid molecule, or fragment thereof, as described above with a promoter is also within the skill in the art.

Suitable viral vectors include, for example, retroviral vectors, parvovirus-based vectors, e.g., adeno-associated virus (AAV)-based vectors, AAV-adenoviral chimeric vectors, and adenovirus-based vectors, and lentiviral vectors, such as Herpes simplex (HSV)-based vectors. These viral vectors can be prepared using standard recombinant DNA techniques described in, for example, Sambrook et al., Molecular Cloning, a Laboratory Manual, 2d edition, Cold Spring Harbor Press, Cold Spring Harbor, N.Y. (1989); and Ausubel et al., Current Protocols in Molecular Biology, Greene Publishing Associates and John Wiley & Sons, New York, N.Y. (1994).

A retroviral vector is derived from a retrovirus. Retrovirus is an RNA virus capable of infecting a wide variety of host cells. Upon infection, the retroviral genome integrates into the genome of its host cell and is replicated along with host cell DNA, thereby constantly producing viral RNA and any nucleic acid sequence incorporated into the retroviral genome. As such, long-term expression of a therapeutic factor(s) is achievable when using retrovirus. Retroviruses contemplated for use in gene therapy are relatively non-pathogenic, although pathogenic retroviruses exist. When employing pathogenic retroviruses, e.g., human immunodeficiency virus (HIV) or human T-cell lymphotrophic viruses (HTLV), care must be taken in altering the viral genome to eliminate toxicity to the host. A retroviral vector additionally can be manipulated to render the virus replication-deficient. As such, retroviral vectors are considered particularly useful for stable gene transfer in vivo. Lentiviral vectors, such as HIV-based vectors, are exemplary of retroviral vectors used for gene delivery. Unlike other retroviruses, HIV-based vectors are known to incorporate their passenger genes into non-dividing cells and, therefore, can be of use in treating persistent forms of disease.

Additional sequences can be added to such cloning and/or expression sequences to optimize their function in cloning and/or expression, to aid in isolation of the polynucleotide, or to improve the introduction of the polynucleotide into a cell. Use of cloning vectors, expression vectors, adapters, and linkers is well known in the art. (See, e.g., Ausubel, supra; or Sambrook, supra).

In some embodiments, nucleic acids and vectors of the present disclosure may be isolated and/or purified. The present disclosure also provides a composition comprising an above-described isolated or purified nucleic acid molecule, optionally in the form of a vector. Isolated nucleic acids and vectors may be prepared using standard techniques known in the art including, for example, alkali/SDS treatment, CsCl binding, column chromatography, agarose gel electrophoresis and other techniques well known in the art. The composition can comprise other components as described further herein.

In some embodiments, nucleic acid molecules are inserted into a vector that is able to express an HLA-DR CAR when introduced into an appropriate cell. In some embodiments, a cell is a T cell.

Any method(s) known to one skilled in the art for the insertion of DNA fragments into a vector may be used to construct expression vectors encoding a HLA-DR CAR of the present disclosure under control of transcriptional/translational control signals. These methods may include in vitro recombinant DNA and synthetic techniques and in vivo recombination (See, e.g., Ausubel, supra; or Sambrook, supra).

### Production of HLA-DR CAR-T Cells

Yet another object of the present invention is to provide methods for producing T cells comprising a HLA-DR CAR. In some embodiments, a T cell of the present where a CAR is introduced therein is a CD4⁺ T cell (helper T cell, T_{H} cell), a CD8⁺ T cell (cytotoxic T cell, CTL), a memory T cell, a regulatory T cell (Treg cell), an apoptotic T cell, but is not limited thereto. In some embodiments, a T cell of the present where a CAR is introduced therein is a CD8⁺ T cell. In some embodiments, a T cell of the present where a CAR is introduced therein is a CD4⁺ T cell.

In some embodiments, the present disclosure provides methods of producing an autologous engineered T cell of the present disclosure, comprising: (a) obtaining a HLA-DR antigen binding domain, wherein HLA-DR antigen binding domain binds to HLA-DR from a subject with low affinity, and (b) expressing a chimeric antigen receptor (CAR) comprising the HLA-DR antigen binding domain in a T cell obtained from the subject, thereby producing the autologous engineered T cell. In some embodiments, a method of producing an autologous engineered T cell of the present disclosure further comprises culturing the autologous engineered T cell in vitro for at least 8 days, 9 days, 10 days, 11 days, 12 days, 13 days, or 14 days.

In some embodiments, the present disclosure provides methods of preparing an autologous engineered T cell of the present disclosure, comprising: providing or obtaining an analysis of binding of a HLA-DR antigen binding domain to a T cell from a subject; and if the binding is less than a threshold value, engineering a T cell from the subject to express a CAR comprising the HLA-DR antigen binding domain. In some embodiments, a method of producing an autologous engineered T cell of the present disclosure further comprises culturing the autologous engineered T cell in vitro for at least 8 days, 9 days, 10 days, 11 days, 12 days, 13 days, or 14 days.

In some embodiments, the step of culturing in the provided methods produces a population of autologous engineered T cells with reduced surface expression of the CAR relative to a population of the autologous engineered T cells that has been cultured in vitro for 2 days.

In some embodiments, the step of culturing in the provided methods produces a population of autologous engineered T cells with reduced toxicity towards normal B cells relative to a population of the autologous engineered T cells that has been cultured in vitro for 2 days.

In some embodiments, the step of culturing in the provided methods produces a population of autologous engineered T cells that has enhanced selectivity for malignant cells over to non-malignant cells relative to a population of the autologous engineered T cells that has been cultured in vitro for 2 days.

In some embodiments, an autologous engineered T cell in the context of the present disclosure exhibits granual transfer EBV LCLs is at least two times more than the granual transfer of the engineered T cell to normal B cells from the subject.

Any appropriate method for analyzing binding of an antigen binding domain to a T cell or antigen known in the art may be used in the context of the present disclosure. In some embodiments, an analysis of binding of a HLA-DR antigen binding domain to a T cell from a subject can be an assessment of T cell avidity. In some embodiments, avidity of T cells can be assessed on a scale that integrates the expression level of the receptor and receptor-antigen affinity. (See, e.g., Vigano, S. et al. (2012) Clin. Dev. Immunol. 2012: 153863). In some embodiments, T cell avidity can be a measure of a minimum antigen level above which TCR-antigen complexes form clusters that eventually lead to T cell activation.

In some embodiments, an analysis of binding of a HLA-DR antigen binding domain to a T cell from a subject is a direct measurement of binding affinity (e.g., K_{D}). In some embodiments, an analysis of binding of a HLA-DR antigen binding domain to a T cell from a subject is a measure of functional avidity of a HLA-DR antigen binding domain to a T cell. In some embodiments, the functional avidity inversely correlates with the antigen dose that is needed to trigger a T-cell response. In some embodiments, a measure of functional avidity of a HLA-DR antigen binding domain to a T cell includes ex vivo quantification of T cell functions such as, for example, IFN-γ production, cytotoxic activity (ability to lyse target cells), or proliferation. In some embodiments, a measure of functional avidity of a HLA-DR antigen binding domain to a T cell includes determining a concentration of a HLA-DR antigen binding domain needed to induce a half-maximum response (EC₅₀) of T cells.

Any method known in the art for expressing a CAR in T cells can be used in the context of the present disclosure. For example, there are various nucleic acid vectors for expression known in the art, such as, for example, linear polynucleotides, polynucleotides to which an ionic or amphiphilic compound is bound, plasmids, viral vectors, ect, though the present disclosure is not limited thereto. In some embodiments, a vector for expression of a CAR in T cells may be or include an autonomously replicating plasmid or virus or derivative thereof. Viral vectors can include, but are not limited to adenovirus vector, adeno-associated viral vector, retrovirus vector, etc. In some embodiments a lentivirus vector, which is a retroviral vector, can be used. In some embodiments, a vector is a non-plasmid and a non-viral compound, such as, for example, a liposome.

In some embodiments, lymphocytes (e.g., T cells) are cultured at a temperature of at least about 25° C., preferably at least about 30° C., more preferably about 37° C.

The present disclosure encompasses the recognition that HLA-DR CAR T cells, generated by the methods described herein may be therapeutically useful (e.g., for the treatment of cancer). In some embodiments, a HLA-DR CAR T cell is engineered to best suit the HLA-DR variant of a patient in need of treatment.

### Therapeutic Applications

The present disclosure provides methods for HLA-DR CAR T cell therapy. In some embodiments a HLA-DR CAR T cell therapy is an autologous CAR T cell therapy. A generic schematic illustrating overarching steps involved in autologous CAR T cell therapy are depicted in FIG. 1B. These steps include isolation and bulk stimulation of T cells from a subject in need of CAR T cell therapy, transduction and expansion of CAR T cells, and infusion of a composition that comprises or delivers CAR T cells.

In some embodiments, the present disclosure provides methods of producing a therapeutic preparation, comprising: providing or obtaining an analysis of avidity of an engineered T cell comprising a CAR comprising a HLA-DR antigen binding domain for an HLA-DR antigen of a subject, and if the avidity is less than a threshold value, producing a therapeutic preparation comprising the engineered T cell. In some embodiments, an analysis of avidity of an engineered T cell comprising a CAR comprising a HLA-DR antigen binding domain for an HLA-DR antigen of a subject is an analysis of functional avidity. In some embodiments, a measure of functional avidity of a HLA-DR antigen binding domain to a T cell includes ex vivo quantification of T cell functions such as, for example, IFN-γ production, cytotoxic activity (ability to lyse target cells), or proliferation.

In some embodiments, a method for producing a therapeutic preparation comprises: providing or obtaining an analysis of functional avidity of an engineered T cell comprising a CAR comprising a HLA-DR antigen binding domain for an HLA-DR antigen of a subject, and if the functional avidity is less than a threshold value, producing a therapeutic preparation comprising the engineered T cell. In some embodiments, a measure of functional avidity is proliferation of an engineered T cell when cultured for at least 8 days, 10 days, 12 days or 14 days with an appropriate stimulation. In some embodiments, an appropriate stimulation includes exposing the T cell to a CD3-specific antibody and/or a HLA-DR-expressing cell. In some embodiments, a threshold value of functional avidity is at least 15-fold, 20-fold, 25-fold proliferation.

In some embodiments, a method for producing a therapeutic preparation comprises: providing or obtaining an analysis of functional avidity of an engineered T cell comprising a CAR comprising a HLA-DR antigen binding domain for an HLA-DR antigen of a subject, and if the functional avidity is less than a threshold value, producing a therapeutic preparation comprising the engineered T cell, wherein the threshold value is at least 15-fold, 20-fold, 25-fold proliferation of an engineered T cell when cultured for at least 12 days with a CD3-specific antibody and/or a HLA-DR-expressing cell.

In some embodiments, the present disclosure provides methods of treating a subject in need thereof, the method comprising administering to the subject a composition that comprises or delivers a T cell comprising a HLA-DR CAR. In some embodiments a T cell comprising a HLA-DR CAR is an autologous T cell. In some embodiments, a HLA-DR binding domain of an HLA-DR CAR has low affinity for a T cell from a subject that is to be administered a pharmaceutical composition. In some embodiments, the present disclosure provides methods of treating a subject in need thereof, the method comprising administering to the subject a composition that comprises or delivers a T cell comprising a nucleic acid and/or vector encoding a HLA-DR CAR. In some embodiments a T cell comprising a nucleic acid and/or vector encoding a HLA-DR CAR is an autologous T cell. In some embodiments, a HLA-DR binding domain of an HLA-DR CAR has low affinity for a T cell from a subject to be administered a pharmaceutical composition. In some embodiments, a subject has or is at risk for developing cancer.

In some embodiments, the present disclosure provides methods of inducing an immune response in a subject in need thereof, the method comprising administering to the subject a composition that comprises or delivers a T cell comprising a HLA-DR CAR. In some embodiments a T cell comprising a HLA-DR CAR is an autologous T cell. In some embodiments, a HLA-DR binding domain of an HLA-DR CAR has low affinity for a T cell from a subject that is to be administered a pharmaceutical composition. In some embodiments, the present disclosure provides methods of inducing an immune response in a subject in need thereof, the method comprising administering to the subject a composition that comprises or delivers a T cell comprising a nucleic acid and/or vector encoding a HLA-DR CAR. In some embodiments a T cell comprising a nucleic acid and/or vector encoding a HLA-DR CAR is an autologous T cell. In some embodiments, a HLA-DR binding domain of an HLA-DR CAR has low affinity for a T cell from a subject to be administered a pharmaceutical composition. In some embodiments, a subject has or is at risk for developing cancer.

In some embodiments, the present disclosure provides methods of enhancing an immune response in a subject in need thereof, the method comprising administering to the subject a composition that comprises or delivers a T cell comprising a HLA-DR CAR. In some embodiments a T cell comprising a HLA-DR CAR is an autologous T cell. In some embodiments, a HLA-DR binding domain of an HLA-DR CAR has low affinity for a T cell from a subject that is to be administered a pharmaceutical composition. In some embodiments, the present disclosure provides methods of enhancing an immune response in a subject in need thereof, the method comprising administering to the subject a composition that comprises or delivers a T cell comprising a nucleic acid and/or vector encoding a HLA-DR CAR. In some embodiments a T cell comprising a nucleic acid and/or vector encoding a HLA-DR CAR is an autologous T cell. In some embodiments, a HLA-DR binding domain of an HLA-DR CAR has low affinity for a T cell from a subject to be administered a pharmaceutical composition. In some embodiments, a subject has or is at risk for developing cancer.

In some embodiments, a disease suitable for treatment with compostions and methods of the present disclosure is selected from a proliferative disease such as a cancer or malignancy or a precancerous condition. In some embodiments, a disease is associated with expression of HLA-DR. In some embodiments, a disease suitable for treatment with compostions and methods of the present disclosure is a cancer. In some embodiments, a cancer expresses a HLA-DR antigen. In some embodiments, a cancer cell has increased expression of HLA-DR antigen relative to a non-cancer cell from a subject. In some embodiments, a cancer cell has at least 1.5-fold, 2-fold, 3-fold, 4-fold, 5-fold, or 6-fold higher expression of HLA-DR antigen relative to a non-cancer cell from a subject. In some certain embodiments, a cancer suitable for treatment with compositions and methods of the present disclosure has an at least 2-fold higher expression of HLA-DR antigen relative to a normal cell of the same type from a subject.

Cancers suitable for treatment by a method of the present disclosure can include, but are not limited to, bladder cancer, breast cancer, cervical cancer, colon cancer, endometrial cancer, esophageal cancer, fallopian tube cancer, gall bladder cancer, gastrointestinal cancer, head and neck cancer, hematological cancer, laryngeal cancer, liver cancer, lung cancer, lymphoma, melanoma, mesothelioma, ovarian cancer, primary peritoneal cancer, salivary gland cancer, sarcoma, stomach cancer, thyroid cancer, pancreatic cancer, and prostate cancer. In some embodiments, a cancer for treatment by a method of the present disclosure can include may include, but is not limited to, carcinoma, lymphoma (e.g., Hodgkin's and non-Hodgkin's lymphomas), blastoma, sarcoma and leukemia. In some embodiments, cancer may include squamous cell carcinoma, small cell lung cancer, non-small cell lung cancer, lung adenocarcinoma, squamous cell carcinoma of the lung, peritoneal cancer, hepatocellular carcinoma, gastric cancer, pancreatic cancer, glioma, cervical cancer, ovarian cancer, liver cancer, bladder cancer, hepatocellular carcinoma, breast cancer, colon cancer, colorectal cancer, endometrial or uterine carcinoma, salivary carcinoma, kidney cancer, prostate cancer, vulvar cancer, thyroid cancer, liver carcinoma, leukemia and other lymphoproliferative disorders, and various types of head and neck cancer.

In some embodiments, a cancer suitable for treatment by methods of the present disclosure is a hematologic cancer. In some embodiments, a hematologic cancer is a leukemia. In some embodiments, a cancer is selected from the group consisting of one or more acute leukemias including but not limited to B-cell acute lymphoid leukemia (“BALL”), T-cell acute lymphoid leukemia (“TALL”), acute lymphoid leukemia (ALL); one or more chronic leukemias including but not limited to chronic myelogenous leukemia (CIVIL), chronic lymphocytic leukemia (CLL); additional hematologic cancers or hematologic conditions including, but not limited to B cell prolymphocytic leukemia, blastic plasmacytoid dendritic cell neoplasm, Burkitt's lymphoma, diffuse large B cell lymphoma, follicular lymphoma, hairy cell leukemia, small cell- or a large cell-follicular lymphoma, malignant lymphoproliferative conditions, MALT lymphoma, mantle cell lymphoma, Marginal zone lymphoma, multiple myeloma, myelodysplasia and myelodysplastic syndrome, non-Hodgkin's lymphoma, plasmablastic lymphoma, plasmacytoid dendritic cell neoplasm, Waldenstrom macroglobulinemia, and “preleukemia” which are a diverse collection of hematological conditions united by ineffective production (or dysplasia) of myeloid blood cells, and to disease associated with HLA-DR expression include, but not limited to atypical and/or non-classical cancers, malignancies, precancerous conditions or proliferative diseases expressing HLA-DR; and any combination thereof.

In some embodiments, a cancer for treatment by methods of the present disclosure is a B cell lymphoma (i.e., a malignant lymphoma of B cell origin). B cell lymphomas include Hodgkin's lymphoma and non-Hodgkin's lymphoma, diffuse large B cell lymphoma (DLBCL), follicular lymphoma, mucosa-associated lymphatic tissue lymphoma (MALT), chronic lymphocytic leukemia, mantle cell lymphoma (MCL), burkitt lymphoma, mediastinal large B cell lymphoma, waldenstrom macroglobulinemia, nodal marginal zone B cell lymphoma (NMZL), splenic marginal zone lymphoma (SMZL), intravascular large B-cell lymphoma, primary effusion lymphoma, lymphomatoid granulomatosis, and AIDS-related lymphoma, but is not particularly limited thereto as long as it is lymphoma of B cell origin.

A composition including a composition that comprises or delivers a T cell comprising a HLA-DR CAR of the present disclosure may be administered at a pharmaceutically effective amount to treat cancer cells or metastasis thereof, or inhibit the growth of cancer. For use in therapeutic methods, T cell comprising a HLA-DR CAR of the present disclosure would be formulated, dosed, and administered in a fashion consistent with good medical practice. Factors for consideration in this context include the particular disorder being treated, the particular mammal being treated, the clinical condition of the individual patient, the age of the patient, the weight of the patient, the cause of the disorder, the site of delivery of the agent, the method of administration, the scheduling of administration, and other factors known to medical practitioners.

In some embodiments, T cells for using in a therapeutic method are autologous (the donor and the recipient are the same). In some embodiments, T cells for using in a therapeutic method are syngeneic (the donor and the recipients are different but are identical twins). In some embodiments, T cells for using in a therapeutic method are allogenic (from the same species but different donor) as the recipient subject.

In some embodiments, a treatment-effective amount of cells in the composition is In some embodiments, a composition includes at least 10⁶, at least 10⁷, at least 10⁸, at least 10⁹, at least 10¹⁰ cells, or more than 10¹⁰ T cells comprising a HLA-DR CAR. The number of cells will depend upon the ultimate use for which the composition is intended as will the type of cells included therein. For example, in some embodiments, a population of T cells comprising a HLA-DR CAR will contain greater than 10%, greater than 15%, greater than 20%, greater than 25%, greater than 30%, or greater than 35% of such cells. In some embodiments, a population of T cells comprising a HLA-DR CAR will contain 10% to 50%, 15% to 45%, 20% to 40%, 25% to 35%, or 20% to 30% of such T cells. For uses provided herein, a population of T cells for administration are generally in a volume of a liter or less. In some embodiments, T cells for administration are in a volume of less than 500 ml, less than 250 ml, or 100 ml or less. In some embodiments, a density of the desired T cells is typically greater than 10⁶ cells/ml and generally is greater than 10⁷ cells/ml, generally 10⁸ cells/ml or greater. A clinically relevant number of immune cells can be apportioned into multiple infusions that cumulatively equal or exceed 10⁷ cells, 10⁸ cells, 10⁹ cells, 10¹⁰ cells. 10¹¹ cells, or 10¹² cells.

In some embodiments, a composition may be administered to a patient parenterally. In some embodiments, a composition that comprises or delivers a T cell comprising a HLA-DR CAR may be parenterally administered to a patient in one or multiple administrations. In some embodiments, a composition that comprises or delivers a T cell comprising a HLA-DR CAR may be parenterally administered to a patient once every day, once every 2 to 7 days, every week, once every two weeks, once every month, once every three months, or once every 6 months.

### Compositions

In some embodiments, the present disclosure provides pharmaceutical compositions that include a T cell comprising a HLA-DR CAR and a pharmaceutically acceptable carrier. In some embodiments a T cell comprising a HLA-DR CAR is an autologous T cell. In some embodiments, a HLA-DR binding domain of an HLA-DR CAR has low affinity for a T cell from a subject that is to be administered a pharmaceutical composition. In some embodiments, the present disclosure provides pharmaceutical compositions that include a T cell comprising a nucleic acid and/or vector encoding a HLA-DR CAR and a pharmaceutically acceptable carrier. In some embodiments a T cell comprising a nucleic acid and/or vector encoding a HLA-DR CAR is an autologous T cell. In some embodiments, a HLA-DR binding domain of an HLA-DR CAR has low affinity for a T cell from a subject to be administered a pharmaceutical composition.

In some embodiments, the present disclosure provides pharmaceutical compositions that include an engineered T cell comprising a HLA-DR CAR and a pharmaceutically acceptable carrier, wherein the engineered T cell has low functional avidity for a T cell from a subject that is to be administered a pharmaceutical composition. In some embodiments, the functional avidity is below a threshold level. In some embodiments, functional avidity of an engineered T cell to a T cell of a subject is assessed using an ex vivo quantification of T cell functions such as, for example, IFN-γ production, cytotoxic activity (ability to lyse target cells), or proliferation. In some embodiments, a measure of functional avidity is proliferation of an engineered T cell when cultured for at least 8 days, 10 days, 12 days or 14 days with an appropriate stimulation. In some embodiments, an appropriate stimulation includes exposing the T cell to a CD3-specific antibody and/or a HLA-DR-expressing cell. In some embodiments, a threshold value of functional avidity is at least 15-fold, 20-fold, 25-fold proliferation.

Compositions of the present disclosure include pharmaceutical compositions that include a T cell comprising a HLA-DR CAR and/or a nucleic acid encoding a HLA-DR CAR obtained by a method disclosed herein. In some embodiments, a pharmaceutical composition can include a buffer, a diluent, an excipient, or any combination thereof. In some embodiments, a composition, if desired, can also contain one or more additional therapeutically active substances.

In some embodiments, a T cell comprising a HLA-DR CAR and/or a nucleic acid encoding a HLA-DR CAR of the present disclosure are suitable for administration to a mammal (e.g., a human). Although the descriptions of pharmaceutical compositions provided herein are principally directed to pharmaceutical compositions that are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation.

In some embodiments, T cells of the present disclosure are formulated by first harvesting them from their culture medium, and then washing and concentrating the cells in a medium and container system suitable for administration (a “pharmaceutically acceptable” carrier) in a treatment-effective amount. Suitable infusion medium can be any isotonic medium formulation, typically normal saline, Normosol R (Abbott) or Plasma-Lyte A (Baxter), but also 5% dextrose in water or Ringer's lactate can be utilized. The infusion medium can be supplemented with human serum albumin.

In some embodiments, compositions are formulated for parenteral administration. For example, a pharmaceutical composition provided herein may be provided in a sterile injectable form (e.g., a form that is suitable for subcutaneous injection or intravenous infusion). For example, in some embodiments, a pharmaceutical compositions is provided in a liquid dosage form that is suitable for injection. In some embodiments, a pharmaceutical composition is provided as powders (e.g., lyophilized and/or sterilized), optionally under vacuum, which can be reconstituted with an aqueous diluent (e.g., water, buffer, salt solution, etc.) prior to injection. In some embodiments, a pharmaceutical composition is diluted and/or reconstituted in water, sodium chloride solution, sodium acetate solution, benzyl alcohol solution, phosphate buffered saline, etc. In some embodiments, a powder should be mixed gently with the aqueous diluent (e.g., not shaken).

In some embodiments, a T cell comprising a HLA-DR CAR and/or a nucleic acid encoding a HLA-DR CAR of the present disclosure is formulated with a pharmaceutically acceptable parenteral vehicle. Examples of such vehicles are water, saline, Ringer's solution, dextrose solution, and 1-10% human serum albumin. Liposomes and nonaqueous vehicles such as fixed oils can also be used. A vehicle or lyophilized powder can contain additives that maintain isotonicity (e.g., sodium chloride, mannitol) and chemical stability (e.g., buffers and preservatives). In some embodiments, a formulation is sterilized by known or suitable techniques.

Formulations of the pharmaceutical compositions described herein may be prepared by any method known or hereafter developed in the art of pharmacology. In general, such preparatory methods include the step of bringing the active ingredient into association with a diluent or another excipient and/or one or more other accessory ingredients, and then, if necessary and/or desirable, packaging the product into a desired single- or multi-dose unit.

In some embodiments, a pharmaceutical composition including a T cell comprising a HLA-DR CAR and/or a nucleic acid encoding a HLA-DR CAR of the present disclosure can be included in a container for storage or administration, for example, an vial, a syringe (e.g., an IV syringe), or a bag (e.g., an IV bag). A pharmaceutical composition in accordance with the present disclosure may be prepared, packaged, and/or sold in bulk, as a single unit dose, and/or as a plurality of single unit doses. As used herein, a “unit dose” is discrete amount of the pharmaceutical composition comprising a predetermined amount of the active ingredient. The amount of the active ingredient is generally equal to the dosage of the active ingredient that would be administered to a subject and/or a convenient fraction of such a dosage such as, for example, one-half or one-third of such a dosage.

Relative amounts of T cells comprising a HLA-DR CAR and/or a nucleic acid encoding a HLA-DR CAR, pharmaceutically acceptable excipient(s), and/or any additional ingredients in a pharmaceutical composition in accordance with the disclosure will vary, depending upon the identity, size, and/or condition of the subject treated and further depending upon the route by which the composition is to be administered. By way of example, a composition may comprise a population of T cells comprising a HLA-DR CAR and/or a nucleic acid encoding a HLA-DR CAR at least 10⁶, at least 10′, at least 10⁸, at least 10⁹, at least 10¹⁰ cells, or more than 10¹⁰ T cells comprising a HLA-DR CAR. The number of cells will depend upon the ultimate use for which the composition is intended as will the type of cells included therein. For example, in some embodiments, a population of T cells comprising a HLA-DR CAR will contain greater than 10%, greater than 15%, greater than 20%, greater than 25%, greater than 30%, or greater than 35% of such cells. In some embodiments, a population of T cells comprising a HLA-DR CAR will contain 10% to 50%, 15% to 45%, 20% to 40%, 25% to 35%, or 20% to 30% of such T cells. For uses provided herein, a population of T cells for administration are generally in a volume of a liter or less. In some embodiments, T cells for administration are in a volume of less than 500 ml, less than 250 ml, or 100 ml or less. In some embodiments, a density of the desired T cells is typically greater than 10⁶ cells/ml and generally is greater than 10⁷ cells/ml, generally 10⁸ cells/ml or greater. A clinically relevant number of immune cells can be apportioned into multiple infusions that cumulatively equal or exceed 10⁷ cells, 10⁸ cells, 10⁹ cells, 10¹⁰ cells. 10¹¹ cells, or 10¹² cells

In some embodiments, a composition comprises or delivers T cells comprising a HLA-DR CAR in an amount within a range bounded by a lower limit and an upper limit, the upper limit being larger than the lower limit. In some embodiments, the lower limit may be about 10⁶ cells, 10⁷ cells, 10⁸ cells, 10⁹ cells, 10¹⁰ cells. 10¹¹ cells, or 10¹² cells. In some embodiments, the upper limit may be about 10⁷ cells, 10⁸ cells, 10⁹ cells, 10¹⁰ cells. 10¹¹ cells, 10¹² cells, 10¹³ cells, or 10¹⁴ cells.

A pharmaceutical composition may additionally comprise a pharmaceutically acceptable excipient, which, as used herein, includes any and all solvents, dispersion media, diluents, or other liquid vehicles, dispersion or suspension aids, surface active agents, isotonic agents, thickening or emulsifying agents, preservatives, solid binders, lubricants and the like, as suited to the particular dosage form desired. Remington's The Science and Practice of Pharmacy, 21st Edition, A. R. Gennaro (Lippincott, Williams & Wilkins, Baltimore, Md., 2006) discloses various excipients used in formulating pharmaceutical compositions and known techniques for the preparation thereof. Except insofar as any conventional excipient medium is incompatible with a substance or its derivatives, such as by producing any undesirable biological effect or otherwise interacting in a deleterious manner with any other component(s) of the pharmaceutical composition, its use is contemplated to be within the scope of this disclosure.

In some embodiments, a pharmaceutically acceptable excipient is at least 95%, at least 96%, at least 97%, at least 98%, at least 99%, or 100% pure. In some embodiments, an excipient is approved for use in humans and for veterinary use. In some embodiments, an excipient is approved by the United States Food and Drug Administration. In some embodiments, an excipient is pharmaceutical grade. In some embodiments, an excipient meets the standards of the United States Pharmacopoeia (USP), the European Pharmacopoeia (EP), the British Pharmacopoeia, and/or the International Pharmacopoeia.

Pharmaceutically acceptable excipients used in the manufacture of pharmaceutical compositions include, but are not limited to, inert diluents, dispersing and/or granulating agents, surface active agents and/or emulsifiers, disintegrating agents, binding agents, preservatives, buffering agents, lubricating agents, and/or oils. Such excipients may optionally be included in pharmaceutical formulations. Excipients such as cocoa butter and suppository waxes, coloring agents, coating agents, sweetening, flavoring, and/or perfuming agents can be present in the composition, according to the judgment of the formulator.

In some embodiments, a provided pharmaceutical composition comprises one or more pharmaceutically acceptable excipients (e.g., preservative, inert diluent, dispersing agent, surface active agent and/or emulsifier, buffering agent, etc.). In some embodiments, a pharmaceutical composition comprises one or more preservatives. In some embodiments, pharmaceutical compositions comprise no preservative.

In some embodiments, a composition including a population of T cells comprising a HLA-DR CAR and/or a nucleic acid encoding a HLA-DR CAR of the present disclosure is stably formulated. In some embodiments, a stable formulation of a population of T cells comprising a HLA-DR CAR and/or a nucleic acid encoding a HLA-DR CAR of the present disclosure may comprise a phosphate buffer with saline or a chosen salt, as well as preserved solutions and formulations containing a preservative as well as multi-use preserved formulations suitable for pharmaceutical or veterinary use. Preserved formulations contain at least one known preservative or optionally selected from the group consisting of at least one phenol, m-cresol, p-cresol, o-cresol, chlorocresol, benzyl alcohol, phenylmercuric nitrite, phenoxyethanol, formaldehyde, chlorobutanol, magnesium chloride (e.g., hexahydrate), alkylparaben (methyl, ethyl, propyl, butyl and the like), benzalkonium chloride, benzethonium chloride, sodium dehydroacetate and thimerosal, or mixtures thereof in an aqueous diluent. Any suitable concentration or mixture can be used as known in the art, such as 0.001-5%, or any range or value therein, such as, but not limited to 0.001, 0.003, 0.005, 0.009, 0.01, 0.02, 0.03, 0.05, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.3, 4.5, 4.6, 4.7, 4.8, 4.9, or any range or value therein. Non-limiting examples include, no preservative, 0.1-2% m-cresol (e.g., 0.2, 0.3. 0.4, 0.5, 0.9, 1.0%), 0.1-3% benzyl alcohol (e.g., 0.5, 0.9, 1.1, 1.5, 1.9, 2.0, 2.5%), 0.001-0.5% thimerosal (e.g., 0.005, 0.01), 0.001-2.0% phenol (e.g., 0.05, 0.25, 0.28, 0.5, 0.9, 1.0%), 0.0005-1.0% alkylparaben(s) (e.g., 0.00075, 0.0009, 0.001, 0.002, 0.005, 0.0075, 0.009, 0.01, 0.02, 0.05, 0.075, 0.09, 0.1, 0.2, 0.3, 0.5, 0.75, 0.9, 1.0%), and the like.

In some embodiments, a pharmaceutical composition is provided in a form that can be refrigerated and/or frozen. In some embodiments, a pharmaceutical composition is provided in a form that cannot be refrigerated and/or frozen. In some embodiments, reconstituted solutions and/or liquid dosage forms may be stored for a certain period of time after reconstitution (e.g., 2 hours, 12 hours, 24 hours, 2 days, 5 days, 7 days, 10 days, 2 weeks, a month, two months, or longer). In some embodiments, storage of compositions including an antibody agent for longer than the specified time results in degradation of the antibody agent.

Liquid dosage forms and/or reconstituted solutions may comprise particulate matter and/or discoloration prior to administration. In some embodiments, a solution should not be used if discolored or cloudy and/or if particulate matter remains after filtration.

General considerations in the formulation and/or manufacture of pharmaceutical agents may be found, for example, in Remington: The Science and Practice of Pharmacy 21st ed., Lippincott Williams & Wilkins, 2005.

### Kits

The present disclosure further provides a pharmaceutical pack or kit comprising one or more containers filled with at least one HLA-DR CAR and/or a nucleic acid encoding a HLA-DR CAR as described herein. Kits may be used in any applicable method, including, for example, therapeutic methods, diagnostic methods, cell proliferation and/or isolation methods, etc. Optionally associated with such container(s) can be a notice in the form prescribed by a governmental agency regulating the manufacture, use or sale of pharmaceuticals or biological products, which notice reflects (a) approval by the agency of manufacture, use or sale for human administration, (b) directions for use, or both.

In some embodiments, a kit may include one or more reagents for detection (e.g, detection of a HLA-DR CAR and/or a nucleic acid encoding a HLA-DR CAR. In some embodiments, a kit may include a HLA-DR CAR and/or a nucleic acid encoding a HLA-DR CAR in a detectable form (e.g., covalently associated with detectable moiety or entity).

In some embodiments, one or more HLA-DR CARs and/or a nucleic acids encoding a HLA-DR CAR as provided herein may be included in a kit used for treatment of subjects. In some embodiments, a HLA-DR CAR and/or a nucleic acid encoding a HLA-DR CAR as provided herein may be included in a kit used for preparing an autologous T cell expressing the HLA-DR CAR.

In some embodiments, a kit may provide one, two, three, four or more HLA-DR antibody agents, where each is suitable for cloning into a CAR construct. In some embodiments, a kit may provide other reagents for assaying binding affinity of a HLA-DR antibody agent (e.g., a MVR antibody agent) and/or HLA-DR CAR (e.g., MVR CAR) and/or a HLA-DR CAR T cell for a T cell or HLA-DR identified or isolated from a subject. In some embodiments, a kit may provide other reagents for assaying functional avidity of a HLA-DR antibody agent (e.g., a MVR antibody agent) and/or HLA-DR CAR (e.g., MVR CAR) and/or a HLA-DR CAR T cell for a T cell of a subject.

The contents of all cited references (including literature references, issued patents, published patent applications, and co-pending patent applications) cited throughout this application are hereby expressly incorporated by reference.

Other features of the invention will become apparent in the course of the following descriptions of exemplary embodiments. However, the following examples are merely provided to illustrate the present invention, but the scope of the present invention is not limited to the following examples.

### EXAMPLES

The present disclosure provides, at least in part, novel engineered T cells that express HL-DR CAR and methods related thereto. Generation and characterization of HLA-DR CAR-T compositions and methods of production and use are described in further detail in the following examples.

### Exemplary Methods

The following exemplary methods were used in the context of the Examples that follow, but methods that can be used in the context of the present invention are not limited thereto.

Plasmid Design

A DNA construct encoding a single-chain variable fragment (scFv) form of a MVR antibody agent (described in U.S. Patent Application Publication No. US 2016-0257762, which is herein incorporated by reference in its entirety) was generated by connecting the V_{L} and V_{H} regions with a GS linker using standard DNA cloning techniques provided in Table 1 below. A CD8α leader sequence was inserted at the 5′-terminal of the MVR-scFv sequence to allow the protein to be secreted (Table 1). For easier purification and detection, His-tag and FLAG-tag sequences were attached at the 5′- and 3′-terminals of the MVR-scFv sequence, respectively, using sequences as shown in Table 1 below:

TABLE 1
Sequences suitable for use in preparation of
exemplary constructs

| SEQ | | |
|---|---|---|
| ID | | |
| NO. | Name | Sequence |
| 10 | GS linker | GGGGSGGGGSGGGGS |
| 11 | CD8α leader | MALPVTALLLPLALLLHAARP |
| 12 | His-tag | HHHEIHH |
| 13 | FLAG-tag | DYKDDDDK |
| 14 | HLA-DRB1 exon3- | 5′-CAGGCAGCATTGAAGTCAGG-3′ |
| | targeting spacer | |
| 15 | CD8TM-BB_Fwd | 5′-GTTATCACCCTTTACTGCAAACG-3′ |
| 16 | BB-CD3z_Rev | 5′-CTCCTGCTGAACTTCACTCTCA-3′ |
| 17 | GAPDH_Fwd | 5′-TCGGAGTCAACGGATTTGGT-3′ |
| 18 | GAPDH_Rev | 5′-TTCCCGTTCTCAGCCTTGAC-3′ |

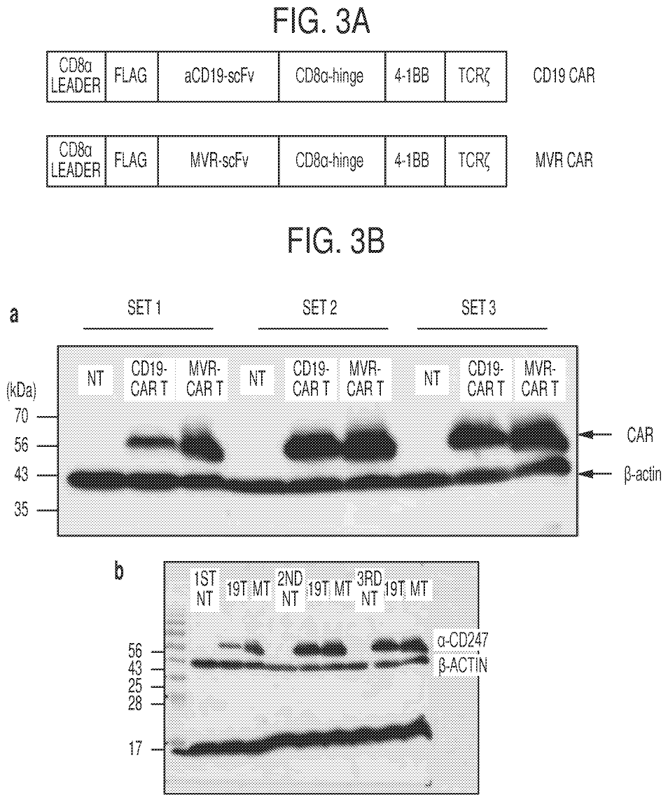

MVR-scFv was then cloned into a pcDNA3.1(+) expression vector (V790-20, Invitrogen, Carlsbad, Calif., USA) to generate pcDNA3.1-MVR-scFv. To create the MVR CAR construct, the MVR-scFv sequence was grafted into the previously described lentiviral vector pELPS-19BBz, which encodes a second-generation CD19 CAR construct (Milone, M. C. et al., (2009) Mol. Ther. 17: 1453-1464; June, C. et al., (2012) International Patent Publication No.: WO/2012/07900), using standard DNA cloning techniques. The FLAG-tag sequence was inserted between the CD8α leader and scFv sequences of CD19 CAR and MVR CAR to generate pELPS-FLAG19BBz and pELPS-FLAGMVRBBz, respectively (FIG. 3), so that expression of each construct could be detected in an unbiased manner with an anti-FLAG antibody. To generate pLCv2-DRB1, an HLA-DRB/-targeting sgRNA/Cas9 expression vector, the HLA-DRB1 exon3-targeting spacer sequence, was inserted into lentiCRISPRv2 (52961, Addgene, Cambridge, Mass., USA) using standard DNA cloning techniques (Table 1).

Cells and Media

PBMCs were obtained with informed consent from healthy volunteer donors at the National Cancer Center Research Institute using a National Cancer Center Institutional Review Board-approved protocol. PBMCs were isolated by density gradient centrifugation and either used immediately or stored in liquid nitrogen. EBV LCLs were generated from PBMCs by transformation with EBV. In detail, exponentially growing B95-8 cells were incubated for 3 days at 37° C. The supernatant was filtered through a 0.45-μm filter and used for transformation. For EBV-transformation, 10⁷ PBMCs in 2.5 mL media was mixed with 2.5 mL of EBV-containing supernatant and incubated for 2 h at 37° C. The mixed cells were transferred to a T75 flask, and 5 mL of media containing 1 μg/mL cyclosporine A was added. After 3 weeks of incubation, the outgrowing immortalized B cells were checked for CD19 and HLA-DR expression and used in the following Examples. The EBV LCL-lucH cell line was generated by single-cell cloning after electroporation of DR^{weak} EBV LCLs in the presence of the pGL4.51 vector (E132A, Promega, Madison, Wis., USA). ADR-EBV LCL, which has a defective HLA-DR molecule, was generated by introducing pLCv2-DRB1 into DR^{weak} EBV LCLs by electroporation. For electroporation, cells and plasmids were placed in 4-mm cuvettes and pulsed at 250 V, 975 μF with a Gene Pulser Xcell electroporation system (Bio-Rad Laboratories, Inc., Hercules, Calif., USA) using the exponential decay program. After electroporation, HLA-DR-negative DR^{weak} EBV LCLs were sorted with a FACSAria flow cytometer (BD Biosciences, Franklin Lakes, N.J., USA). 1A2 (CRL-8119, ATCC, Manassas, Va., USA), BC-1 (CRL-2230, ATCC), JVM-2 (CRL-3002, ATCC), Daudi (CCL-213, ATCC), Raji (CCL-86, ATCC), Ramos (CRL-1596, ATCC), NALM6 (CRL-3273, ATCC), B95-8 (CRL-1612, ATCC), EBV LCLs, EBV LCLs-lucH, and ADR-EBV LCLs were cultured in RPMI 1640 (LM011-01, Welgene, Inc., Daej eon, Korea) supplemented with 1% penicillin/streptomycin (15140-122, Gibco, Grand Island, N.Y., USA) and 10% heat-inactivated fetal bovine serum (FBS-BBT-5XM, Rocky Mountain Biologicals, Inc., Missoula, Mon., USA). Expanded T cells and PBMCs were cultured in RPMI 1640 (LM011-77, Welgene, Inc.) supplemented with 1% penicillin/streptomycin (15140-122, Gibco) and 10% heat-inactivated fetal bovine serum (FBS-BBT-5XM, Rocky Mountain Biologicals, Inc.). Lenti-X 293T (632180, Clontech Laboratories, Inc., Mountain View, Calif., USA) and 293T cell lines were cultured in DMEM (LM001-05, Welgene, Inc.) supplemented with 1% penicillin/streptomycin (15140-122, Gibco) and 10% heat-inactivated fetal bovine serum (FBS-BBT-5XM, Rocky Mountain Biologicals, Inc.). All cell lines used in the following examples were cultured in the presence of ZellShield (13-0050, Minerva Biolabs, Hackensack, N.J., USA) within the past year, and validated using an e-Myco VALiD Mycoplasma PCR Detection Kit (S25239, iNtRON Biotechnology, Inc., Seoul, Korea) to be free from mycoplasma. Cell line authentication was not conducted.

MVR-scFv Production

To produce purified MVR-scFv protein, pcDNA3.1-MVR-scFv was transfected into 293T cells. MVR-scFv protein secreted into the supernatant was collected at 48 h post-transfection and purified with a Ni-NTA Purification System (R901-10, Thermo Fisher Scientific, Inc., Waltham, Mass., USA) according to the manufacturer's protocol.

Flow Cytometry Methods and Antibodies

To analyze the expression of surface markers, 1×10⁶ cells were stained with specific antibodies for 30 min at 4° C. To assess the binding of MVR-scFv to surface receptors, 1×10⁶ cells were stained with 1 μg of purified MVR-scFv for 30 min at 4° C., washed once, and stained with PE- or APC-conjugated anti-FLAG antibody for 30 min at 4° C. The cells were washed twice and fixed with 1% paraformaldehyde before analysis. To analyze intracellular antigens, cells were stained with intracellular antigen-specific antibodies using a Cytofix/Cytoperm Fixation/Permeabilization Kit (554714, BD Biosciences). To evaluate proliferation after target antigen contact, T cells were labeled with a CellTrace violet cell proliferation kit (C34557, Thermo Fisher Scientific, Inc.) and EBV LCLs were y-irradiated at a dose of 30 Gy using a Gammacell 3000 ¹³⁷Cs irradiator (Best Theratronics, Ltd., Ontario, Canada). A total of 1.2×10⁶ cells were then mixed at a T cell:EBV LCL ratio of 3:1 and cultured for 5 days in the presence of 200 IU/mL of human recombinant IL-2. On day 5, the cultured cells were washed twice and fixed with 1% paraformaldehyde before analysis. Polyfunctionality was evaluated by measuring the levels of CD107a, IFN-γ, IL-2, MIP-1β, and TNF. EBV LCLs were labeled with a CellTrace carboxyfluorescein succinimidyl ester cell proliferation kit (C34554, Thermo Fisher Scientific, Inc.) and used to activate T cells. A total of 1.2×10⁶ cells were co-incubated at a T cell:EBV LCL ratio of 3:1 for 6 h in 48-well plates in the presence of a protein transport inhibitor cocktail (00-4980, Thermo Fisher Scientific, Inc.) and CD107a-specific antibody. The cells were stained with anti-CD4 antibody, washed twice, and stained intracellularly with IFN-γ-, IL-2-, MIP-1β-, and TNF-specific antibodies. All flow cytometric analysis was performed with FACSCalibur or FACSVerse flow cytometers (BD Biosciences). Further information regarding the antibodies used in the following examples is shown in Table 2 below.

TABLE 2
Exemplary antibodies suitable for use in the exemplary methods

| Specificity | Reactivity | Isotype | Conjugation | Clone | Manufacturer | Cat # | Dilution |
|---|---|---|---|---|---|---|---|
| CD3 | Human | Mouse | BV510 | HIT3a | BD Biosciences | 564713 | 1:20 |
| | | IgG2a, κ | | | | | |
| CD4 | Human | Mouse | PE | RPA-T4 | BD Biosciences | 555347 | 1:100 |
| | | IgG1, κ | | | | | |
| CD8 | Human | Mouse | FITC | RPA-T8 | BD Biosciences | 555366 | 1:100 |
| | | IgG1, κ | | | | | |
| CD14 | Human | Mouse | PE-Cy7 | MφP9 | BD Biosciences | 562698 | 1:50 |
| | | IgG2a, κ | | | | | |
| CD19 | Human | Mouse | PE | SJ25C1 | BD Biosciences | 340364 | 1:50 |
| | | IgG1, κ | | | | | |
| CD19 | Human | Mouse | APC | SJ25C1 | BD Biosciences | 340437 | 1:50 |
| | | IgG1, κ | | | | | |
| CD20 | Human | Mouse | APC-H7 | 2H7 | BD Biosciences | 560734 | 1:50 |
| | | IgG2b, κ | | | | | |
| CD20 | Human | Mouse | APC | 2H7 | BD Biosciences | 559776 | 1:50 |
| | | IgG2b, κ | | | | | |
| CD20 | Human | Mouse | FITC | 2H7 | BD Biosciences | 555622 | 1:50 |
| | | IgG2b, κ | | | | | |
| CD45 | Human | Mouse | PE-Cy5 | HI30 | BD Biosciences | 555484 | 1:50 |
| | | IgG1, κ | | | | | |
| CD223 | Human | Mouse | FITC | 3DS223H | Thermo Fisher | 11-2239-41 | 1:20 |
| (LAG-3) | | IgG1, κ | | | Scientific Inc. | | |
| CD366 | Human | Mouse | PE | F38-2E2 | Miltenyi Biotec | 130-098-960 | 1:20 |
| (TIM-3) | | IgG1 | | | Inc. | | |
| CD152 | Human | Mouse | PE-Cy5 | BNI3 | BD Biosciences | 555854 | 1:20 |
| (CTLA-4) | | IgG2a, κ | | | | | |
| CD279 | Human | Mouse | BV510 | EH12.1 | BD Biosciences | 563076 | 1:20 |
| (PD-1) | | IgG1, κ | | | | | |
| CD107a | Human | Mouse | BV510 | H4A3 | BD Biosciences | 563078 | 1:20 |
| | | IgG1, κ | | | | | |
| IFN-γ | Human | Mouse | PE-Cy7 | B27 | BD Biosciences | 557643 | 1:20 |
| | | IgG1, κ | | | | | |
| IL-2 | Human | Mouse | BV421 | 5344.111 | BD Biosciences | 562914 | 1:20 |
| | | IgG1, κ | | | | | |
| MIP-1β | Human | Mouse | APC-H7 | D21-1351 | BD Biosciences | 561280 | 1:20 |
| | | IgG1, κ | | | | | |
| TNF | Human | Mouse | PerCP-Cy5.5 | MAb11 | BD Biosciences | 560679 | 1:20 |
| | | IgG1, κ | | | | | |
| HLA-DR | Human | Mouse | PE | G46-6 | BD Biosciences | 555812 | 1:50 |
| | | IgG2a, κ | | | | | |
| HLA-DR | Human | Mouse | PE-Cy5 | G46-6 | BD Biosciences | 555813 | 1:50 |
| | | IgG2a, κ | | | | | |
| Granzyme A | Human | Mouse | PE | CB9 | BioLegend Inc. | 507206 | 1:20 |
| | | IgG1, κ | | | | | |
| Granzyme B | Human | Mouse | BV510 | GB11 | BD Biosciences | 563388 | 1:20 |
| | | IgG1, κ | | | | | |
| FLAG | — | Unknown | PE | Unknown | Miltenyi Biotec | 130-101-576 | 1:20 |
| (DYKDDDDK) | | | | | Inc. | | |
| FLAG | — | Unknown | APC | Unknown | Miltenyi Biotec | 130-101-564 | 1:20 |
| (DYKDDDDK) | | | | | Inc. | | |
| Isotype | — | Mouse | FITC | MOPC-31C | BD Biosciences | 550616 | 1:20 |
| control | | IgG1, κ | | | | | |
| Isotype | — | Mouse | PE | MOPC-31C | BD Biosciences | 550617 | 1:20 |
| control | | IgG1, κ | | | | | |
| Isotype | — | Mouse | PE-Cy5 | MOPC-31C | BD Biosciences | 550618 | 1:20 |
| control | | IgG1, κ | | | | | |
| Isotype | — | Mouse | PerCP-Cy5.5 | MOPC-21 | BD Biosciences | 550795 | 1:20 |
| control | | IgG1, κ | | | | | |
| Isotype | — | Mouse | PE-Cy7 | MOPC-21 | BD Biosciences | 557646 | 1:20 |
| control | | IgG1, κ | | | | | |
| Isotype | — | Mouse | APC | G155-178 | BD Biosciences | 550882 | 1:20 |
| control | | IgG2a, κ | | | | | |
| Isotype | — | Mouse | APC-H7 | MOPC-21 | BD Biosciences | 560167 | 1:20 |
| control | | IgG1, κ | | | | | |
| Isotype | — | Mouse | BV421 | X40 | BD Biosciences | 562438 | 1:20 |
| control | | IgG1, κ | | | | | |
| Isotype | — | Mouse | BV510 | X40 | BD Biosciences | 562946 | 1:20 |
| control | | IgG1, κ | | | | | |
| CD247 | Human | Mouse | unconjugated | 8D3 | BD Biosciences | 51-6527GR | 1:1000 |
| | | IgG1 | | | | | |
| IgG (H + L) | Mouse | Rabbit | HRP | polyclonal | Jackson | 315-035-045 | 1:10000 |
| | | IgG | | | ImmunoResearch | | |
| | | | | | Inc. | | |
| β-actin | Human | Rabbit | HRP | N-21 | Santa Cruz | sc-130656 | 1:1000 |
| | | IgG | | | Biotechnology | | |
| | | | | | Inc. | | |
| FLAG | — | Rabbit | AF488 | polyclonal | Cell Signaling | 5407 | 1:500 |
| (DYKDDDDK) | | Ig | | | Technology, | | |
| | | | | | Inc. | | |
| CD178 | Human | Mouse | unconjugated | NOK-1 | BD Biosciences | 556371 | 1:100 |
| | | IgG1 | | | | | |
| CD253 | Human | Mouse | unconjugated | RIK-2 | BD Biosciences | 550912 | 1:100 |
| | | IgG1 | | | | | |

Lentivirus Preparation

Lentivirus vectors were generated using Lenti-X 293 T packaging cell line and packaging plasmid vectors. On the day before transfection, Lenti-X 293T cells were seeded in a 150-mm culture dish at a density of 10⁵ cells/cm². The next day, on day 0, CAR-encoding lentivirus vector constructs (pELPS-FLAG19BBz and pELPS-FLAGMVRBBz) were transfected into Lenti-X 293T cells with packaging plasmid vectors, pMDLg/pRRE, pRSV-rev, and pMD.G, at a ratio of 16:7:7:1 using Lipofectamine 3000 (L3000075, Thermo Fisher Scientific, Inc.). Supernatants harvested 24 and 48 h post-transfection were concentrated by ultracentrifugation for 90 min at 16,500 xg at 4° C. in Thickwall Polyallomer tubes (355642, Beckman Coulter, Inc., Brea, Calif., USA). After ultracentrifugation, supernatants were discarded and 1 mL of fresh T cell media was added to each tube. Sealed tubes incubated overnight at 4° C. were filtered through a 0.45-μm filter and aliquoted and stocked at −70° C. until use. Lentivirus titers were determined by calculating transduction units. Human PBMCs were activated using a human T cell activation/expansion kit (130-091-441, Miltenyi Biotec, Inc., Bergisch Gladbach, Germany) on day 0. On day 2, T cells were seeded at a density of 10⁵ cells/well in 96-well flat-bottom plates in the presence of 50 μL T cell media. For transduction, 100 μL of a 3-fold serial-diluted lentivirus vector containing 10 μg/mL of polybrene was added to T cell-seeded wells and spinoculated for 2 h at 1,200 xg at 25° C. After spinoculation, the plate was incubated for 2 days at 37° C., and the transduced T cells were stained with anti-FLAG antibody and analyzed for CAR expression by FACSVerse flow cytometers (BD Biosciences). By determining the dilution rate, which resulted in a transduction rate between 0.05 and 0.1, transduction U/mL of lentivirus was calculated using the following equation: (transduction rate x 10⁵ x 10)/dilution rate.

CAR T Cell Production

CAR T cells were generated by spinoculation of activated T cells with CAR-encoding lentivirus. In detail, human PBMCs or T cells isolated using a pan T cell isolation kit (130-096-535, Miltenyi Biotec, Inc.) were activated using a human T cell activation/expansion kit (130-091-441, Miltenyi Biotec, Inc.) on day 0. On day 2, T cells were transduced with lentivirus at multiplicities of infection of 3-5 by 1,200 xg spinoculation for 2 h at 25° C. in media containing 10 μg/mL of polybrene. After spinoculation, the transduced T cells were washed and cultured in medium supplemented with 200 IU/mL of human recombinant IL-2 for 2 weeks. On day 14, CAR-expressing T cells were either used immediately or enriched using anti-FLAG-biotin (130-101-566, Miltenyi Biotec, Inc.) and anti-biotin microbeads (130-091-441, Miltenyi Biotec, Inc.) before use.

Quantitative PCR

CAR mRNA expression was determined by quantitative PCR. Total RNA from 1×10⁶ T cells was extracted using an RNeasy plus mini kit (74136, QIAGEN, Hilden, Germany) and reverse-transcribed using the SuperScript III first-strand synthesis system (18080-051, Thermo Fisher Scientific, Inc.). Reverse-transcribed single-stranded DNA was then subjected to quantitative PCR using a FastStart essential DNA green master kit and LightCycler 96 System (06924204001, Roche Molecular Systems, Inc., Basel, Switzerland). CD8TM-BB_Fwd (specific for the junction of the CD8α transmembrane with the 4-1BB signaling domain) and BB-CD3z_Rev (specific for the junction of 4-1BB with the CD3ζ signaling domain) were used to quantify CAR mRNA (Table 1). GAPDH_Fwd and GAPDH_Rev (specific for GAPDH mRNA) were used to detect reference gene expression (Table 1). CAR mRNA levels relative to GAPDH mRNA levels were calculated and used to compare CAR expression between CAR T cell samples.

Western Blot Analysis

To compare CAR protein levels, western blot analysis with a CD247-specific antibody (unconjugated; 51-6527GR, BD Biosciences; Table 2) was conducted. In detail, 1×10⁷ T cells were washed three times with ice-cold PBS and lysed with RIPA lysis buffer containing a protease inhibitor cocktail (P3100-001, GenDEPOT, Inc., Barker, Tex., USA). The lysates were centrifuged for 10 min at maximum speed at 4° C. and the supernatants were mixed with sample buffer (5×) and boiled for 5 min. Equal amounts of protein were separated on a 12% SDS-PAGE gel and transferred to a polyvinylidene fluoride membrane. The membrane was blocked for 1 h at 25° C. using 5% non-fat milk and incubated in the presence of anti-CD247 antibody overnight at 4° C. with gentle rocking. The membrane was then washed three times with TB S-T buffer and incubated with horseradish peroxidase-conjugated secondary anti-mouse IgG antibody (315-035-045, Jackson ImmunoResearch, Inc., West Grove, Pa., USA) and horseradish peroxidase-conjugated β-actin-specific antibody (sc-130656, Santa Cruz Biotechnology, Inc., Dallas, Tex., USA) for 1 h at 25° C. The membrane was washed three times with TBS-T buffer. For signal development, the membrane was developed with a chemiluminescent substrate (NCI4080KR, Thermo Fisher Scientific, Inc.) and exposed to X-ray film. The protein level of CAR relative to β-actin was quantified with ImageJ v1.50i software (NIH, Bethesda, Md., USA).

Immunofluorescence Imaging

CAR protein localization was assessed by immunofluorescence imaging. T cells were fixed in 4% (w/v) paraformaldehyde in PBS (pH 7.4) for 10 min at 25° C. Fixed cells were washed and permeabilized with perm-wash buffer (PBS, pH 7.4 containing 0.1% saponin and 1% bovine serum albumin) for 20 min at 25° C. and blocked with human Fc Block (564219, BD Biosciences) for 20 min at 25° C. After washing with perm-wash buffer, the cells were stained with Alexa488-conjugated anti-FLAG-tag antibody (5407, Cell Signaling Technology, Inc., Danvers, Mass., USA; Table 2) in perm-wash buffer for 30 min at 25° C. The cells were washed in perm-wash buffer and mounted on glass slides using Vectashield mounting medium containing DAPI (H-1200, Vector Laboratories, Inc., Burlingame, Calif., USA) and images were acquired using a Zeiss LSM 780 laser scanning confocal microscope (Carl Zeiss SAS, Oberkochen, Germany).

Assessment of Cytotoxicity

Cytotoxic killing of EBV LCLs by T cells was quantified using the CytoTox-Glo cytotoxicity assay kit (G9291, Promega, Madison, Wis., USA). In detail, 5×10⁴ EBV LCLs were seeded in 96-well black plates with transparent flat bottoms (3904, Corning, Inc., Corning, N.Y., USA). T cells were then added to the wells at T cell:EBV LCL ratios of 1:27, 1:9, 1:3, 1:1, or 3:1 and incubated for 4 h at 37° C. Control wells containing EBV LCLs alone were incubated under the same conditions. After incubation, luminogenic AAF-Glo Substrate was added to each well, and luminescence was measured with a TECAN infinite PRO 200 (Tecan Group, Ltd., Mannedorf, Switzerland). Wells containing either EBV LCLs alone or digitonin-treated EBV LCLs were used as controls to detect background and maximum cytotoxicity signals, respectively. Cytotoxicity-induced killing efficacy was determined using the following equation: (cytotoxicity signal in sample well—background cytotoxicity signal)/maximum cytotoxicity signal.

Assessment of In Vitro On-Target Killing

To evaluate the target-specific killing efficacy of CAR T cells, a flow cytometry-based killing assay was designed. In detail, PBMCs and EBV LCLs were labeled with a CellTrace violet cell proliferation kit (C34557, Thermo Fisher Scientific, Inc.) and CellTrace carboxyfluorescein succinimidyl ester cell proliferation kit (C34554, Thermo Fisher Scientific, Inc.), respectively. Labeled PBMCs and EBV LCLs were co-cultured with T cells at a T cell:EBV LCL:PBMC ratio of 6:1:1 for 4 h. For co-culture, 1.2×10⁶ cells were incubated in the wells of 48-well plates in 1 mL of medium. Control wells contained labeled EBV LCLs and PBMCs only to measure the decrease in target cells in the absence of T cells. After incubation, 20 μL of Flow-Count fluorospheres (7547053, Beckman Coulter, Inc.) were added to each well for quantitative flow cytometric analysis. The cell-bead mixtures were then transferred into 12 x 75-mm polystyrene tubes and stained with the fixable viability dye eFluor 780 (65-0865, Thermo Fisher Scientific, Inc.), and with antibodies specific for HLA-DR, CD14, and CD20. The samples were then fixed with 1% paraformaldehyde and analyzed with a FACSVerse flow cytometer (BD Biosciences). For quantitative population analysis, a fixed number of quantitative beads was acquired from all samples. The killing efficacies of T cells against carboxyfluorescein succinimidyl ester-labeled EBV LCLs and violet-labeled CD20-positive B cells were calculated using the following equation: EBV LCL-killing efficacy=(live EBV LCLs in control well—live EBV LCLs in sample well)/live EBV LCLs in control wells; B cell killing efficacy=(live B cells in control well—live B cells in sample well)/live B cells in control wells.

Assessment of Cytotoxicity Inhibition

The cytotoxicity inhibition assay was performed as in the in vitro on-target killing assay with some modifications. Briefly, EBV LCLs were labeled using a CellTrace violet cell proliferation kit (C34557, Thermo Fisher Scientific, Inc.) and co-cultured with each type of T cell at a T cell:EBV LCL ratio of 5:1 for 4 h in the presence of anti-CD178 (FasL) antibody (FasL blocker; unconjugated; 10 μg/mL; 556371, BD Biosciences; Table 2), anti-CD253 (TRAIL) antibody (TRAIL blocker; unconjugated; 10 μg/mL; 550912, BD Biosciences; Table 2), concanamycin A (CMA; perforin-1 blocker; 1 μg/mL; C9705-25UG, Sigma-Aldrich, St. Louis, Mo., USA), or recombinant human Bcl-2 Protein (granzyme B blocker; 1 μg/mL; 827-BC, R&D Systems, Minneapolis, Minn., USA). Samples of 1.2×10⁶ cells were co-cultured in 48-well plates with 0.5 mL media. A T cell-EBV LCL mixture, containing 10 μg/mL of isotype mouse IgGs and 1 μg/mL of dimethyl sulfoxide, was used as a non-inhibited control. Labeled EBV LCLs alone were used as background controls. After incubation, 20 μL of Flow-Count fluorospheres (7547053, Beckman Coulter, Inc.) were added directly to each well for quantitative flow cytometric analysis. The cell-bead mixtures were then transferred to 12×75-mm polystyrene tubes and stained with fixable viability dye eFluor780 (65-0865, Thermo Fisher Scientific, Inc.), and then fixed with 1% paraformaldehyde and analyzed using a FACSVerse flow cytometer (BD Biosciences). For quantitative analysis, a fixed number of quantitative beads were acquired from all samples. The efficiency of inhibited EBV LCL killing was determined using the following equation: (EBV LCLs in reagent-containing sample—EBV LCLs in non-inhibited controls)/(EBV LCLs in background control—EBV LCLs in non-inhibited controls).

Quantification of Surface Molecules

Surface molecules were quantified using a Quantum Simply Cellular anti-Mouse IgG kit (814, Bangs Laboratories, Inc., Fishers, Ind., USA). APC-conjugated FLAG-specific antibodies, PE-conjugated CD19-specific antibodies, and PE-Cy5-conjugated HLA-DR-specific antibodies were used to quantify CAR, CD19, and HLA-DR, respectively. Flow cytometric analysis was performed using a FACSVerse flow cytometer (BD Biosciences).

Measurement of Granule Transfer Rates

Granule transfer rates following contact between T cells and B cells (or EBV LCLs) were measured by flow cytometry. First, T cells were labeled with a CellTrace violet cell proliferation kit (C34557, Thermo Fisher Scientific, Inc.). EBV LCLs or B cells from the PBMCs of healthy donors isolated using a B cell isolation kit II (130-091-151, Miltenyi Biotec, Inc.) were used as target cells. Samples of 4.5×10⁵ T cells and target cells in a T cell:target cell ratio of 2:1 were incubated for 10, 30, or 90 min in 96-well flat bottom plates. After incubation, the cells were fixed and permeabilized with a Cytofix/Cytoperm Fixation/Permeabilization kit (554714, BD Biosciences) and transferred granules were stained with anti-granzyme A and anti-granzyme B antibodies and analyzed by FACSVerse flow cytometer (BD Biosciences). The target cells were identified by gating on violet-negative cells. The granule-transfer rate was calculated from the percentage of granzyme A and/or granzyme B-positive cells among the total target cells.

Live Imaging of Apoptotic Cells

The kinetics of EBV LCL apoptosis were measured with a JuLI Stage real-time cell history recorder (NanoEnTek, Inc., Gyeonggi-do, Korea). Target EBV LCLs were labeled with a CellTrace violet cell proliferation kit (C34557, Thermo Fisher Scientific, Inc.). Samples of 1×10⁵ T cell and EBV LCL at a T cell:EBV LCL ratio of 1:1 were incubated in 96-well flat-bottom plates in the presence of IncuCyte caspase-3/7 reagent to induce apoptosis (4440, Essen BioScience, Ann Arbor, Mich., USA). DAPI- and RFP-filtered images were taken every 5 min for 90 min. Three areas of each well were analyzed. Because of the blue fluorescence of violet-labeled EBV LCLs, apoptotic EBV LCLs can be identified by observing magenta-colored cells in merged images (blue fluorescence of violet label combined with red fluorescence of apoptotic cells). The percentage of apoptotic EBV LCLs was determined and converted into a numerical value with ImageJ v1.50i software and JuLI STAT (NanoEnTek, Inc.). The proportion of apoptotic EBV LCLs was calculated from the equation: % apoptotic EBV LCLs=apoptotic EBV LCLs (magenta colored)/total EBV LCLs (blue or magenta colored).

Animal Models

For animal experiments described in the following examples, immune-deficient 7-10-week-old C; 129S4-Rag2^{tm1.1Flv}Il2rg^{tm1.1Flv}/J female mice kept under specific-pathogen-free conditions were used. Mice were sacrificed by carbon dioxide exposure when tumor volume exceeded 2,000 mm³ or the total luminescence of luciferin-treated subject exceeded 1×10¹¹ photons/s.

Assessment of In Vivo Efficacy

In vivo CAR T cell efficacy was evaluated using a xenograft model. Five days before T cell infusion, mice were intraperitoneally xenografted with 3×10⁶ (100 μL) luciferase-expressing EBV LCL-lucH cells. After 5 days (on day 0), 5×10⁶ T cells (300 μL) were injected intravenously per mouse. Four mice were injected with NT T cells, and five mice were injected with CD19 CAR T and MVR CAR T cells, respectively. The tumor burdens of the xenografted mice were determined on days 0, 7, 14, 21, and 28 by measuring luciferase activity with an IVIS Lumina in vivo imaging system (PerkinElmer, Inc., Waltham, Mass., USA).

Assessment of In Vivo On-Target Killing

A transient xenograft model was used for assaying in vivo on-target killing. In detail, 1 mg of clodronate liposomes (ClodLip BV, Amsterdam, Netherlands) was injected intravenously into mice 5 days before infusion with T cells. The next day, the mice were X-ray irradiated with a dose of 2 Gy using X-RAD 320 (Precision X-Ray, Inc., North Branford, Conn., USA), and intravenously grafted with 3×10⁵ (300 μL) DR^{weak} B cells from DR^{weak} PBMCs obtained with a B cell isolation kit II (130-091-151, Miltenyi Biotec, Inc.). Three days before T cell infusion, 6.5×10⁵ (200 μL) of luciferase-expressing EBV LCL-lucH cells were injected intraperitoneally into the mice. After 3 days (on day 0) 1×10⁷ T cells (500 μL) were injected intravenously per mouse. Four mice were injected with NT T and MVR CAR T cells, respectively, and five mice were injected with CD19 CAR T cells. All xenografted mice were analyzed for tumor burden on days −1, 7, and 14 by measuring luciferase activity with the IVIS Lumina in vivo imaging system. The persistence of B cells and blood IFN-γ levels were measured in blood samples collected by retro-orbital bleeding on day −1, 2, and 7. To quantify the remaining B cells in blood samples, CD3-, CD20-, CD45-, and HLA-DR-specific antibodies were added directly to 75 μL of EDTA-treated peripheral blood. After staining, red blood cell lysis buffer was added and the samples were transferred into 12×75-mm polystyrene tubes. Flow-Count fluorospheres (7547053, Beckman Coulter, Inc.) were added to each well for quantitative flow cytometric analysis. The cell-bead mixtures were then washed twice and fixed with 1% paraformaldehyde, and analyzed by FACSVerse flow cytometry. For quantitative population analysis, a fixed number of quantitative beads were acquired from all samples. IFN-γ levels in plasma collected from centrifuged blood samples were quantified with a BD Cytometric Bead Array human Th1/Th2/Th17 cytokine kit (560484, BD Biosciences).

Statistical Analyses

Statistical tests appropriate for the data based on similar studies in the field were used. Unpaired two-tailed t-tests were used to evaluate differences unless otherwise specified. p<0.05 was considered statistically significant and significance is designated with asterisks (ns, not significant; *,p<0.05; **,p<0.01; ***,p<0.001). Prism v5.01 (GraphPad Software, Inc., La Jolla, Calif., USA) was used to generate all graphs and for all statistical analyses.

### Example 1—Low CAR Affinity Reduces Fratricide of Exemplary HLA-DR CAR T Cells

This example describes HLA-DR CAR T cells with varying affinity to HLA-DR antigens from different subjects. Moreover, this example demonstrates that HLA-DR CAR T cells that were engineered with an HLA-DR CAR that has low affinity to a T cell from a subject has certain beneficial properties.

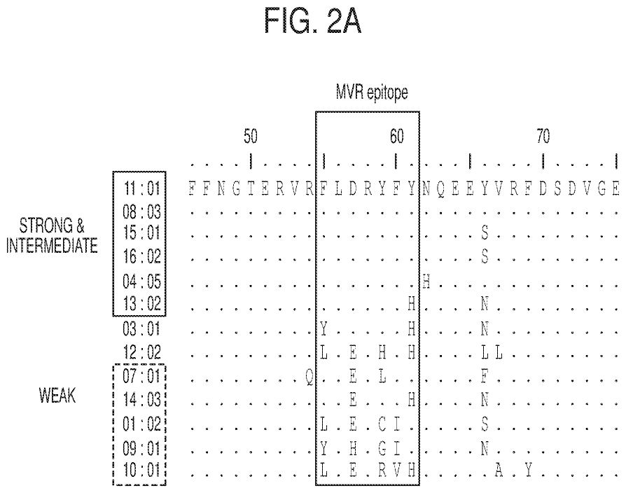

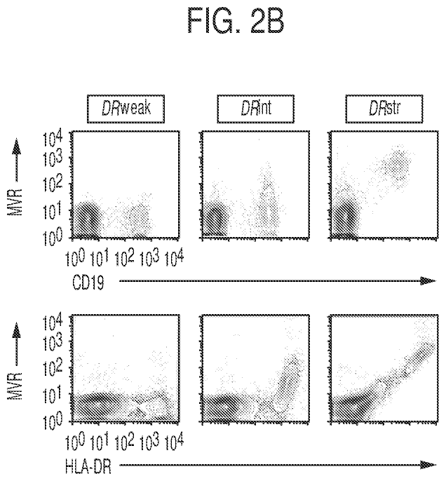

Recently, our group developed a HLA-DR-specific antibody agent, MVR, by immunizing mice with B cell lymphoma cell line, L3055. This exemplary HLA-DR antibody agent recognizes a polymorphic region of HLA-DR (described in U.S. Patent Application Publication No. US 2016-0257762, which is herein incorporated by reference in its entirety). Interestingly, because an MVR antibody agent recognizes a polymorphic region of HLA-DR, PBMCs from individuals of different HLA-DRB1 backgrounds can exhibit a broad spectrum of MVR-scFv binding affinities (not published). Provided in FIG. 2A is a sequence alignment of a polymorphic region of HLA-DR, with the MVR epitope region indicated. Exemplary CD19⁺ B cells from three donors were found to bind to an exemplary HLA-DR-scFv, MVR-scFv, with high (strong), middle (intermediate), or low (weak) affinity (respectively named as DR′, DR′, or DR^{weak}), and cells from these donors were used for further experiments (FIG. 2B). Exemplary sequence variation of a HLA-DR polymorphic region for strong/intermediate and weak binders is also depicted in the sequence alignment in FIG. 2A.

Recently, one group reported fratricide of CAR T cells redirected against CD5 which is expressed on T cells (Mamonkin, M., et al., (2015) Blood 126: 983-992). In the study, fratricide resulted in 2 to 3 days delayed expansion than CD19-CAR T cells at initial stage of in vitro culture (˜2 weeks post transduction), and recovery was observed at further stage of culture (2˜4 weeks post transduction) that was associated with an increased number of CD5^{low} T cells.

HLA-DR, the target antigen of MVR-scFv, is mainly expressed in antigen presenting cells (APCs). However, T cell activation induces an up-regulation HLA-DR in these cells. Because activated T cells express HLA-DR on their surface, T cells transduced with an HLA-DR CAR, such as an MVR CAR, were hypothesized to continuously recognize HLA-DR and induce fratricide and CAR downregulation.

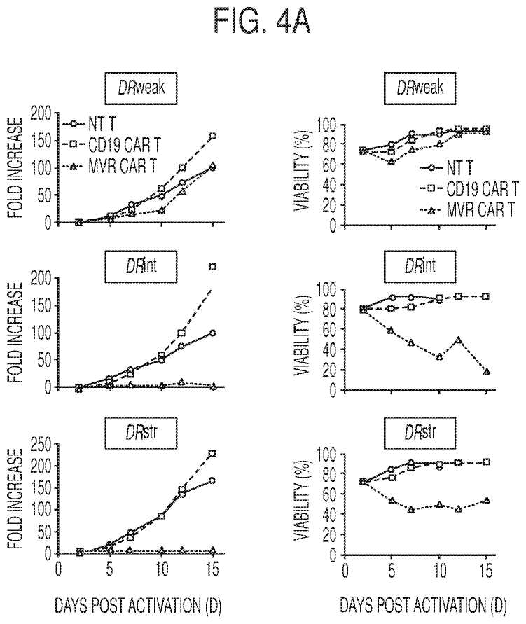

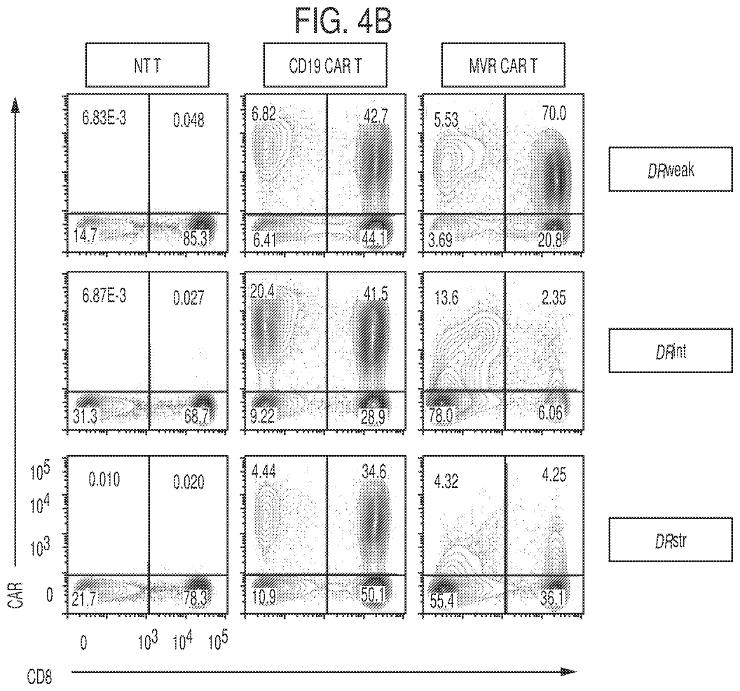

HLA-DR-targeted CAR T cells were engineered from T cells with different HLA-DRB1 variants (e.g., T cells from subjects that are characterized as having strong, intermediate, and/or weak binding to a HLA-DR antibody agent or HLA-DR CAR). DR′, DR′, and DR^{weak} T cells were transduced with a second-generation MVR CAR construct (FIG. 3A). Fratricidal degrees of second-generation MVR-CAR-transduced T cells with HLA-DRB1 variants characterized as DR^{str}, DR^{int}, and DR^{weak} PBMCs were evaluated for the extent of fratricide and CAR downregulation as a function of CAR-antigen affinity. CD19-targeted CAR T (CD19 CAR T) cells and non-transduced T (NT T) cells were generated as controls. Growth rates and viability of DR^{str} and DR^{int} MVR CAR T cells were assessed. Both DR^{str}- and DR^{int}-CAR T cell growth rates and viabilities decreased from the day after transduction (FIG. 4A). In contrast, DR^{weak} MVR CAR T cells continued to grow in a similar manner to parental NT T cells (FIG. 4A). Moreover, the frequency of MVR CAR-positive cells was profoundly decreased in DR^{str} and DR^{int} MVR CAR T cells, implying the interaction between MVR-CAR and HLA-DR is involved in the fratricidal cell death (FIG. 4B).

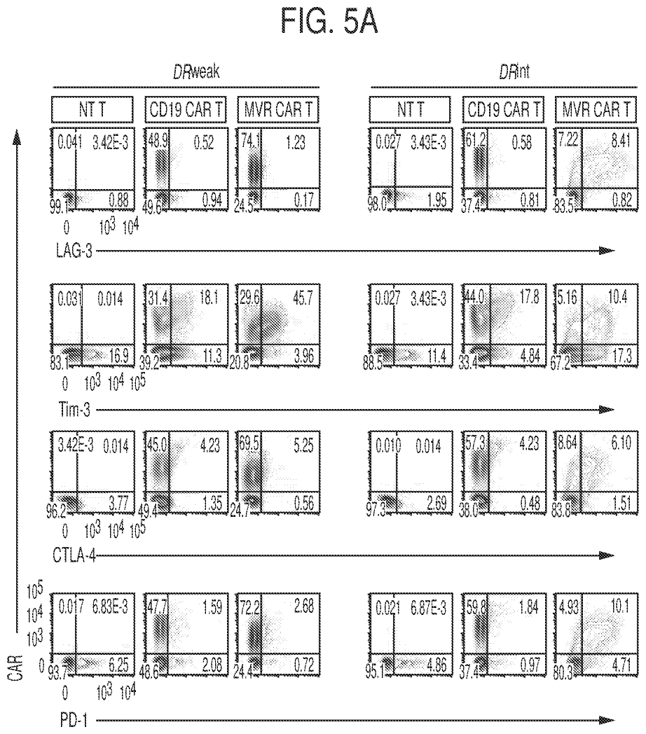

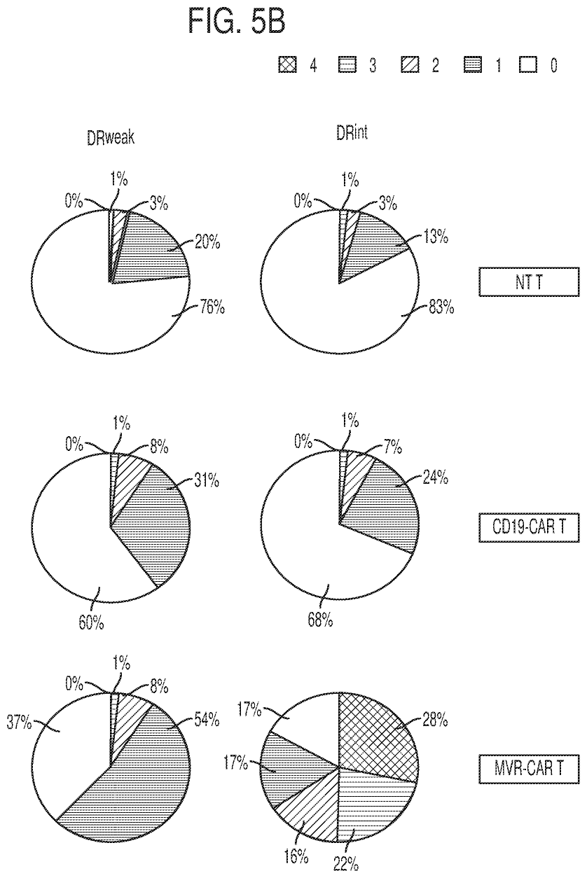

Similarly to TCR-mediated exhaustion, continuous CAR signaling gives rise to T cell exhaustion and related T cell dysfunction. (Long, A. H. et al. (2015) Nat. Med. 21: 581-590; Frigault, M. J. et al. (2015) Cancer Immunol. Res. 3: 356-367). Even though DR^{weak}-CAR T cells exhibited minimal fratricide, it was unclear whether the interaction between MVR-CAR and DR^{weak}-HLA-DR during the in vitro expansion would still give rise to T cell exhaustion and/or other related T cell dysfunctions. To assess the extent of exhaustion in these cells, expression of representative exhaustion markers, LAG-3, TIM-3, CTLA-4, and PD-1(Wherry, E. J. & Kurachi, M. (2015) Nat. Rev. Immunol. 15: 486-499; Blackburn, S. D. et al., (2009) Nat. Immunol. 10: 29-37; Speiser, D. E., et al., (2016) Nat. Rev. Immunol. 16: 599-611), was examined in DR^{int} and DR^{weak} MVR CAR T cells. DR^{weak} MVR CAR T cells did not display strong exhaustion and rarely expressed multiple exhaustion markers simultaneously (FIG. 5A and FIG. 5B). In contrast, most DR^{int} MVR CAR T cells (e.g., more than half), expressed two or more representative exhaustion markers (FIG. 5A and FIG. 5B).

A high proportion of DR^{int}-CAR T cells expressed two or more exhaustion markers, while CD19-CAR T cells from DR^{int}-PBMCs did not (MVR-CAR=65.8%, CD19-CAR=7.7%; FIG. 5B). Interestingly, DR^{weak}-CAR T cells exhibited only slight increase of Tim-3 than CD19-CAR T cells from DR^{weak}-PBMCs (MVR-CAR=60.7%, CD19-CAR=36.6%) while the frequency of CAR T cells with two or more exhaustion markers was similar (MVR-CAR=9.2%, CD19-CAR=9.4%). These data show that fratricide and exhaustion caused by the interaction of MVR-CAR and HLA-DR are minimal and tolerable in D^{weak}-CAR T cells, whereas fratricide and exhaustion severe and essentially unrecoverable in DR^{str}- and DR^{int}-CAR T cells. These data indicate that fratricide and exhaustion caused by the interaction of MVR CAR and HLA-DR are minimal in DR^{weak} MVR CAR T cells, whereas they are severe in DRS and DR^{int} MVR CAR T cells.

This example demonstrates that sensitivity selection of T cells was mimicked by fratricide. Moreover, these results demonstrate that in contrast to DR^{str} MVR CAR T cells, DR^{weak} MVR CAR T cells exhibited mild fratricide and exhaustion, indicating that a low affinity interaction between MVR CAR and DR^{weak} HLA-DR can induce a limited immune response. Indeed, DR^{weak} MVR CAR T cells were not cytotoxic to DR^{weak} B cells, while they killed DR^{str} B cells. These results suggest that fratricidal selection can be a useful strategy for CAR T cell development in which potentially harmful CAR T cells are detected and removed.

MVR CAR T cells used in the following example sections are DR^{weak} MVR CAR T cells, unless otherwise specified.

### Example 2—CAR-HLA-DR Interaction Downregulates Surface MVR CAR

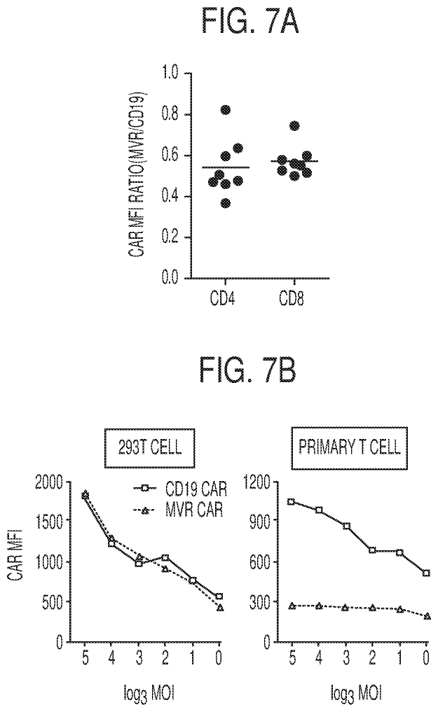

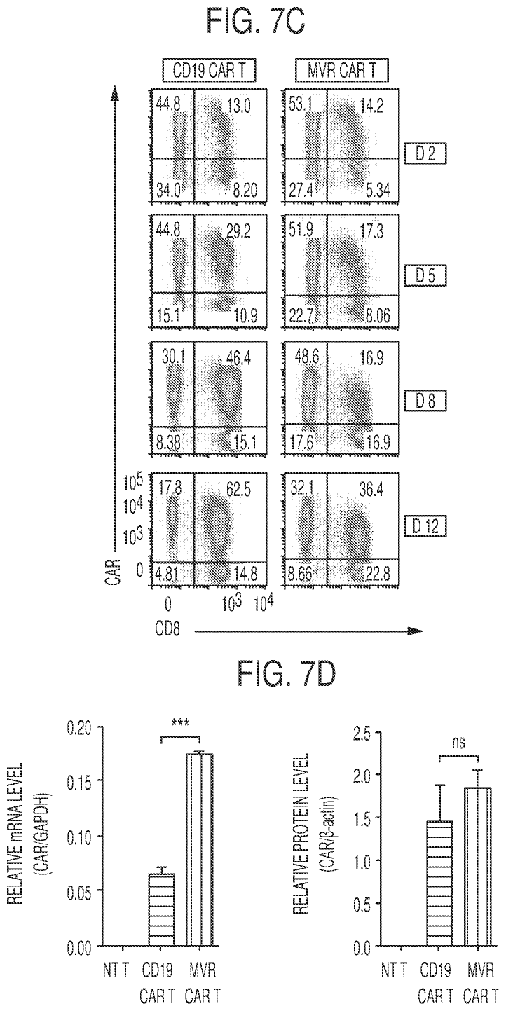

This example describes surface expression of HLA-DR CAR in T cells. While DR^{str} and DR^{int} MVR CAR T cells exhibited heavy downregulation of CAR (FIG. 4B), DR^{weak} MVR CAR T cells exhibited approximately 2-fold lower surface CAR expression than CD19 CAR T cells (FIG. 4B, FIG. 7A). This difference was confirmed in 293T cell lines and primary DR^{weak} T cells transduced with various multiplicities of infection of a MVR CAR or a CD19 CAR lentiviral vectors (FIG. 7B). Although surface expression of a MVR CAR increased with the multiplicity of infection in a 293T cell line (left panel), expression in primary DR^{weak} T cells remained essentially constant (right panel) (FIG. 7B).

Longitudinal analysis of CAR expression revealed that DR^{weak} T cells expressing the highest levels of surface MVR CAR were present 2 days post-transduction (4 days post-activation), and MVR CAR was gradually downregulated over the 14 days of the T cell activation cycle (FIG. 7C). CAR mRNA and protein levels in DR^{weak} MVR CAR T cells were similar to or higher than in CD19 CAR T cells, indicating that surface CAR is downregulated post-translationally (FIG. 7D, FIG. 3B).

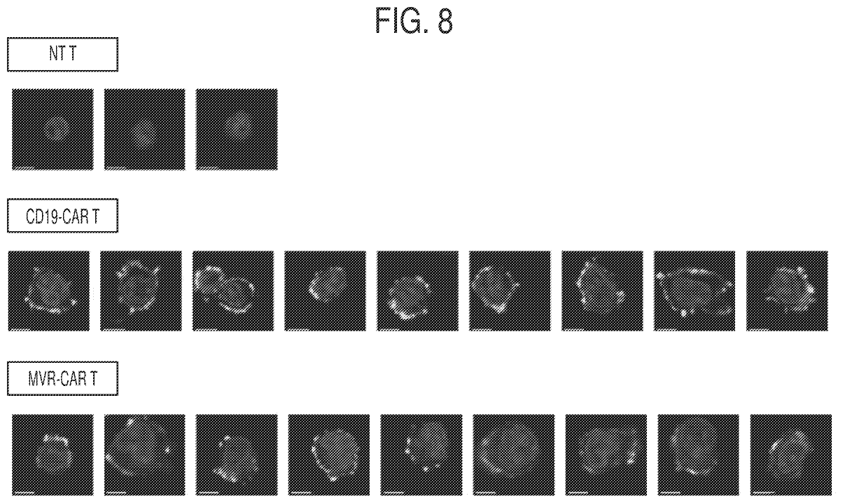

To determine if downregulation of MVR CAR was induced by the interaction of MVR CAR with HLA-DR, repeated attempts were made to generate a HLA-DR-deficient MVR CAR T cells using the CRISPR-Cas9 system. However, these attempts repeatedly failed, possibly because of an unknown survival advantage of HLA-DR in T cells. Due to the higher HLA-DR expression profile on malignant B cells, we presumed that despite the tolerable immune activation by DR^{weak}-CAR T cells themselves, model malignant cells, EBV-LCLs, may induce proper immune activation. We therefore generated Epstein-Barr virus-induced lymphoblastoid cell lines defective in HLA-DR (ΔDR-EBV LCLs) and transduced these cells with MVR CAR lentivirus. ADR-EBV LCLs expressed higher levels of MVR CAR than DR^{weak} EBV LCLs, and expression decreased after contact with DR^{weak} EBV LCLs, suggesting that the MVR CAR-HLA-DR interaction is responsible for MVR CAR downregulation (not shown). Further immunofluorescence experiments indicated that CAR was localized on the membrane in DR^{weak} MVR CAR T cells and CD19 CAR T cells (FIG. 8). These data suggest that sustained downregulation of surface MVR CAR occurs during in vitro expansion of DR^{weak} MVR CAR T cells because of the interaction with HLA-DR.

Thus, this example demonstrated that sensitivity selection analogous to that which is observed with TCR, can be mimicked in CAR T cells by fratricide. DR^{str} and DR^{int} MVR CAR T cells were involved in substantial fratricide, as the affinity between the MVR CAR and the HLA-DRs was sufficiently high to induce strong immune activation. Intense immune activation was inferred from the elevated exhaustion level of DR^{str} MVR CAR T cells (FIG. 5A and FIG. 5B). In contrast, DR^{weak} MVR CAR T cells exhibited mild fratricide and exhaustion, indicating that the affinity between MVR CAR and DR^{weak} HLA-DR was sufficiently low to limit the immune response. Indeed, DR^{weak} MVR CAR T cells were not cytotoxic to DR^{weak} B cells, while they killed DR^{str} B cells. Thus, DR^{weak} MVR CAR T cells can survive fratricidal selection and downregulate CAR on their surface. The present disclosure encompasses a recognition that fratricidal selection may be a useful strategy for CAR T cell development in which potentially harmful CAR T cells are detected and removed.

### Example 3—Hla-Dr Car T Cells Kill Malignant Cells while Sparing Normal B Cells

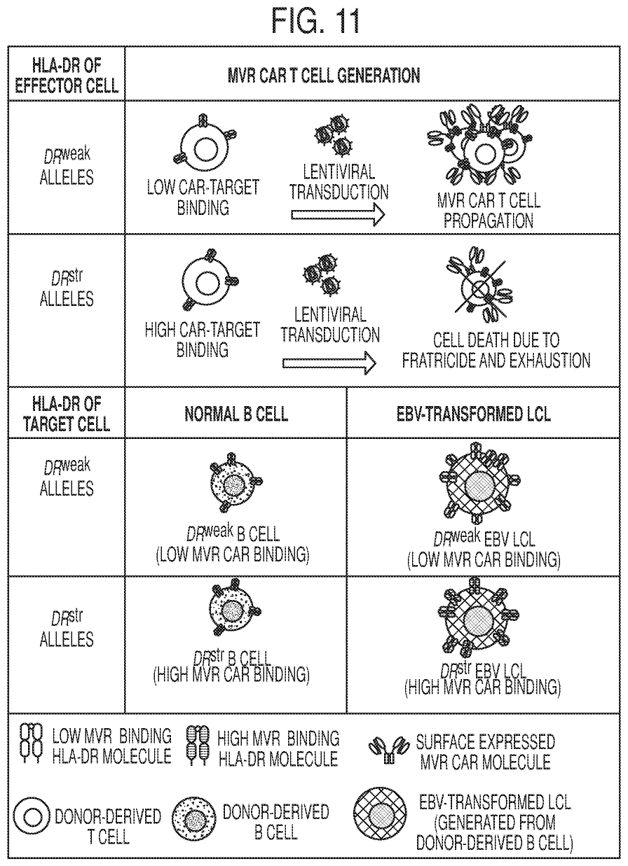

This example describes analysis of the functional consequences of fratricidal selection and CAR downregulation by comparing the immune activation capacity of CD19 CAR T and DR^{weak} MVR CAR T cells. EBV LCLs continuously expressing CD19 and HLA-DR were used for activation. To match the HLA-DRB1 alleles of DR^{weak} MVR CAR T cells and target cells, EBV LCLs were generated by EBV transformation of DR^{weak} B cells. Accordingly, the functional activities of CD19 CAR T and DR^{weak} MVR CAR T cells were compared against DR^{weak} EBV LCLs (FIG. 11). DR^{str} EBV LCLs, whose HLA-DRs bind strongly to MVR CAR and hence induce strong immune activation, served as positive controls.

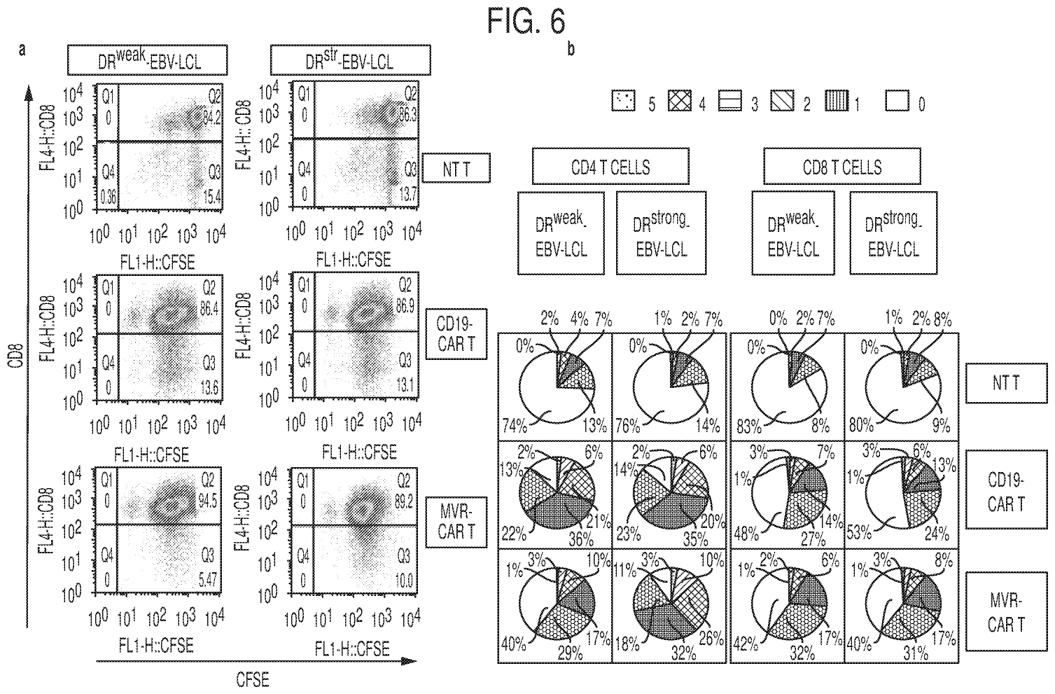

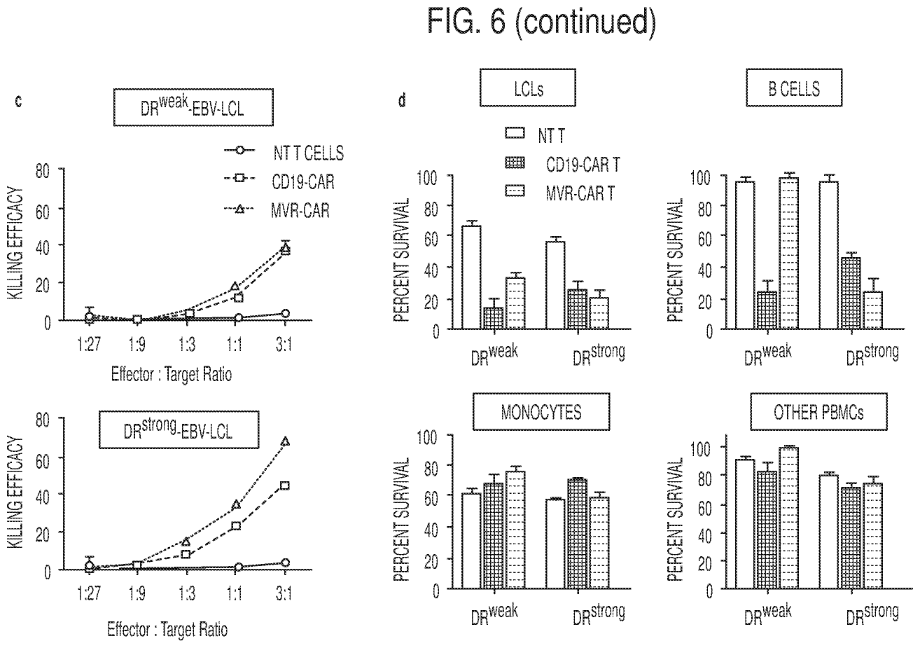

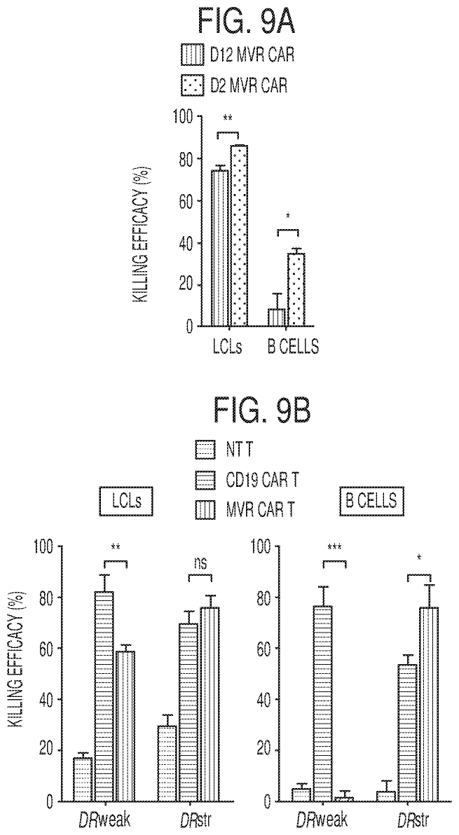

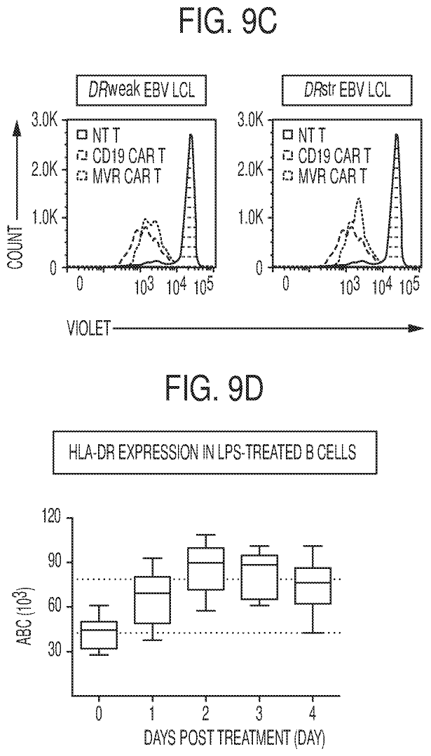

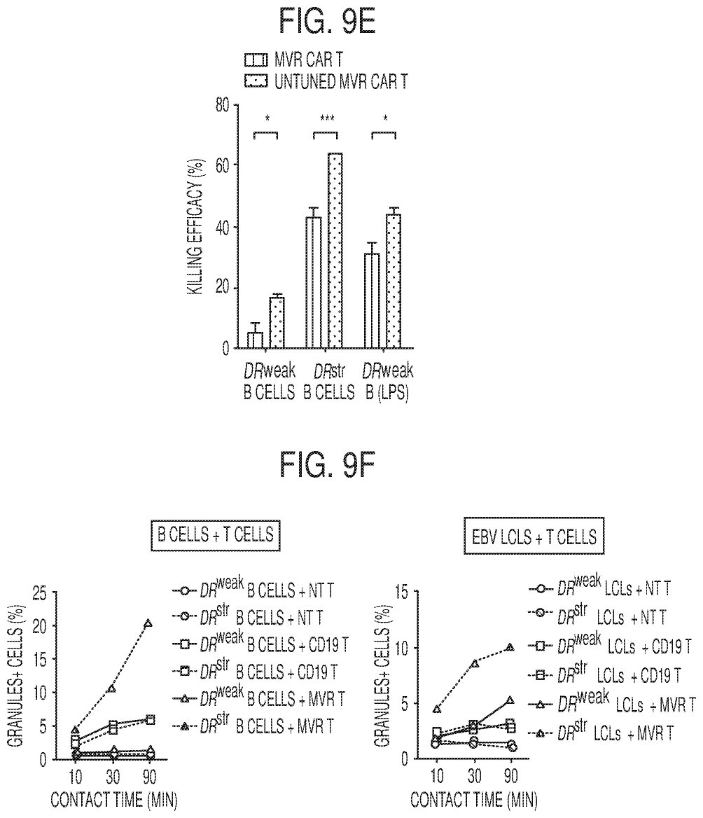

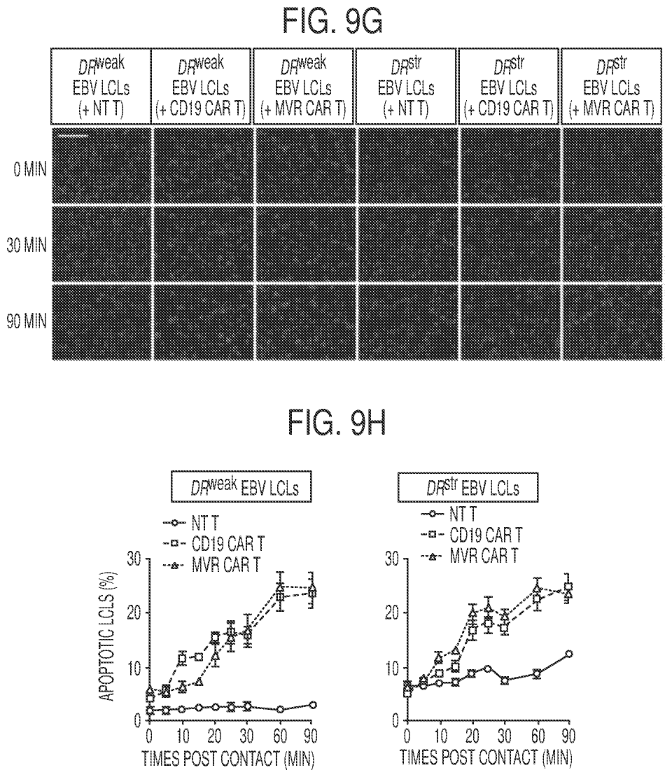

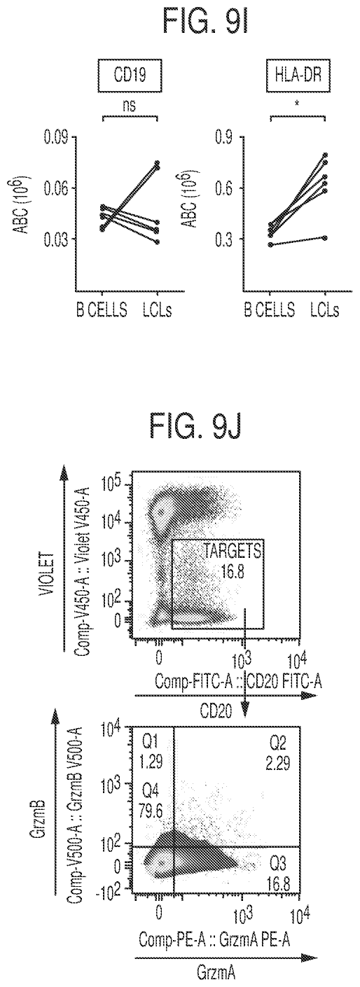

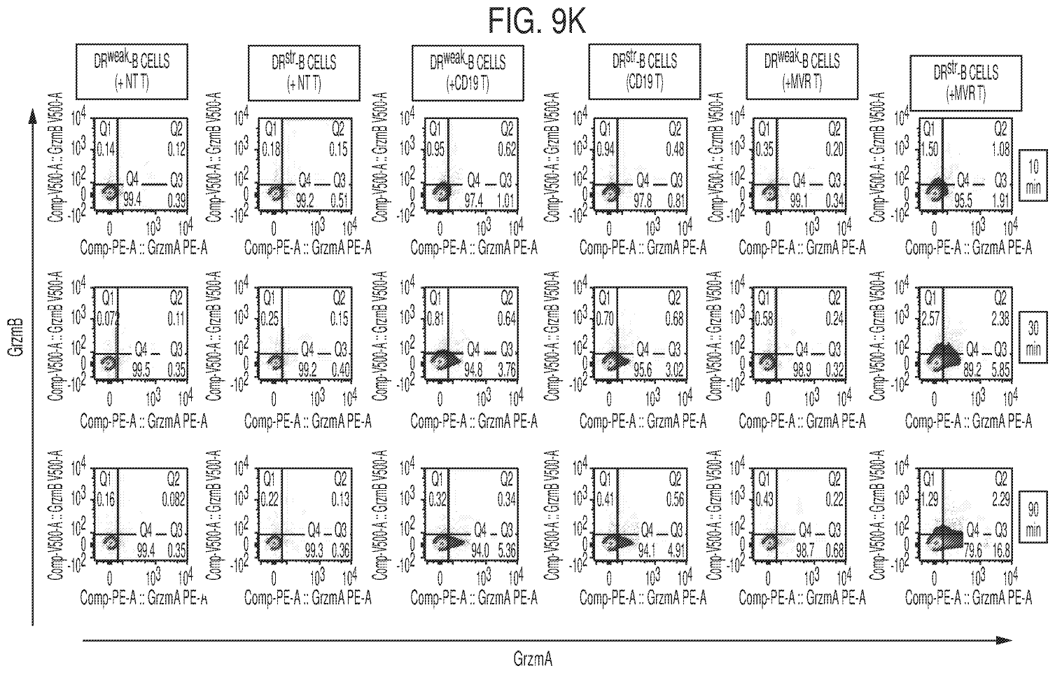

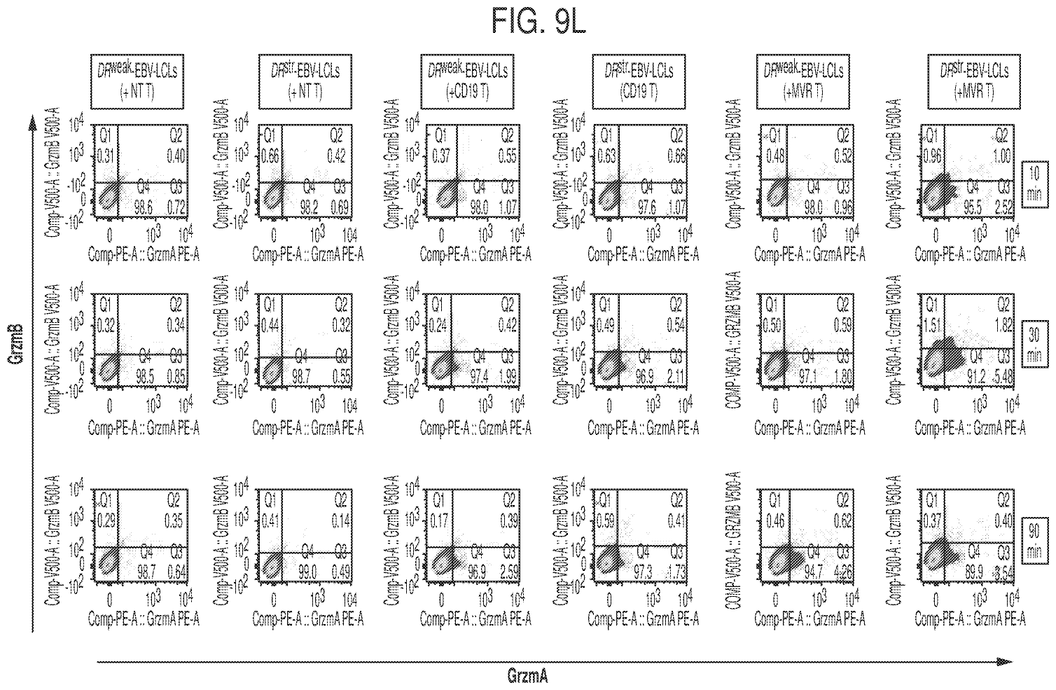

Proliferation is one of the representative features of T cell activation. To assess proliferative potential of MVR-CAR T cells following contact upon activation, HLA-DR CAR T cells were co-cultured with an exemplary malignant cell line. Specifically, MVR-CAR T cells were co-cultured with with Epstein-Barr virus-induced lymphoblastoid cell line (EBV-LCL) cells with HLA-DR variants of different binding affinities, EBV-LCLs DR^{weak}- or DR^{str}-EBV-LCLs. Interestingly, MVR-CAR T cells exhibited similar proliferation asCD19-CAR T cells following DR^{weak}-EBV-LCLs contact (FIG. 6, a and FIG. 9C). And the proliferation was further remarkable with strong CAR-target interaction as in between MVR-CAR T cells and DR^{str}-EBV-LCLs.

After target antigen recognition, a T cell secretes lytic granules, cytokines and/or chemokines to directly kill the target cell and activate immune system. T cells simultaneously exhibit all these features are regarded as polyfunctional in that the T cells could efficiently suppress pathogens and tumors. (Yuan, J., et al. (2008) Proc. Natl. Acad. Sci USA 105: 20410-20415; Ding, Z. C., et al. (2012) Blood 120: 2229-2239; Chiu, Y. L., et al. (2014) J. Clin. Invest. 124: 198-208; Franzese, O., et al. (2016) Oncoimmunology 5: e1114203). Considering the weak interaction between MVR-CAR and DR^{weak}-HLA-DR, it was unclear if a MVR-CAR T cell would be sufficient for T cell function, even if it properly proliferates after recognition of D^{weak}-HLA-DR on EBV-LCL.

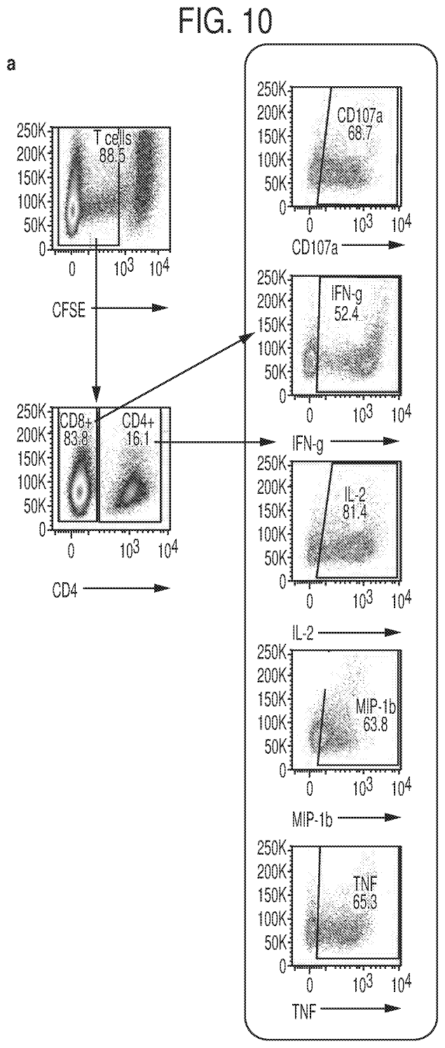

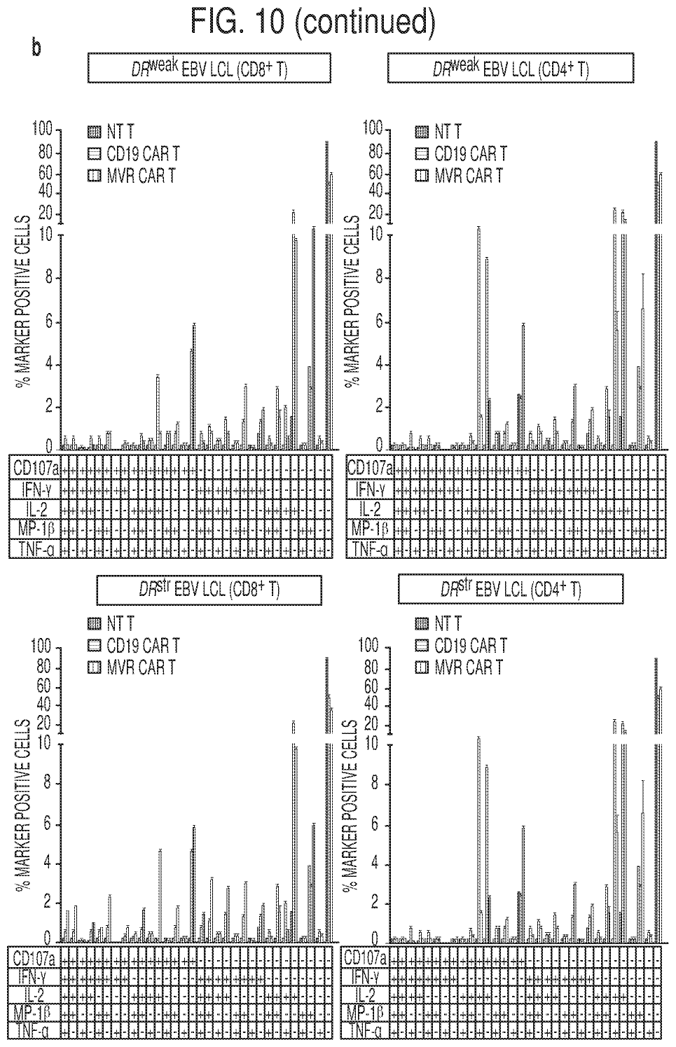

To assess polyfunctionality (i.e. simultaneous degranulation and cytokine and/or chemokine secretion), MVR-CAR T cells were assessed for simultaneous expression of five different markers, namely, IFN-γ, TNF, IL-2, MIP-1β, and CD107a, after 6 h of co-culture with EBV-LCL (FIG. 10). When co-cultured with DR^{str}-EBV-LCLs, the proportion of MVR-CAR T cells with two or more polyfunctional markers was similar to that of CD19-CAR T cells in CD4+ and CD8+ T cells (frequency of two or more markers; CD4+ MVR-CAR T=71.3%, CD4+CD19-CAR T=63.6%, CD8+ MVR-CAR T=29.4%, CD8+CD19-CAR T=24.6%; FIG. 6, b and FIG. 10). Intriguingly, co-culturing with DR^{weak}-EBV-LCLs induced polyfunctional response of MVR-CAR T cells in CD4⁺ and CD8⁺ T cells. Notably, polyfunctional capacity of CD4⁺ MVR-CAR T cells were lower than that of CD19-CAR T cells, while CD8⁺ population was not (frequency of two or more markers; CD4⁺ MVR-CAR T=31.6%, CD4⁺ CD19-CAR T=65.1%, CD8⁺ MVR-CAR T=26.3%, CD8⁺ CD19-CAR T=25.4%). In summary, these data support that D^{weak}-EBV-LCL could provide sufficient signal to cross a threshold of immune activation of MVR-CAR T cell.

An important function of CAR T cells is to induce the cell death of target cells. We assessed the cytotoxic killing efficacy of DR^{weak} MVR CAR T cells against EBV LCLs. DR^{weak} MVR CAR T cells exhibited dose-dependent killing of DR^{weak} EBV LCLs similar to the killing by CD19 CAR T cells, whereas they killed DR^{str} EBV LCLs more efficiently than CD19 CAR T cells (FIG. 6, c). Based on the limited fratricide observed during initial expansion of DR^{weak} MVR CAR T cells (FIG. 4A), these results indicate that the low affinity between DR^{weak} HLA-DR and MVR CAR can be used to distinguish EBV LCLs from activated T cells, although both express DR^{weak} HLA-DR.

CD19 CAR T cells cause on-target off-tumor toxicity such as B cell aplasia in CD19 CAR T cell-infused patients. To assess the on-target off-tumor killing efficacy of DR^{weak} MVR CAR T cells, we designed an in vitro on-target killing assay to evaluate cytotoxicity against B cells and EBV LCLs simultaneously. In agreement with their killing efficacies, CD19 CAR T and DR^{weak} MVR CAR T cells showed cytotoxic activity against DR^{str} and DR^{weak} EBV LCLs (FIG. 9B). Strikingly, DR^{weak} B cells were not affected by DR^{weak} MVR CAR T cells, whereas DR^{str} B cells were killed. To determine whether fratricidal selection and CAR downregulation affected the killing selectivity of DR^{weak} MVR CAR T cells, we subjected DR^{weak} MVR CAR T cells on day 2 and day 12 post-transduction (D2 and D12 MVR CAR T, respectively, in FIG. 7C) to an in vitro on-target killing assay. D2 MVR CAR T cells exhibited significantly higher killing activity than D12 (unpaired two-tailed t-test; LCLs, p=0.0050; B cells, p=0.0285; FIG. 9A) against both DR^{weak} B cells and DR^{weak} EBV LCLs, indicating that fratricidal selection and CAR downregulation modulated the cytotoxicity threshold. Taken together, these observations suggest that DR^{weak} MVR CAR T cells are activated by DR^{weak} EBV LCLs and exclusively kill DR^{weak} EBV LCLs; this killing is further improved by downregulation of MVR CAR. As downregulation of surface CAR occurs autonomously during fratricidal selection and eventually results in sensitivity tuning. In some instances, we refer to this process as ‘autotuning’. Thus, HLA-DR CAR T cells that are subject to fratricidal selection and CAR downregulation can specifically target and kill malignant cells.

### Example 4—Specific Targeting Depends on Antigen and CAR Levels

This example describes characterization of the property of specific targeting to malignant cells exhibited by exemplary HLA-DR CAR T cells of the present disclosure. DR^{weak} B cells were more susceptible to cell death when co-cultured with HLA-DR CAR T cells cultured for two days (D2, ‘untuned’) than with HLA-DR CAR T cells cultured for twelve days (D12, ‘autotuned’) cells (FIG. 7C and FIG. 9A). The extent of cell death, however, was still lower than that of DR^{weak} EBV LCLs. This indicates that another factor makes DR^{weak} EBV LCLs more susceptible to cytotoxicity induced by DR^{weak} MVR CAR T cells. One possible factor is the presence of death receptors, as EBV LCLs express Fas and TRAIL-R2, which induce cell death after binding to FasL and TRAIL (Xiang, Z. et al. (2014) Cancer Cell 26: 565-576). To analyze this effect, blocking agents were used to block the four major pathways of cytotoxic killing (FasL, TRAIL, perforin-1, and granzyme B)(Martinez-Lostao, L., et al., (2015) Clin. Cancer. Res. 21: 5047-5056) and the killing efficacy of CAR T cells was evaluated. Inhibition of killing by blocking agents did not differ between DR^{weak} MVR CAR T cells and CD19 CAR T cells. Blocking of FasL and TRAIL had little or no effect on killing efficacy, while inhibition of perforin-1 or granzyme B reduced killing efficacy by 15-20% (not shown). This suggests that the cell death of DR^{weak} EBV LCLs mainly involves the cytolytic granule-mediated pathway, but not death receptor-mediated pathways.

Another possible factor that makes DR^{weak} EBV LCLs more susceptible to cytotoxic killing is upregulation of HLA-DR (Zhang, Q. et al. (1994) Eur. J. Immunol. 24: 1467-1470)., as an increased level of the target antigen results in more efficient killing by CAR T cells (Caruso, H. G. et al. (2015) Cancer Res. 75: 3505-3518; Liu, X. et al. (2015) Cancer Res. 75: 3596-3607). Therefore, we investigated changes in the expression of CD19 and HLA-DR on the surface of B cells and EBV LCLs. HLA-DR was upregulated in all tested donors after transformation with EBV (B cells=42,590±2,458, EBV LCLs=78,513±8,963, mean±s.e.m., n=6), whereas CD19 was downregulated in four donors and upregulated in only two donors (FIG. 9I). To examine the contribution of DR^{weak} HLA-DR upregulation to the DR^{weak} EBV LCL-specific killing of DR^{weak} MVR CAR T cells, we assessed the susceptibility to killing of DR^{weak} HLA-DR-upregulated B cells. B cells present in lipopolysaccharide-stimulated peripheral blood mononuclear cells (PBMCs) expressed higher levels of HLA-DR than those in unstimulated PBMCs (FIG. 9D). HLA-DR expression on B cells peaked at 2-3 days post-stimulation, and the peak level was similar to that on EBV LCLs (lipopolysaccharide-stimulated B cells on day 2=86,383±7,217, day 3=82,945±6,395, mean±s.e.m., n=6). We used DR^{weak} PBMCs stimulated with lipopolysaccharide for 3 days as target cells in a killing assay, as well as autotuned and untuned MVR CAR T cells (with a 5.6-fold difference in CAR expression) as effector cells (FIG. 9E; autotuned=124,854±2,531, untuned=698,123±7,458, mean±s.e.m., n=4). Lipopolysaccharide-stimulated DR^{weak} B cells were more susceptible to DR^{weak} MVR CAR T cell-induced killing than unstimulated DR^{weak} B cells (FIG. 9E). Moreover, untuned DR^{weak} MVR CAR T cells were more efficient at killing than autotuned cells. These observations indicate that both autotuning and HLA-DR upregulation contribute to increased cytotoxic killing.

HLA-DR CAR T cells cultures for at least 8 days (e.g., 12 days) exhibited enhanced normal/malignancy selectivity of MVR-CAR T cells (FIG. 9A), attributing the selectivity to auto-tuning alone is not fully convincing. Therefore, we sought to investigate quantitative change of HLA-DR on the target cell surface. PBMCs from 6 healthy donors were used to generate EBV-LCLs, and changes of CD19 and HLA-DR surface expression during the EBV transformation were evaluated. EBV-LCLs indicated similar or even lower level of CD19 than normal B cells with two exceptions who exhibited ˜2-fold higher level (FIG. 9I). Interestingly, HLA-DR quantities were up-regulated in all six donors after EBV transformation and notably, ˜2-fold higher in DR^{weak}-EBV-LCLs than D^{weak}-Bcells. As described earlier, we assumed that in the weak affinity of MVR-CAR, the binding quantity dictates the strength of immunological synapse and consequent pore formation and granule transfer rate. To verify the hypothesis, transferred granules after CAR T cell contact with normal B cells and EBV-LCLs were measured (FIG. 9J). Interestingly, MVR-CAR did not transferred granules in normal DR^{weak}-B cells, while strong granule transfer rate was seen in DR^{str}-B cells (FIG. 9F and FIG. 9K). On the contrary, DR^{weak}-EBV-LCLs indicated increased granule transfer rate following contact with MVR-CAR T cells, and DR^{str}-EBV-LCLs exhibited 2 to 3-times higher granule transfer rate (FIG. 9F and FIG. 9L), consistently with previous killing efficacy data (FIG. 6, c and FIG. 6, d).

Strong TCR signals induce active granule transfer from T cells to target cells^{30,31}. Therefore, the extent of transfer of granules by MVR CAR may depend on the strength of the MVR CAR-HLA-DR interaction. We measured the quantity of granules transferred over time after contact between CAR T cells and B cells or EBV LCLs. Either B cells or EBV-LCLs and each violet-labeled T cells were co-incubated for indicated time at an E:T ratio of 2:1, and were followed by intracellular staining and flow cytometry analysis for quantifying transferred granules, measures as in FIG. 9J. There was no measurable granule influx into DR^{weak} B cells for 90 min after contact with DR^{weak} MVR CAR T cells, whereas granule influx into DR^{weak} EBV LCLs was easily detected and increased over time. In contrast, granule influx into DR^{str} B cells and DR^{str} EBV LCLs was rapid after contact with DR^{weak} MVR CAR T cells and was two- to four-fold greater than with CD19 CART cells (FIG. 9F).

Lytic granules transferred from T cells actively induce apoptosis of target cells³². Time-lapse imaging of caspase 3/7-activated EBV LCLs in contact with CAR T cells revealed that CD19 CAR T and DR^{weak} MVR CAR T cells progressively increased the proportion of apoptotic DR^{str} and DR^{weak} EBV LCLs (FIG. 9G and FIG. 9H). The kinetics of the interactions were similar to those of granzyme influx, suggesting that granule transfer was the main cause of DR^{weak} MVR CAR T cell-induced cytotoxicity. Collectively, these data suggest that autotuned DR^{weak} MVR CAR T cells sense the level of DR^{weak} HLA-DR and induce the death of target cells by lytic granule transfer.

### Example 5—MVR CAR T Cells Sense Enhanced HLA-DR Level In Vivo

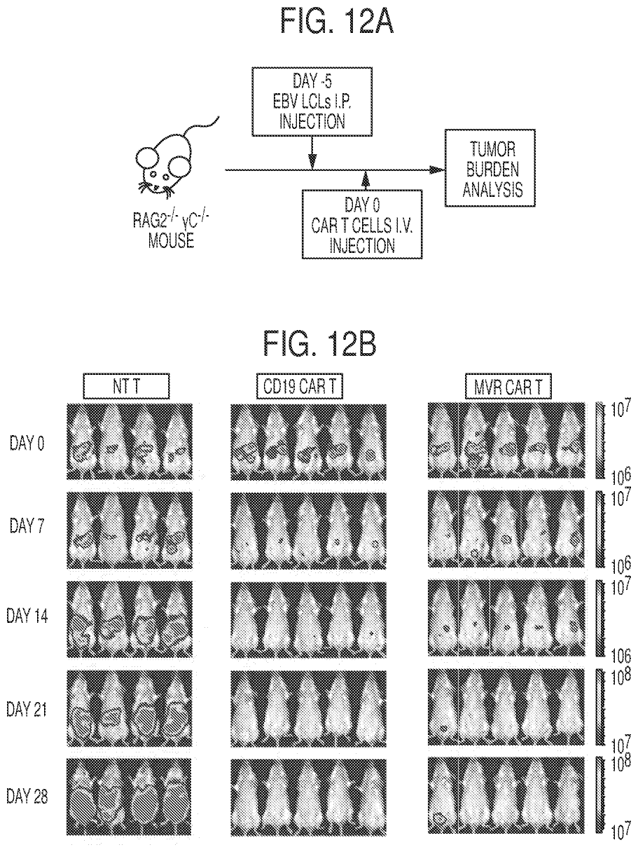

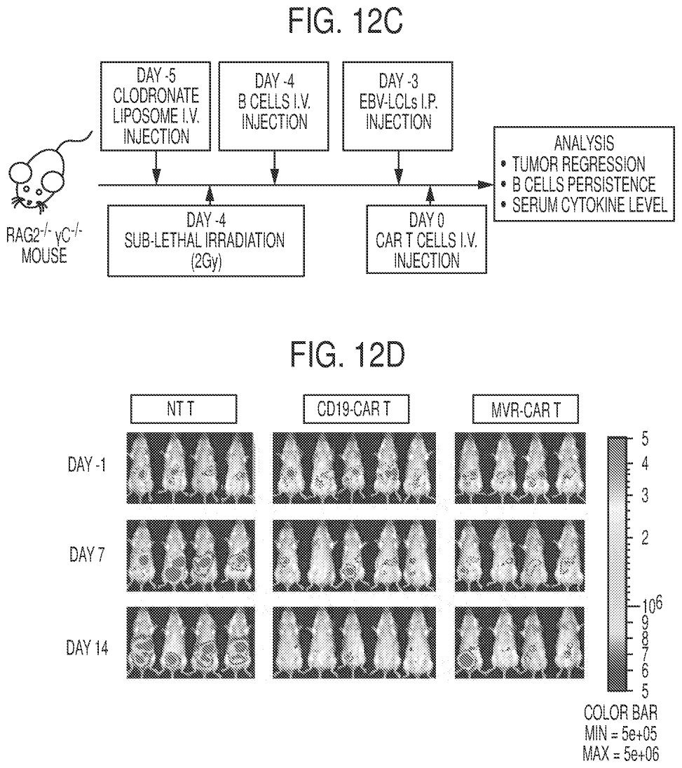

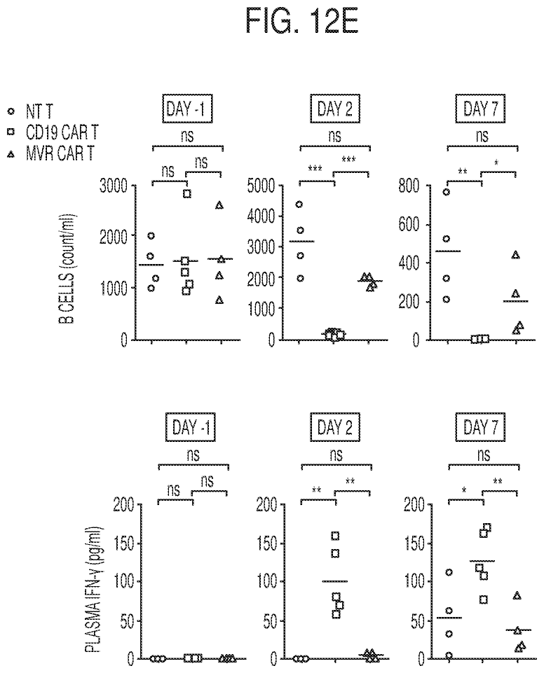

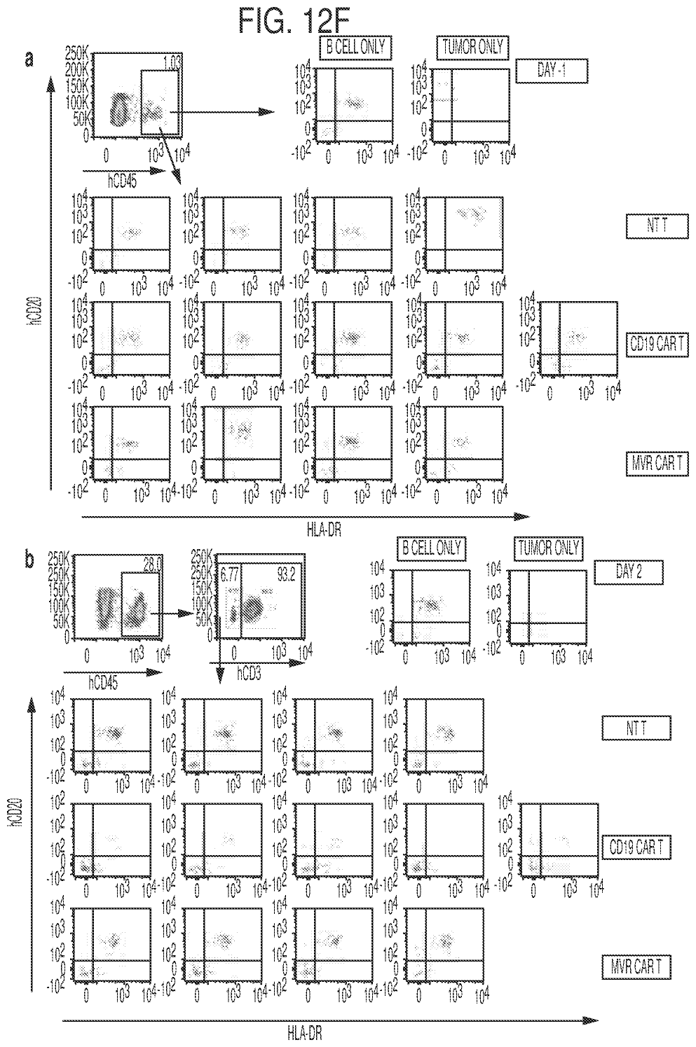

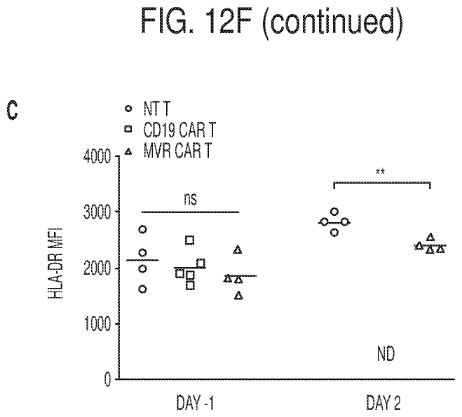

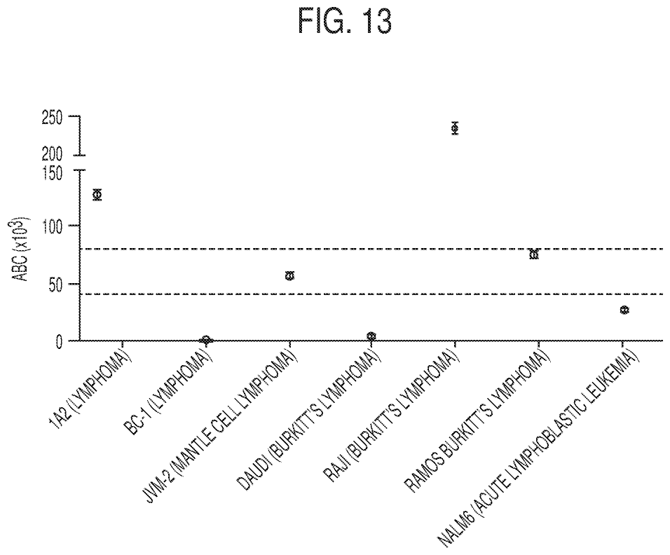

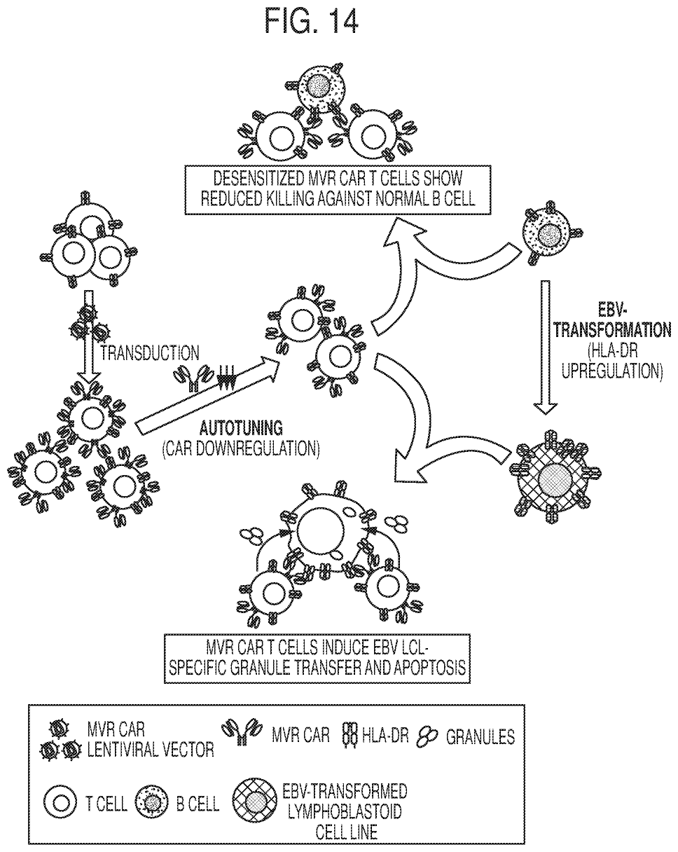

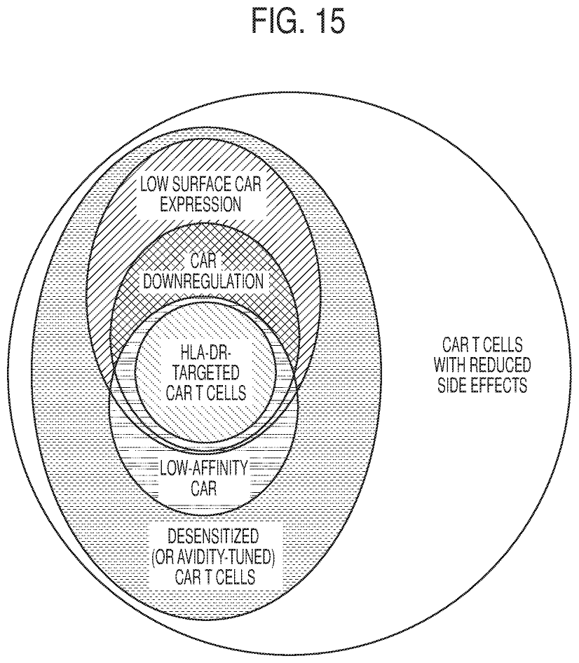

This example describes in vivo activity of exemplary HLA-DR CAR T cells of the present disclosure in an animal model. The transfer of DR^{weak} MVR CAR T cells into DR^{weak} EBV LCL-xenograft C; 129S4-Rag2^{tm1.1Flv}Il2rg^{tm1.1Flv}/J mice resulted in suppression of EBV LCL-induced tumors (FIG. 12A and FIG. 12B). The efficacy appeared higher for CD19 CAR T cells than for DR^{weak} MVR CAR T cells, although the difference was not significant (two-way ANOVA; p=0.5175). To confirm the antigen-quantity-based target-cell selectivity of DR^{weak} MVR CAR T cells under physiological conditions, we designed an in vivo on-target killing assay. In this assay, we used mice grafted with DR^{weak} B cells and DR^{weak} EBV LCLs. This enabled observation of the rate of eradication of the two cell populations in CAR T cell-infused mice (FIG. 12C). As expected, tumor regression was observed in mice infused with DR^{weak} MVR CAR T cells or CD19 CART cells, but not in those infused with NT T cells (FIG. 12D). Notably, peripheral blood DR^{weak} B cells persisted in DR^{weak} MVR CAR T cell-infused mice, whereas most DR^{weak} B cells were eliminated within two days in CD19 CART cell-infused mice (FIG. 12E and FIG. 12F, a and FIG. 12F,b). We observed a difference in the DR^{weak} B cell count between mice infused with DR^{weak} MVR CAR T cells and those infused with CD19 CAR T cells until 7 days post-T cell infusion, when tumor suppression was active. Interestingly, the expression of HLA-DR by residual DR^{weak} B cells from DR^{weak} MVR CAR T cell-infused mice was lower than by NT T cell-infused mice (FIG. 12F, c), suggesting that, as observed in vitro (FIG. 9D and FIG. 9E), HLA-DR-upregulated DR^{weak} B cells activated by xeno-reaction had increased susceptibility to DR^{weak} MVR CAR T cell-induced cytotoxicity in vivo. In addition, the plasma IFN-γ level of the DR^{weak} MVR CAR T cell-infused mice was lower than that of the CD19 CAR T cell-infused mice (FIG. 12E), in agreement with the in vitro result (FIG. 6, b and FIG. 10, b). Together, these data confirm the in vitro results showing that DR^{weak} MVR CAR T cells sense DR^{weak} HLA-DR levels under physiological conditions.

Above, the present invention has been described with reference to examples, but it can be understood by those of ordinary skill in the art that the present invention may be changed and modified in various forms without departing from the spirit and scope of the present invention, which is described in the accompanying claims.

### EQUIVALENTS

Those skilled in the art will recognize, or be able to ascertain using no more than routine experimentation, many equivalents to the specific embodiments of the invention described herein. The scope of the present invention is not intended to be limited to the above Description, but rather is as set forth in the claims.

1. A T cell comprising a chimeric antigen receptor (CAR), wherein the CAR comprises a HLA-DR antigen binding domain, wherein the T cell is autologous to a subject, and wherein the HLA-DR antigen binding domain binds to a T cell from the subject with low affinity.

2. The T cell of claim 1, wherein the HLA-DR antigen binding domain is a MVR-scFv or a variant thereof.

3. The T cell of claim 2, wherein the HLA-DR antigen binding domain comprises a heavy chain variable region with an amino acid sequence that is at least 90% identical to a sequence as set forth in SEQ ID NO: 1 and a light chain variable region with an amino acid sequence that is at least 90% identical to a sequence as set forth in SEQ ID NO: 5.

4. The T cell of claim 1, wherein the CAR further comprises an intracellular domain of the T cell receptor-ζ (TCR-ζ).

5. The T cell of claim 1, wherein the CAR further comprises a CD8α transmembrane domain and/or a 4-1BB signaling domain.

6. The T cell of claim 1, wherein the T cell has a killing efficiency for a B cell that is two time or three times lower than a killing efficiency of the T cell for an EBV LCL.

7. A pharmaceutical composition comprising: the T cell of claim 1, and a pharmaceutically acceptable carrier.

8. A method of treating cancer comprising: administering to a subject a composition that comprises or delivers the T cell of claim 1.

9. A method of producing an autologous engineered T cell, comprising:
(a) obtaining a HLA-DR antigen binding domain, wherein HLA-DR antigen binding domain binds to HLA-DR from a subject with low affinity, and
(b) expressing a chimeric antigen receptor (CAR) comprising the HLA-DR antigen binding domain in a T cell obtained from the subject, thereby producing the autologous engineered T cell.

10. (canceled)

11. The method of claim 9, wherein the HLA-DR antigen binding domain is a MVR-scFv or a variant thereof.

12. The method of claim 9, wherein the CAR further comprises an intracellular domain of the T cell receptor-ζ (TCR-ζ).

13. The method of claim 9, wherein the CAR further comprises one or both of a CD8α transmembrane domain and a 4-1BB signaling domain.

14. The method of claim 9, further comprising culturing the autologous engineered T cell in vitro for at least 8 days.

15. The method of claim 14, wherein the step of culturing produces a population of autologous engineered T cells with reduced surface expression of the CAR relative to a population of the autologous engineered T cells that has been cultured in vitro for 2 days.

16. The method of claim 14, wherein the step of culturing produces a population of autologous engineered T cells with reduced toxicity towards normal B cells relative to a population of the autologous engineered T cells that has been cultured in vitro for 2 days.

17. The method of claim 14, wherein the step of culturing produces a population of autologous engineered T cells that has enhanced selectivity for malignant cells over to non-malignant cells relative to a population of the autologous engineered T cells that has been cultured in vitro for 2 days.

18. The method of claim 14, wherein the autologous engineered T cell induce granule transfer to EBV LCLs at a level that is at least two times more than that to normal B cells from the subject.

19. A method of treating cancer comprising,
administering to the subject a composition that comprises or delivers the autologous engineered T cell prepared by the method of claim 14.

20. The method of claim 19, wherein in the cancer is a hematologic cancer.

21. The method of claim 20, wherein the hematologic cancer is selected from B-cell acute lymphoid leukemia (“BALL”), T-cell acute lymphoid leukemia (“TALL”), acute lymphoid leukemia (ALL), chronic myelogenous leukemia (CML), chronic lymphocytic leukemia (CLL), B cell prolymphocytic leukemia, blastic plasmacytoid dendritic cell neoplasm, Burkitt's lymphoma, diffuse large B cell lymphoma, follicular lymphoma, hairy cell leukemia, small cell-follicular lymphoma, large cell-follicular lymphoma, a malignant lymphoproliferative condition, MALT lymphoma, mantle cell lymphoma, Marginal zone lymphoma, multiple myeloma, myelodysplasia, non-Hodgkin's lymphoma, plasmablastic lymphoma, plasmacytoid dendritic cell neoplasm, and Waldenstrom macroglobulinemia.

22. The method of claim 19, wherein the subject has been administered or will be administered one or more additional anticancer therapies selected from ionizing radiation, a chemotherapeutic agent, an antibody agent, and a cell-based therapy, such that the subject receives treatment with both.


SEQUENCE LISTING (31 sequences)

SEQ ID NO: 1 - protein, 121 aa, Artificial
  synthetic polypeptide
QVQLKESGPGLVAPSQSLSITCTVSGFSLSRYSVHWVRQPPGKGLEWLGMIWGGGSTDYNSALKSRLSISKDNSKSQVFLKMNSLQTDDTAMYYCARNEGDTTAGTWFAYWGQGTLVTVSA

SEQ ID NO: 2 - protein, 5 aa, Artificial
  synthetic polypeptide
RYSVH

SEQ ID NO: 3 - protein, 16 aa, Artificial
  synthetic polypeptide
MIWGGGSTDYNSALKS

SEQ ID NO: 4 - protein, 17 aa, Artificial
  synthetic polypeptide
CARNEGDTTAGTWFAYW

SEQ ID NO: 5 - protein, 107 aa, Artificial
  synthetic polypeptide
DIQMTQSSSYLSVSLGGRVTITCKASDHINNWLAWYQQKPGNAPRLLISGATSLETGVPSRFSGSGSGKDYTLSITSLQTEDVATYYCQQYWSTPFTFGSGTKLEIK

SEQ ID NO: 6 - protein, 11 aa, Artificial
  synthetic polypeptide
KASDHINNWLA

SEQ ID NO: 7 - protein, 7 aa, Artificial
  synthetic polypeptide
GATSLET

SEQ ID NO: 8 - protein, 9 aa, Artificial
  synthetic polypeptide
QQYWSTPFT

SEQ ID NO: 9 - protein, 487 aa, Artificial
  synthetic polypeptide
MALPVTALLLPLALLLHAARPDIQMTQSSSYLSVSLGGRVTITCKASDHINNWLAWYQQKPGNAPRLLISGATSLETGVPSRFSGSGSGKDYTLSITSLQTEDVATYYCQQYWSTPFTFGSGTKLEIKGGGGSGGGGSGGGGSQVQLKESGPGLVAPSQSLSITSTVSGFSLSRYSVHWVRQPPGKGLEWLGMIWGGGSTDYNSALKSRLSISKDNSKSQVFLKMNSLQTDDTAMYYCARNEGDTTAGTWFAYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 10 - protein, 15 aa, Artificial
  synthetic polypeptide
GGGGSGGGGSGGGGS

SEQ ID NO: 11 - protein, 21 aa, Artificial
  synthetic polypeptide
MALPVTALLLPLALLLHAARP

SEQ ID NO: 12 - protein, 6 aa, Artificial
  synthetic polypeptide
HHHHHH

SEQ ID NO: 13 - protein, 8 aa, Artificial
  synthetic polypeptide
DYKDDDDK

SEQ ID NO: 14 - DNA, 20 bp, Artificial
  synthetic oligonucleotide
caggcagcattgaagtcagg

SEQ ID NO: 15 - DNA, 23 bp, Artificial
  synthetic oligonucleotide
gttatcaccctttactgcaaacg

SEQ ID NO: 16 - DNA, 22 bp, Artificial
  synthetic oligonucleotide
ctcctgctgaacttcactctca

SEQ ID NO: 17 - DNA, 20 bp, Artificial
  synthetic oligonucleotide
tcggagtcaacggatttggt

SEQ ID NO: 18 - DNA, 20 bp, Artificial
  synthetic oligonucleotide
ttcccgttctcagccttgac

SEQ ID NO: 19 - protein, 30 aa, Artificial
  synthetic polypeptide
FFNGTERVRFLDRYFYNQEEYVRFDSDVGE

SEQ ID NO: 20 - protein, 30 aa, Artificial; identical to SEQ ID NO: 19
  synthetic polypeptide

SEQ ID NO: 21 - protein, 29 aa, Artificial
  synthetic polypeptide
FFNGTERVRFLDRYFYNQEESVRFDSDVG

SEQ ID NO: 22 - protein, 29 aa, Artificial
  synthetic polypeptide
FFNGTERVRFLDRYFYNQEESVRFDSDVG

SEQ ID NO: 23 - protein, 30 aa, Artificial
  synthetic polypeptide
FFNGTERVRFLDRYFYHQEEYVRFDSDVGE

SEQ ID NO: 24 - protein, 30 aa, Artificial
  synthetic polypeptide
FFNGTERVRFLDRYFHNQEENVRFDSDVGE

SEQ ID NO: 25 - protein, 30 aa, Artificial
  synthetic polypeptide
FFNGTERVRYLDRYFHNQEENVRFDSDVGE

SEQ ID NO: 26 - protein, 30 aa, Artificial
  synthetic polypeptide
FFNGTERVRLLERHFHNQEELLRFDSDVGE

SEQ ID NO: 27 - protein, 30 aa, Artificial
  synthetic polypeptide
FFNGTERVQFLERLFYNQEEFVRFDSDVGE

SEQ ID NO: 28 - protein, 30 aa, Artificial
  synthetic polypeptide
FFNGTERVRFLERYFHNQEENVRFDSDVGE

SEQ ID NO: 29 - protein, 30 aa, Artificial
  synthetic polypeptide
FFNGTERVRLLERCIYNQEESVRFDSDVGE

SEQ ID NO: 30 - protein, 30 aa, Artificial
  synthetic polypeptide
FFNGTERVRYLHRGIYNQEENVRFDSDVGE

SEQ ID NO: 31 - protein, 30 aa, Artificial
  synthetic polypeptide
FFNGTERVRLLERRVHNQEEYARYDSDVGE